(19) United States
(12) Patent Application Publication
(10) Pub. No.: US 2025/0073351 A1
(43) Pub. Date: Mar. 6, 2025

(54) IMPROVED COMPOSITIONS FOR DELIVERY OF MRNA

(71) Applicant: TRANSLATE BIO, INC., Waltham, MA (US)

(72) Inventors: Shrirang KARVE, Waltham, MA (US); Neha KAUSHAL, Waltham, MA (US); Asad KHANMOHAMMED, Waltham, MA (US); Frank DEROSA, Waltham, MA (US)

(21) Appl. No.: 18/286,029

(22) PCT Filed: Apr. 19, 2022
(86) PCT No.: PCT/US2022/025335

§ 371 Date: Oct. 6, 2023

Related U.S. Application Data
(60) Provisional application No. 63/251,372, filed on Oct. 1, 2021.
(60) Provisional application No. 63/176,549, filed on Apr. 19, 2021.

(51) Int. Cl. A61K 48/00; A61K 9/00; A61P 11/00

(52) U.S. Cl. CPC A61K 48/005; A61K 9/0078; A61K 48/0033; A61K 48/0075; A61P 11/00

(57) ABSTRACT

The present invention provides, among other things, improved mRNA-encapsulating lipid nanoparticles that are particularly effective for pulmonary delivery by nebulization. The lipid nanoparticles comprise a lipid component consisting of a cationic lipid, a non-cationic lipid, a PEG-modified lipid, and a cholesterol or cholesterol analogue with a lower molar ratio of the non-cationic lipid than is typically present in lipid nanoparticles delivered via this route of administration.

### CROSS-REFERENCE TO RELATED APPLICATIONS

This application claims priority to U.S. Provisional Patent Application No. 63/176,549, filed Apr. 19, 2021, and U.S. Provisional Patent Application No. 63/251,372, filed on Oct. 21, 2021, each of which are hereby incorporated by reference in their entireties.

### BACKGROUND

Messenger RNA therapy (MRT) is becoming an increasingly important approach for the treatment of a variety of diseases. MRT involves administration of messenger RNA (mRNA) to a patient in need of the therapy for production of the protein encoded by the mRNA within the patient's body. Lipid nanoparticles are commonly used to encapsulate mRNA for efficient in vivo delivery of mRNA

The success of mRNA therapeutics will depend on a well-designed delivery system, which should guide the mRNA into the desired compartment of the selected cells. However, humans and other organisms have developed natural barriers which protect their body against different kinds of pathogens or intruders. Delivery of mRNA therapeutics into the lungs is particularly challenging. For example, nebulization of lipid nanoparticles encapsulating mRNA (e.g., an mRNA encoding a therapeutic protein) has been found to be effective to induce expression of the mRNA-encoded protein in the far reaches of the lung. However, delivering large amounts of intact lipid nanoparticles using nebulization has proven to be challenging. Moreover, lungs contain mucus, which entraps microbes and particles removing them from the lungs via the coordinated beating of motile cilia. Therefore, a need exists to provide improved lipid nanoparticles for the delivery of mRNA by nebulization to target cells in the lungs.

Nebulization output rate and change in encapsulation efficiency are key design control criteria (DC criteria) for effective pulmonary delivery of lipid nanoparticles encapsulating mRNA. Based on previous research, the inventors have identified a nebulization output rate of at least 12 ml/h and a change in post-nebulization encapsulation efficiency (ΔEE %) of no greater than 20% as critical for effective pulmonary delivery of intact mRNA into the lung.

### SUMMARY OF THE INVENTION

The present invention provides lipid nanoparticles encapsulating mRNA that are particularly effective at delivering mRNA to the lungs via nebulization. Notably, the lipid nanoparticles described herein are capable of achieving increased nebulization output rates, maintaining the encapsulation efficiency of the mRNA upon nebulization, and resulting in increased protein expression of the mRNA-encoded protein. Without being bound by theory, these improvements can be attributed to the lower molar ratio of the non-cationic lipid that is present in the lipid nanoparticles of the invention. Surprisingly, the inventors discovered that lipid nanoparticles with reduced total lipid content were non-inferior in their in vivo potency relative to lipid nanoparticles with higher lipid content, while maintaining better encapsulation efficiency and being more efficiently nebulized. In particular, the inventors found that the nebulization properties and in vivo potency of an mRNA-encapsulating lipid nanoparticle can be improved by adjusting the total lipid:mRNA ratio (mg:mg) to 19:1 or less. This can be achieved by increasing the molar ratio of the cationic lipid to greater than 40% (molar ratio) and reducing the molar ratio of the non-cationic lipid content. Specifically, increasing the molar ratio of the cationic lipid to greater than 40% (e.g., to 50% or 60%,) while reducing the overall lipid content through a reduction of the non-cationic lipid content, results in lipid nanoparticle formulations with improved in vivo potency.

The lipid nanoparticles of the invention and compositions comprising the same can be used for effective treatment or prevention of a large number of diseases and disorders (including pulmonary diseases and disorders, e.g., protein deficiencies or neoplastic diseases affecting the lungs, as well as infectious diseases, e.g., through immunization via the lungs), or for the systemic delivery of mRNA therapeutics via the lungs.

In particular, the invention provides, among other things, a lipid nanoparticle comprising:
(i) an mRNA encapsulated within the lipid nanoparticle, and
(ii) a lipid component consisting of the following components:
   a. a cationic lipid component,
   b. a non-cationic lipid component,
   c. a PEG-modified lipid component, and
   d. cholesterol component
wherein:
(1) the cationic lipid component is greater than 40% (molar ratio);
(2) the non-cationic lipid component is less than 25% (molar ratio); and
(3) a total lipid:mRNA ratio (mg:mg) is 19:1 or less.

In some embodiments, the total lipid:mRNA ratio (mg:mg) is between 11:1 and 19:1.

In some embodiments, the cationic lipid component is 45%-60% (molar ratio). In particular embodiments, the cationic lipid component is 45%-55% (molar ratio). In a specific embodiment, the cationic lipid component is about 50% (molar ratio).

In some embodiments, the non-cationic lipid component is about 22.5% (molar ratio), or less. In some embodiments, the non-cationic lipid component is less than 18% (molar ratio). In some embodiments, the non-cationic lipid component is 15% (molar ratio), or less. In some embodiments, the non-cationic lipid component is less than 13% (molar ratio).

In some embodiments, the cholesterol component is cholesterol or a cholesterol analogue.

In particular embodiments, the molar ratios of the lipid components are:
a. about 47%-60% cationic lipid,
b. about 10%-22.5% non-cationic lipid,
c. about 3%-5% PEG-modified lipid, and
d. the remainder is cholesterol or a cholesterol analogue.

In particular embodiments, the molar ratios of the lipid components are:
a. about 50%-55% cationic lipid,
b. about 10-15% non-cationic lipid,
c. about 3-5% PEG-modified lipid, and
d. the remainder is cholesterol or cholesterol analogue.

In a specific embodiment, the molar ratios of the lipid components are:
a. about 55% cationic lipid,
b. about 10% non-cationic lipid,
c. about 5% PEG-modified lipid, and
d. about 30% cholesterol or cholesterol analogue.

In another specific embodiment, the molar ratios of the lipid components are:
a. about 50% cationic lipid,
b. about 12.5% non-cationic lipid,
c. about 5% PEG-modified lipid, and
d. about 32.5% cholesterol or cholesterol analogue.

In a further specific embodiment, the molar ratios of the lipid components are:
a. about 50% cationic lipid,
b. about 15% non-cationic lipid,
c. about 5% PEG-modified lipid, and
d. about 30% cholesterol or cholesterol analogue.

In a specific embodiment, the molar ratios of the lipid components are:
a. about 47% cationic lipid,
b. about 22.5% non-cationic lipid,
c. about 3% PEG-modified lipid, and
d. about 27.5% cholesterol or cholesterol analogue.

In a particular embodiment, the cationic lipid is SY-3-E14-DMAPr. In another particular embodiment, the cationic lipid is TL1-01D-DMA.

For example, in specific embodiment, the lipid nanoparticle is any one of the lipid nanoparticles in Tables A. B, C, D, E, F, G and H.

In some embodiments, the total lipid:mRNA ratio (mg:mg) is about 18:1 or less. In some embodiments, the total lipid:mRNA ratio (mg:mg) is about 17:1 or less. In some embodiments, the total lipid:mRNA ratio (mg:mg) is about 15:1 or less.

Typically, a lipid nanoparticle in accordance with the invention is capable of being nebulized at a nebulization output rate of greater than about 12 ml/h. In particular embodiments, the lipid nanoparticle is capable of being nebulized at a nebulization output rate of greater than about 15 ml/h. In certain embodiments, the lipid nanoparticle is capable of being nebulized at a nebulization output rate of greater than about 20 m/h.

In some embodiments, the encapsulation efficiency of the lipid nanoparticle after nebulization is no more than about 10% lower than the encapsulation efficiency of the lipid nanoparticle before nebulization. In some embodiments, the lipid nanoparticle has an encapsulation efficiency before and after nebulization of at least about 90%.

The present invention also provides a lipid nanoparticle comprising
(i) an mRNA encapsulated within the lipid nanoparticle, and
(ii) a lipid component consisting of the following lipids with molar ratios of:
   a) 41%-70% of a cationic lipid,
   b) 9%-18% of a non-cationic lipid,
   c) 2%-6% of a PEG-modified lipid, and
   d) 9%-48% of cholesterol or a cholesterol analogue.

Such a lipid nanoparticle is capable of being nebulized, e.g., at a nebulization output rate of greater than 12 ml/h.

In some embodiments, the molar ratio of the cationic lipid in a lipid nanoparticle in accordance with the invention is 45%-70%. In some embodiments, the molar ratio of the cationic lipid is 45%-65%. In some embodiments, the molar ratio of the cationic lipid is 50%-70%. In some embodiments, the molar ratio of the cationic lipid is 50%-65%. In particular embodiments, the molar ratio of the cationic lipid is 50%-60%. In one specific embodiment, the molar ratio of the cationic lipid is about 50%. In another specific embodiment, the molar ratio of the cationic lipid is about 55%. In yet a further specific embodiment, the molar ratio of the cationic lipid is about 60%.

In some embodiments, the molar ratio of the non-cationic lipid in a lipid nanoparticle in accordance with the invention is 9%-15%. In particular embodiments, the molar ratio of the non-cationic lipid is 10%-15%. In a specific embodiment, the molar ratio of the non-cationic lipid is about 15%. In another specific embodiment, the molar ratio of the non-cationic lipid is about 12.5%. In yet a further specific embodiment, the molar ratio of the non-cationic lipid is about 10%.

In some embodiments, the molar ratio of the PEG-modified lipid in a lipid nanoparticle in accordance with the invention is 3%-6%. In particular embodiments, the molar ratio of the PEG-modified lipid is 4%-6%. In a specific embodiment, the molar ratio of the PEG-modified lipid is about 5%. In another specific embodiment, the molar ratio of the PEG-modified lipid is about 3%.

In some embodiments, the molar ratio of the cholesterol or cholesterol analogue in a lipid nanoparticle in accordance with the invention is 10%-45%. In particular embodiments, the molar ratio of the cholesterol or cholesterol analogue is 10%-30%. In one particular embodiment, the molar ratio of the cholesterol or cholesterol analogue is 25%-30%. In a specific embodiment, the molar ratio of the cholesterol or cholesterol analogue is about 25%. In another specific embodiment, the molar ratio of the cholesterol or cholesterol analogue is about 30%.

In one specific embodiment, the molar ratios of the lipids in a lipid nanoparticle in accordance with the invention are: a. 50%-60% cationic lipid, b. 9%-18% non-cationic lipid, c. 4%-6% PEG-modified lipid, and d. 20-35% cholesterol or cholesterol analogue. In another specific embodiment, the molar ratios of the lipids in a lipid nanoparticle in accordance with the invention are: a. 50%-60% cationic lipid, b. 9%-15% non-cationic lipid, c. 4%-6% PEG-modified lipid, and d. 25-30% cholesterol or cholesterol analogue.

In exemplary embodiments, the molar ratios of the lipids in a lipid nanoparticle in accordance with the invention are:
1) a. about 50% cationic lipid, b. about 15% non-cationic lipid, c. about 5% PEG-modified lipid, and d. about 30% cholesterol or cholesterol analogue.
2) a. about 60% cationic lipid, b. about 10% non-cationic lipid, c. about 5% PEG-modified lipid, and d. about 25% cholesterol or cholesterol analogue.
3) a. about 50% cationic lipid, b. about 10% non-cationic lipid, c. about 5% PEG-modified lipid, and d. about 35% cholesterol or cholesterol analogue.
4) a. about 50% cationic lipid, b. about 12.5% non-cationic lipid, c. about 5% PEG-modified lipid, and d. about 32.5% cholesterol or cholesterol analogue.
5) a. about 50% cationic lipid, b. about 17.5% non-cationic lipid, c. about 5% PEG-modified lipid, and d. about 27.5% cholesterol or cholesterol analogue.
6) a. about 55% cationic lipid, b. about 10% non-cationic lipid, c. about 5% PEG-modified lipid, and d. about 30% cholesterol or cholesterol analogue.
7) a. about 55% cationic lipid, b. about 12.5% non-cationic lipid, c. about 5% PEG-modified lipid, and d. about 27.5% cholesterol or cholesterol analogue. In some embodiments, the molar ratios of the lipids are: a. about 55% cationic lipid, b. about 15% non-cationic lipid, c. about 5% PEG-modified lipid, and d. about 25% cholesterol or cholesterol analogue.
8) Ia. about 55% cationic lipid, b. about 17.5% non-cationic lipid, c. about 5% PEG-modified lipid, and d. about 22.5% cholesterol or cholesterol analogue.
9) a. about 60% cationic lipid, b. about 12.5% non-cationic lipid, c. about 5% PEG-modified lipid, and d. about 22.5% cholesterol or cholesterol analogue.
10) a. about 60% cationic lipid, b. about 15% non-cationic lipid, c. about 5% PEG-modified lipid, and d. about 20% cholesterol or cholesterol analogue.

In some embodiments, a lipid nanoparticle of the present invention is any one of the lipid nanoparticles in Tables A, B, C, D, E, F, G or H.

In certain embodiments, the lipid nanoparticle of the present invention is capable of being nebulized at a nebulization output rate of greater than about 15 ml/h.

In some embodiments, the lipid nanoparticle of the present invention has an encapsulation efficiency before and after nebulization of at least about 90%. In some embodiments, the lipid nanoparticle of the present invention has an encapsulation efficiency before and after nebulization of at least about 95%. In some embodiments, the lipid nanoparticle of the present invention has an encapsulation efficiency before and after nebulization of at least about 96%. In some embodiments, the lipid nanoparticle of the present invention has an encapsulation efficiency before and after nebulization of at least about 97%. In some embodiments, the lipid nanoparticle of the present invention has an encapsulation efficiency before and after nebulization of at least about 98%. In some embodiments, the lipid nanoparticle of the present invention has an encapsulation efficiency before and after nebulization of at least about 99%.

In some embodiments, the encapsulation efficiency of the lipid nanoparticle of the present invention changes less than about 20% upon nebulization. In some embodiments, the encapsulation efficiency of the lipid nanoparticle of the present invention changes less than about 15% upon nebulization. In some embodiments, the encapsulation efficiency of the lipid nanoparticle of the present invention changes less than about 10% upon nebulization.

In some embodiments, the encapsulation efficiency of the lipid nanoparticle of the present invention after nebulization is no more than about 20% lower than the encapsulation efficiency of the lipid nanoparticle before nebulization. In some embodiments, the encapsulation efficiency of the lipid nanoparticle of the present invention after nebulization is no more than about 15% lower than the encapsulation efficiency of the lipid nanoparticle before nebulization. In some embodiments, the encapsulation efficiency of the lipid nanoparticle of the present invention after nebulization is no more than about 10% lower than the encapsulation efficiency of the lipid nanoparticle before nebulization. In specific embodiments, the encapsulation efficiency of the lipid nanoparticle after nebulization is no more than about 5% lower than the encapsulation efficiency of the lipid nanoparticle before nebulization. In certain embodiments, the encapsulation efficiency of the lipid nanoparticle after nebulization is no more than about 3% lower than the encapsulation efficiency of the lipid nanoparticle before nebulization. In some embodiments, the encapsulation efficiency of the lipid nanoparticle of the present invention after nebulization is about the same as the encapsulation efficiency of the lipid nanoparticle before nebulization.

In some embodiments, the lipid nanoparticle of the present invention is for pulmonary delivery by nebulization. In some embodiments, the nebulization is performed with a nebulizer comprising vibrating mesh technology (VMT).

In some embodiments, the cationic lipid in a lipid nanoparticle in accordance with the invention has a structure according to Formula (IIA):

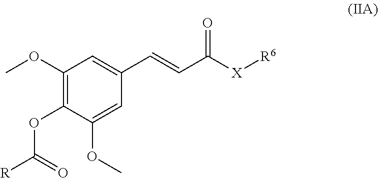

wherein X is O or S;
wherein R′ is

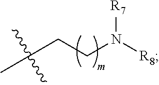

wherein R⁶ is

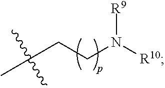

wherein m and p are each independently 0, 1, 2, 3, 4 or 5;
wherein R⁷ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₖR^{A} or —(CH₂)ₖCH(OR¹¹)R^{A};
wherein R⁸ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₙR^{B} or —(CH₂)ₙCH(OR¹²)R^{B};
wherein R⁹ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)_{q}R^{C} or —(CH₂)_{q}CH(OR¹³)R^{C};
wherein R¹⁰ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ᵣR^{D} or —(CH₂)ᵣCH(OR¹⁴)R^{D};
wherein k, n, q and r are each independently 1, 2, 3, 4, or 5;
or wherein (i) R⁷ and R⁸ or (ii) R⁹ and R¹⁰ together form an optionally substituted 5- or 6-membered heterocycloalkyl or heteroaryl wherein the heterocycloalkyl or heteroaryl comprises 1 to 3 heteroatoms selected from N, O and S;
wherein R¹¹, R¹², R¹³ and R¹⁴ are each independently selected from H, methyl, ethyl or propyl
wherein R^{A}, R^{B}, R^{C} and R^{D} are each independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)alkyl, optionally substituted —OC(O)alkenyl, optionally substituted (C₁-C₆) monoalkylamino, optionally substituted (C₁-C₆) dialkylamino, optionally substituted (C₁-C₆)alkoxy, —OH, —NH₂;
wherein at least one of R⁷, R⁸, R⁹, R¹⁰ comprises a R^{A}, R^{B}, R^{C} or R^{D} moiety respectively wherein that R^{A}, R^{B}, R^{C} or R^{D} is independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)(C₆-C₂w)alkyl or optionally substituted —OC(O)(C₆-C₂₀)alkenyl;
or a pharmaceutically acceptable salt thereof.

In some embodiments, the cationic lipid in a lipid nanoparticle in accordance with the invention has a structure according to Formula (IIID):

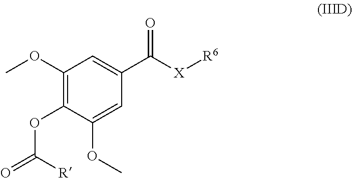

wherein X is O or S;
wherein R′ is

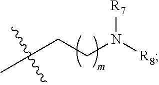

wherein R⁶ is

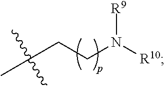

wherein m and p are each independently 0, 1, 2, 3, 4 or 5;
wherein R⁷ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₖR^{A} or —(CH₂)ₖCH(OR¹¹)R^{A};
wherein R⁸ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₙR⁸ or —(CH₂)ₙCH(OR¹²)R^{B};
wherein R⁹ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)_{q}R^{C} or —(CH₂)_{q}CH(OR¹¹)R^{C};
wherein R¹⁰ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ᵣR^{B} or —(CH₂)ᵣCH(OR¹⁴)R^{D};
wherein k, n, q and r are each independently 1, 2, 3, 4, or 5;
or wherein (i) R⁷ and R⁸ or (ii) R⁹ and R¹⁰ together form an optionally substituted 5- or 6-membered heterocycloalkyl or heteroaryl wherein the heterocycloalkyl or heteroaryl comprises 1 to 3 heteroatoms selected from N, O and S;
wherein R¹¹, R¹², R¹³ and R¹⁴ are each independently selected from H, methyl, ethyl or propyl
wherein R^{A}, R^{B}, R^{C} and R^{D} are each independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)alkyl, optionally substituted —OC(O)alkenyl, optionally substituted (C₁-C₆) monoalkylamino, optionally substituted (C₁-C₆) dialkylamino, optionally substituted (C₁-C₆)alkoxy, —OH, —NH₂;
wherein at least one of R⁷, R⁸, R⁹, R¹⁰ comprises a R^{A}, R^{B}, R^{C} or R^{D} moiety respectively wherein that R^{A}, R^{B}, R^{C} or R^{D} is independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)(C₆-C₂₀)alkyl or optionally substituted —OC(O)(C₆-C₂₀)alkenyl:
or a pharmaceutically acceptable salt thereof.

In some embodiments of Formula (IIA) or Formula (IIID), X is O.

In some embodiments of Formula (IIA) or Formula (IIID), m is 1, 2 or 3.

In some embodiments of Formula (IIA) or Formula (IIID), p is 1, 2 or 3.

In some embodiments of Formula (IIA) or Formula (IIID), R′ is:

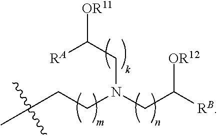

In some embodiments of Formula (IIA) or Formula (IIID), i) k, m and n=1; or ii) k, m and n=1 and R¹¹ and R¹²═H; or iii) k and n=1, and m=2 or iv) k and n=1, m=2 and R¹¹ and R¹²═H; or v) k and n=1, and m=3; or vi) k and n=1, m=3 and R¹¹ and R¹²═H.

In some embodiments of Formula (IIA) or Formula (IIID), R⁶ is

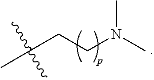

In some embodiments of Formula (IIA) or Formula (IIID), R⁶ is

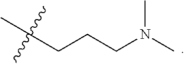 [SMILES: *CCCN(C)C]

In some embodiments of Formula (IIA) or Formula (IIID), R⁶ is selected from the group consisting of:

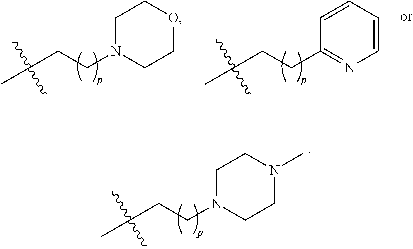

In some embodiments of Formula (IIA) or Formula (IIID). R⁶ is selected from the group consisting of:

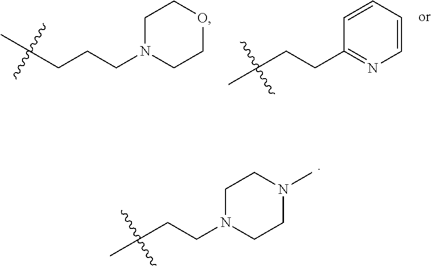

In some embodiments of Formula (IIA) or Formula (IIID), R′ is

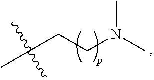

### R′ is

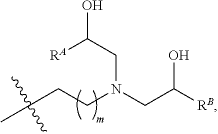

m is 2 and p is 2.

In some embodiments of Formula (IIA) or Formula (IIID), R⁶ is

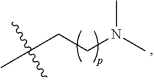

### R′ is

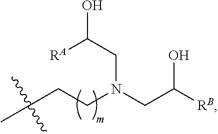

m is 3 and p is 2.

In some embodiments of Formula (IIA) or Formula (IIID), R^{A} and R^{B} are each independently selected from optionally substituted (C₆-C₂₀)alkyl, optionally substituted (C₆-C₂₀)alkenyl, optionally substituted (C₆-C₂₀)alkynyl. In some embodiments of Formula (IIA) or Formula (IIID), R^{A} and R^{B} are the same and selected from optionally substituted (C₆-C₂₀)alkyl, optionally substituted (C₆-C₂M)alkenyl, optionally substituted (C₆-C₂₀)alkynyl. In some embodiments of Formula (IIA) or Formula (IIID), R^{A} and R^{B} are each independently optionally substituted (C₆-C₂₀)alkyl. In some embodiments of Formula (IIA) or Formula (IIID), R^{A} and R^{B} are the same and are optionally substituted (C₆-C₂₀)alkyl. In some embodiments of Formula (IIA) or Formula (IIID), R^{A} and R^{B} are each independently optionally substituted (C₆-C₂₀)alkenyl. In some embodiments of Formula (IIA) or Formula (IIID), R^{A} and R^{B} are the same and are optionally substituted (C₆-C₂₀)alkenyl. In some embodiments of Formula (IIA) or Formula (IIID), R^{A} and R^{B} are each independently optionally substituted (C₆-C₂₀)alkynyl. In some embodiments of Formula (IIA) or Formula (IIID), R^{A} and R^{B} are the same and are optionally substituted (C₆-C₂₀)alkynyl. In some embodiments of Formula (IIA) or Formula (IIID), R^{A} and R^{B} are each independently optionally substituted (C₆-C₂₀)acyl. In some embodiments of Formula (IIA) or Formula (IIID), R^{A} and R^{B} are the same and are optionally substituted (C₆-C₂₀)acyl. In some embodiments of Formula (IIA) or Formula (IIID), R^{A} and R^{B} are each independently optionally substituted —OC(O)(C₆-C₂₀)alkyl. In some embodiments of Formula (IIA) or Formula (IIID), R^{A} and R^{B} are the same and are optionally substituted —OC(O)(C₆-C₂₀)alkyl. In some embodiments of Formula (IIA) or Formula (IIID), R^{A} and R^{B} are each independently optionally substituted —OC(O)(C₆-C₂₀)alkenyl. In some embodiments of Formula (IIA) or Formula (IIID), R^{A} and R^{B} are the same and are optionally substituted —OC(O)(C₆-C₂₀)alkenyl.

In some embodiments, the cationic lipid has a structure according to Formula (IIIE):

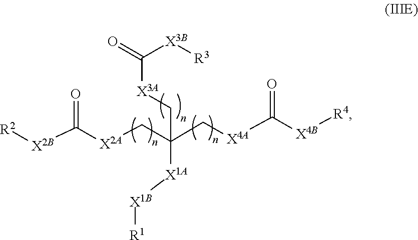

or a pharmaceutically acceptable salt thereof, wherein
each n is independently 0 or 1;
X^{1A} is independently O or NR^{1A};
R^{1A} is H or C₁-C₆ alkyl;
X^{1B} is a covalent bond, C(O), CH₂CO₂, or CH₂C(O);
one of X^{2A} and X^{2B} is O and the other is a covalent bond;
one of X^{3A} and X^{3B} is O and the other is a covalent bond;
one of X^{4A} and X^{4B} is O and the other is a covalent bond;
R¹ is independently L¹-B¹, C₆-C₃₀ alkyl, C₆-C₃₀ alkenyl, or C₆-C₃₀ alkynyl;
R² is independently L²-B², C₆-C₃₀ alkyl, C₆-C₃₀ alkenyl, C₆-C₃₀ alkynyl;
R³ is independently L³-B³, C₆-C₃₀ alkyl, C₆-C₃₀ alkenyl, C₆-C₃₀ alkynyl;
R⁴ is independently L⁴-B⁴, C₆-C₃₀ alkyl, C₆-C₃₀ alkenyl, C₆-C₃₀ alkynyl;
L¹, L², L³, and L⁴ are each independently C₁-C₃₀ alkylene; C₂-C₃₀ alkenylene; or C₂-C₃₀ alkynylene;
each of B¹, B², B³, and B⁴ is independently an ionizable nitrogen-containing group, and
wherein the cationic lipid comprises at least one ionizable nitrogen-containing group.

In some embodiments, the cationic lipids has a structure according to Formula (IIIF):

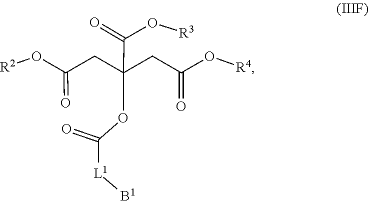

or a pharmaceutically acceptable salt thereof, wherein
B¹ is an ionizable nitrogen-containing group;
L¹ is C₁-C₁₀ alkylene;
each of R², R³, and R⁴ is independently C₆-C₃₀ alkyl, C₆-C₃₀ alkenyl, C₆-C₃₀ alkynyl.

In some embodiments, the cationic lipid has a structure according to Formula (IIIG):

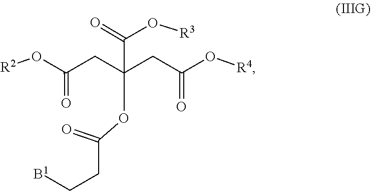

or a pharmaceutically acceptable salt thereof, wherein
B¹ is an ionizable nitrogen-containing group;
each of R², R³, and R⁴ is independently C₆-C₃₀ alkyl, C₆-C₃₀ alkenyl, C₆-C₃₀ alkynyl.

In some embodiments, each of R², R³, and R⁴ in the cationic lipid according to any of Formulae IIIE-IIIG is independently C₆-C₁₂ alkyl substituted by —O(CO)R⁵ or —C(O)OR⁵, wherein R⁵ is unsubstituted C₆-C₁₃ alkyl. In some embodiments, each of R², R³, and R⁴ in the cationic lipid according to any of Formulae IIIE-IIIG is independently:

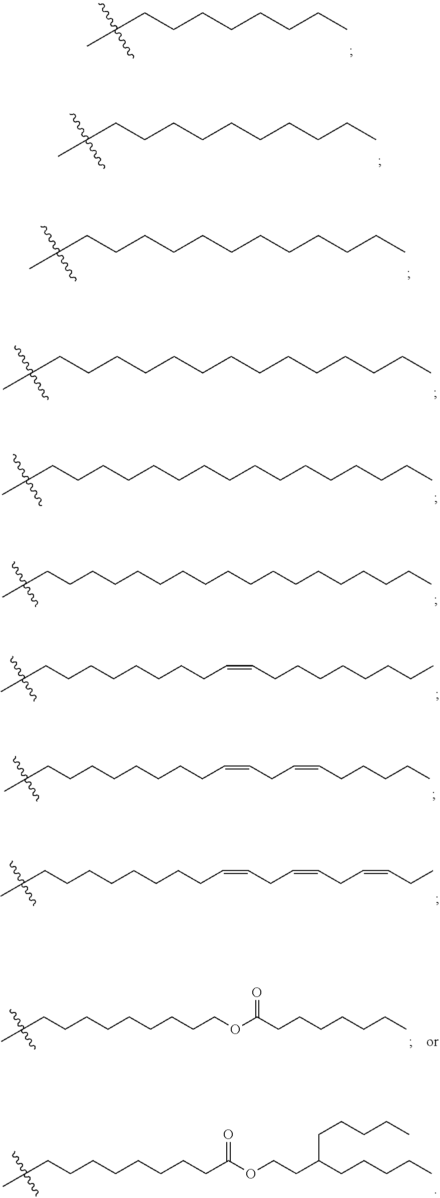

In some embodiments, B¹ in the cationic lipid according to any of Formulae IIIE-IIIG is
a) NH₂, guanidine, amidine, a mono- or dialkylamine, 5- to 6-membered nitrogen-containing heterocycloalkyl, or 5- to 6-membered nitrogen-containing heteroaryl;
b)

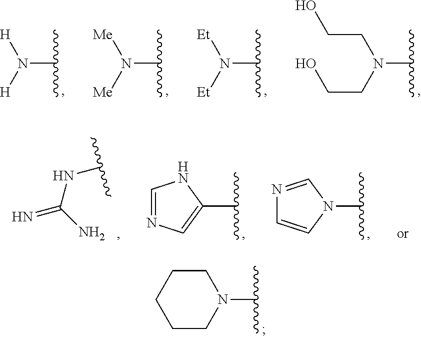

or
c)

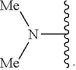 [SMILES: *N(C)C]

In some embodiments, L¹ in the cationic lipid according to any of Formulae IIIE-IIIG is C₁-alkylene.

In some embodiments, the cationic lipid in a lipid nanoparticle in accordance with the invention is selected from GL-TES-SA-DMP-E18-2, GL-TES-SA-DME-E18-2, TL1-01D-DMA, TL1-04D-DMA, SY-3-E14-DMAPr, TL1-10D-DMA, HEP-E3-E10, HEP-E4-E10, SI-4-E14-DMAPr, TL1-12D-DMA, SY-010, and SY-011. In some embodiments, the cationic lipid is SY-3-E14-DMAPr.

In some embodiments, the cationic lipid in a lipid nanoparticle in accordance with the invention is any of the cationic lipids disclosed in PCT/US21/25128, which is incorporated herein by reference.

In some embodiments, the non-cationic lipid in a lipid nanoparticle in accordance with the invention is selected from DOPE, DLoPE, DMPE, DLPE, DOPC, DEPE, DSPC, DPPC, DMPC, DOPC, DOPS, 16:1PC, and 14:1PC. In some embodiments, the non-cationic lipid is DLoPE, DMPE, DLPE or DOPC. In particular embodiments, the non-cationic lipid is DOPE. In some embodiments, the non-cationic lipid is DPPC or DSPC.

In some embodiments, the cholesterol or cholesterol analogue in a lipid nanoparticle in accordance with the invention is cholesterol. In some embodiments, the cholesterol analogue in a lipid nanoparticle in accordance with the invention is selected from β-sitosterol, stigmastanol, campesterol, fucosterol, stigmasterol, and dexamethasone. In some embodiments, the cholesterol analogue in a lipid nanoparticle in accordance with the invention is β-sitosterol. In some embodiments, the cholesterol analogue in a lipid nanoparticle in accordance with the invention is stigmastanol.

In some embodiments, the PEG-modified lipid in a lipid nanoparticle in accordance with the invention is selected from DMG-PEG2K, 2[(polyethylene glycol)-2000]-N,N-ditetradecylacetamide, and DSPE-PEG2K-COOH. In a specific embodiment, the PEG-modified lipid is DMG-PEG2K.

Exemplary lipid nanoparticles of the invention have a lipid component consisting of the following lipids:
1) a. a cationic lipid; b. DOPE as the non-cationic lipid, c. DMG-PEG2K as the PEG-modified lipid, and d. cholesterol as the cholesterol or cholesterol analogue; or
2) a. a cationic lipid, b. DSPC as the non-cationic lipid, c. DMG-PEG2K as the PEG-modified lipid, and d. cholesterol as the cholesterol or cholesterol analogue.

Exemplary lipid nanoparticles of the invention have a lipid component consisting of the following lipids:
1) a. SY-3-E14-DMAPr as the cationic lipid is, b. DOPE as the non-cationic lipid, c. DMG-PEG2K as the PEG-modified lipid, and d. cholesterol as the cholesterol or cholesterol analogue.
2) a. SY-3-E14-DMAPr as the cationic lipid, b. DSPC as the non-cationic lipid, c. DMG-PEG2K as the PEG-modified lipid, and d. cholesterol as the cholesterol or cholesterol analogue.

In some embodiments, the lipid nanoparticle of the present invention is prepared using a total lipid:mRNA ratio of less than 20:1 (mg:mg). In some embodiments, the lipid nanoparticle of the present invention is prepared using a total lipid:mRNA ratio of 16-19:1 (mg:mg). In some embodiments, the lipid nanoparticle of the present invention is prepared using a total lipid:mRNA ratio of about 19:1 (mg:mg). In some embodiments, the lipid nanoparticle of the present invention is prepared using a total lipid:mRNA ratio of about 16:1 (mg:mg).

In some embodiments, the mRNA encodes a therapeutic protein.

In some embodiments, the mRNA encapsulated in a lipid nanoparticle of the invention encodes for cystic fibrosis transmembrane conductance regulator, ATP-binding cassette sub-family A member 3 protein, dynein axonemal intermediate chain 1 (DNAI1) protein, dynein axonemal heavy chain 5 (DNAH5) protein, alpha-1-antitrypsin protein, forkhead box P3 (FOXP3) protein, or one or more surfactant protein.

In some embodiments, the mRNA encapsulated in a lipid nanoparticle of the invention is codon-optimized.

In some embodiments, the mRNA encapsulated in a lipid nanoparticle of the invention comprises at least one nonstandard nucleobase. In some embodiments, the nonstandard nucleobase is a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine (e.g., N-1-methyl-pseudouridine), 2-thiouridine, and 2-thiocytidine.

In some embodiments, the mRNA encapsulated in a lipid nanoparticle of the invention encodes for cystic fibrosis transmembrane conductance regulator (CFTR). In some embodiments, the mRNA encodes for ATP-binding cassette sub-family A member 3 protein. In some embodiments, the mRNA encodes for dynein axonemal intermediate chain 1 (DNAI1) protein. In some embodiments, the mRNA encodes for dynein axonemal heavy chain 5 (DNAH5) protein. In some embodiments, the mRNA encodes for alpha-1-antitrypsin protein, forkhead box P3 (FOXP3) protein. In some embodiments, the mRNA encodes for one or more surfactant protein. In some embodiments, the mRNA encodes for surfactant A protein, surfactant B protein, surfactant C protein, or surfactant D protein.

In some embodiments, the lipid nanoparticle of the present invention has a size less than about 150 nm. In specific embodiments, the lipid nanoparticle of the present invention has a size less than about 100 nm. In particular embodiments, the lipid nanoparticle of the present invention has a size of 60-150 nm, e.g., 60-125 nm, or 60-100 nm.

In some embodiments, the lipid nanoparticle has a size of less than about 200 nm. In some embodiments, the lipid nanoparticle has a size of less than about 150 nm. In some embodiments, the lipid nanoparticle has a size of less than about 120 nm. In some embodiments, the lipid nanoparticle has a size of less than about 110 nm. In some embodiments, the lipid nanoparticle has a size of less than about 100 nm. In some embodiments, the lipid nanoparticle has a size of less than about 80 nm. In some embodiments, the lipid nanoparticle has a size of less than about 60 nm.

In some embodiments, the invention provides a composition comprising a lipid nanoparticle of the invention. In a typical embodiment, such a composition is formulated for pulmonary delivery by nebulization. In a particular embodiment, nebulization is performed with a nebulizer comprising vibrating mesh technology (VMT).

In some embodiments, a composition comprising a lipid nanoparticle of the invention further comprises one or more excipients. In some embodiments, the one or more excipients is selected from a buffer, a salt, is a sugar, or combinations thereof. In specific embodiments, the composition of the present invention further comprises a buffer. In certain embodiments, the composition of the present invention further comprises a salt. In a specific embodiment, the salt is sodium chloride. In some embodiments, the composition of the present invention further comprises a sugar. In particular embodiments, the sugar is a disaccharide. In specific embodiments, the disaccharide is sucrose or trehalose. In some embodiments, the disaccharide is at a concentration of about 4% w/v, about 6% w/v, about 8% w/v, or about 10% w/v. In some embodiments, a composition of the present invention comprises TPGS at a concentration of about 0.1% w/v to about 1% w/v, e.g., in addition to other excipients such as a disaccharide.

In some embodiments, the mRNA in a composition of the present invention is at a concentration of 0.4 to 0.8 mg/ml. In a specific embodiment, the mRNA is at a concentration of about 0.6 mg/ml.

In certain embodiments, a composition in accordance with the present invention comprises:
a. an mRNA at a concentration of about 0.6 mg/ml encapsulated in a lipid nanoparticle of the invention,
b. trehalose at a concentration of about 8% w/v, and
c. TPGS at a concentration of about 0.5% w/v.

In some embodiments, a composition comprising a lipid nanoparticle of the present invention comprises:
a. an mRNA at a concentration of about 0.6 mg/ml encapsulated in the lipid nanoparticle, and
b. sucrose at a concentration of about 8% w/v.

In certain embodiments, a composition in accordance with the present invention comprises:
a. an mRNA encapsulated in a lipid nanoparticle of the invention,
b. a disaccharide such as trehalose or sucrose at a concentration of about 3-10% w/v,
c. a buffer, optionally a phosphate buffer, and
d. a salt, optionally sodium chloride.

In some embodiments, a composition of the present invention comprises:
a. the mRNA is at a concentration of 0.4 to 0.8 mg/ml,
b. the trehalose or sucrose is at a concentration of about 4% to 6% w/v,
c. the buffer is a phosphate buffer at a concentration of 1 mM to 10 mM (pH 5-5.5), and
d. the salt is sodium chloride at a concentration of at least 75 mM.

In some embodiments, the sodium chloride is at a concentration of about 75 mM to about 200 mM.

In some embodiments, a composition of the present invention comprises:
a. the mRNA is at a concentration of about 0.4 mg/ml,
b. the disaccharide is sucrose at a concentration of about 4% w/v,
c. the buffer is a phosphate buffer at a concentration of about 2.5 mM (pH 5.5), and
d. the salt is sodium chloride at a concentration of about 150 mM.

In some embodiments, a composition of the present invention comprises:
a. the mRNA is at a concentration of about 0.4 mg/ml,
b. the disaccharide is trehalose at a concentration of about 4% w/v,
c. the buffer is a phosphate buffer at a concentration of about 10 mM (pH 5), and
d. the salt is sodium chloride at a concentration of about 150 mM.

The lipid nanoparticles or compositions of the present invention are typically for use in therapy. In such embodiments, the mRNA encodes a therapeutic protein and the therapy comprises administering the lipid nanoparticle or composition by nebulization. In some embodiments, the therapy comprises treating or preventing a disease or disorder in a subject. Accordingly, the present invention provides, among other things, a method for delivering mRNA which encodes a therapeutic protein in vivo comprising administering a lipid nanoparticle or composition of the present invention via pulmonary delivery to a subject, wherein the pulmonary delivery is via inhalation, and the composition is nebulized prior to inhalation. The present invention also provides a method of treating or preventing a disease or disorder in a subject, the method comprising administering a lipid nanoparticle or composition of the present invention via nebulization.

In some embodiments, the lipid nanoparticle or composition of the present invention is provided as a dry powder formulation. In some embodiments, the lipid nanoparticle or composition of the present invention for use in therapy is administered with a nebulizer comprising vibrating mesh technology (VMT). In some embodiments, the lipid nanoparticle or composition of the present invention is provided in lyophilized form and is reconstituted into an aqueous solution prior to nebulization. Accordingly, the mRNA encapsulated in the lipid nanoparticle of the invention is delivered into the lungs.

In some embodiments, the therapeutic protein encoded by the mRNA is expressed in the lungs of healthy subjects. In some embodiments, the therapeutic protein is a secreted protein. In some embodiments, the therapeutic protein is an antibody. In some embodiments, the therapeutic protein is an antigen.

In some embodiments, the disease or disorder which is treated or prevented with a lipid nanoparticle or composition of the present invention is a pulmonary disease or disorder, e.g., a chronic respiratory disease. In some embodiments, the disease or disorder which is treated or prevented with a lipid nanoparticle or composition of the present invention is a protein deficiency, e.g., a protein deficiency affecting the lungs. In some embodiments, the disease or disorder which is treated or prevented with a lipid nanoparticle or composition of the present invention is a neoplastic disease, e.g., a tumor. In some embodiments, the disease or disorder which is treated or prevented with a lipid nanoparticle or composition of the present invention is an infectious disease.

In some embodiments, the disease or disorder which is treated with a lipid nanoparticle or composition of the present invention is a protein deficiency. In these embodiments, the mRNA encodes the deficient protein. In some embodiments, the protein deficiency is cystic fibrosis. Accordingly, in some embodiments, the mRNA encodes cystic fibrosis transmembrane conductance regulator (CFTR). In some embodiments, the protein deficiency is primary ciliary dyskinesia. Accordingly, in some embodiments, the mRNA encodes dynein axonemal intermediate chain 1 (DNAI1) protein. In some embodiments, the protein deficiency is a surfactant deficiency. Accordingly, in some embodiments, the mRNA encodes a surfactant protein. For example, the mRNA may encode for surfactant A protein, surfactant B protein, surfactant C protein, or surfactant D protein.

In some embodiments, the disease or disorder which is treated with a lipid nanoparticle or composition of the present invention is a chronic respiratory disease. In some embodiments, the chronic respiratory disease is chronic obstructive pulmonary disease (COPD), asthma, pulmonary arterial hypertension or idiopathic pulmonary fibrosis. Accordingly, in some embodiments, the mRNA encodes a therapeutic protein for treating a symptom of a pulmonary disease or disorder. For example, in some embodiments, the mRNA encodes an antibody directed against a pro-inflammatory cytokine.

In some embodiments, the disease which is treated with a lipid nanoparticle or composition of the present invention is a neoplastic disease, e.g., a tumor. Accordingly, in some embodiments, the mRNA encodes an antibody targeting a protein expressed on the surface of neoplastic cells, e.g., the cells making up the tumor.

In some embodiments, the disease or disorder which is treated with a lipid nanoparticle or composition of the present invention is an infectious disease. In some embodiments, the infectious disease is caused by a virus. For example, in some embodiments, the mRNA encodes a soluble decoy receptor that binds a surface protein of the virus. In other embodiments, the mRNA encodes an antibody directed to a surface protein of the virus. In some embodiments, the infectious disease which is treated with a lipid nanoparticle or composition of the present invention is caused by a bacterium. Accordingly, in some embodiments, the mRNA encodes an antibody directed to a surface protein of the bacterium. In some embodiments, the mRNA encodes an antigen derived from a causative agent of the infectious disease.

In some embodiments, the subject which is treated with a lipid nanoparticle or composition of the present invention is human.

### BRIEF DESCRIPTION OF THE DRAWING

The drawings are for illustration purposes only, not for limitation.

FIG. 1 illustrates the effect of reducing the molar ratio of the non-cationic lipid on the nebulization output rate. Lowering the molar ratio of the non-cationic lipid from 30% (LNP 12) to 15% (LNP 3) improved the nebulization output rate from about 10 ml/h to about 15 ml/h. A threshold target is indicated by the dashed horizontal line in the figure. Lowering the molar ratio of the non-cationic lipid further from 15% (LNP 3) to 10% (LNP 9) improved the nebulization output rate from about 15 ml/h to about 30 ml/h. Thus, the claimed lipid nanoparticles achieve improved nebulization output rates.

FIG. 2 illustrates the effect of reducing the molar ratio of the non-cationic lipid on the change in encapsulation efficiency of the lipid nanoparticle after nebulization. This is significant as a reduction in encapsulation efficiency after nebulization means that less mRNA is encapsulated by the lipid nanoparticle and so less mRNA is available to be delivered to the lungs, which can result in reduced in vivo protein expression, e.g., because unencapsulated mRNA is rapidly degraded in vivo.

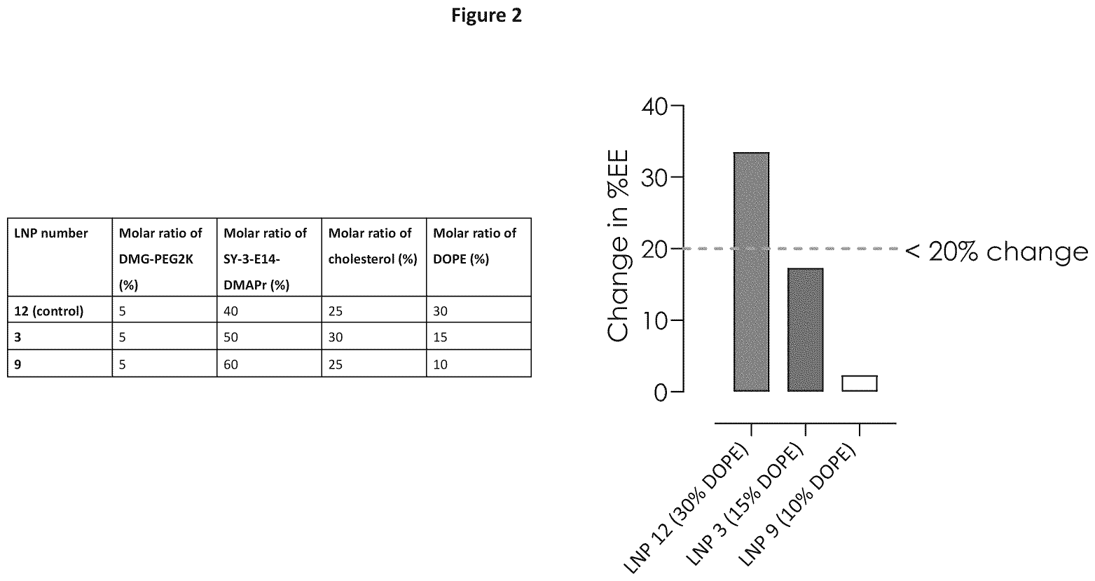

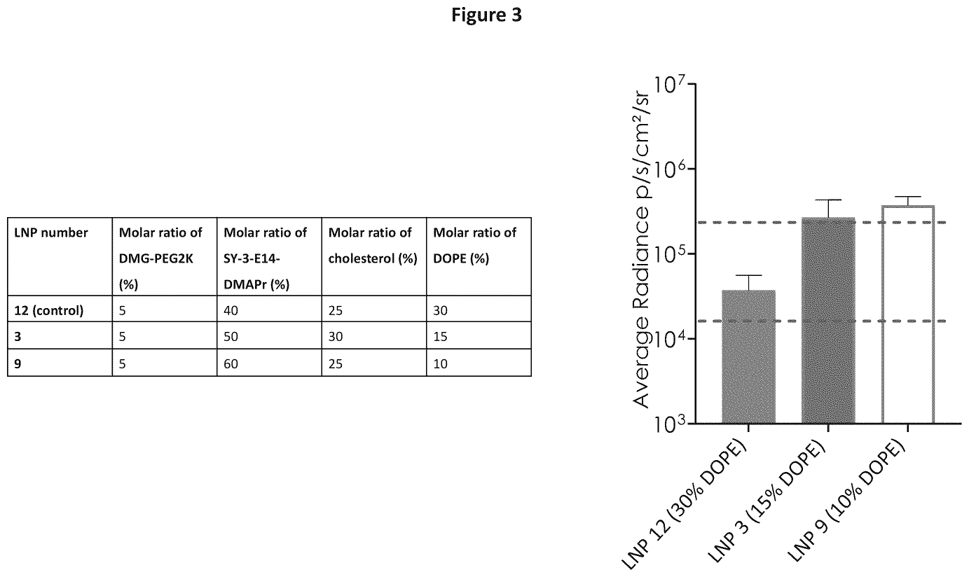

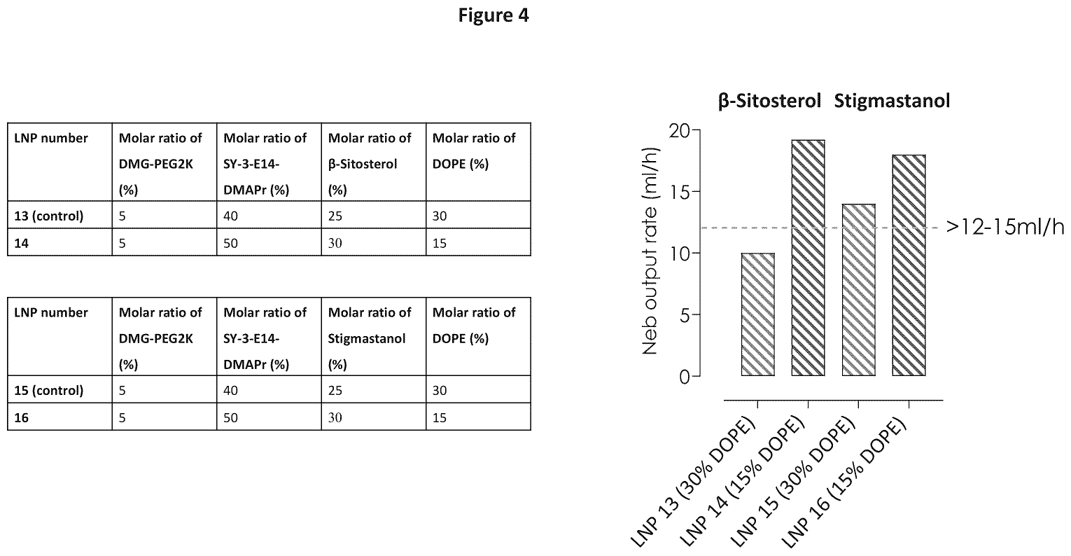

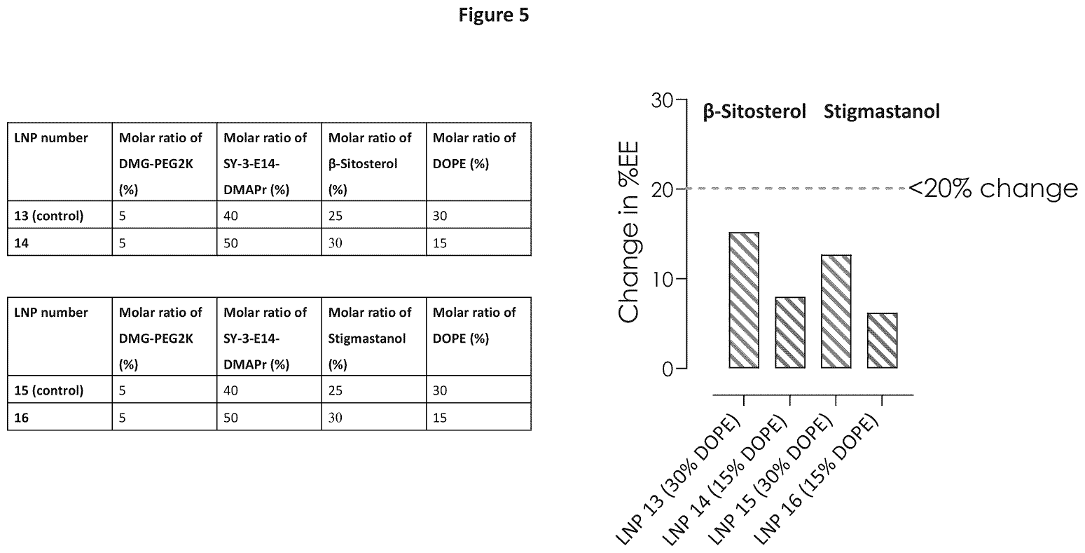

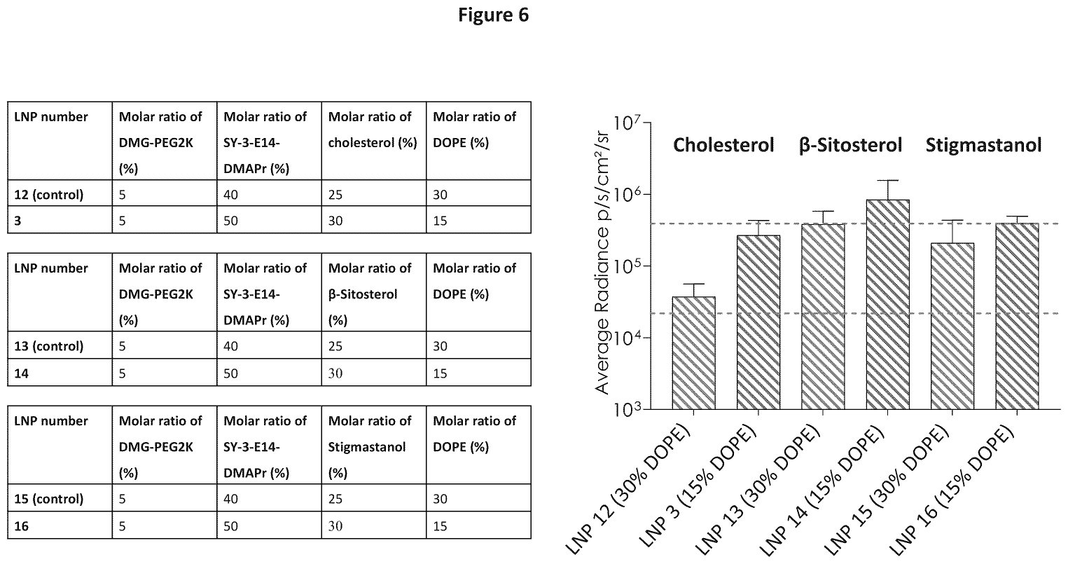

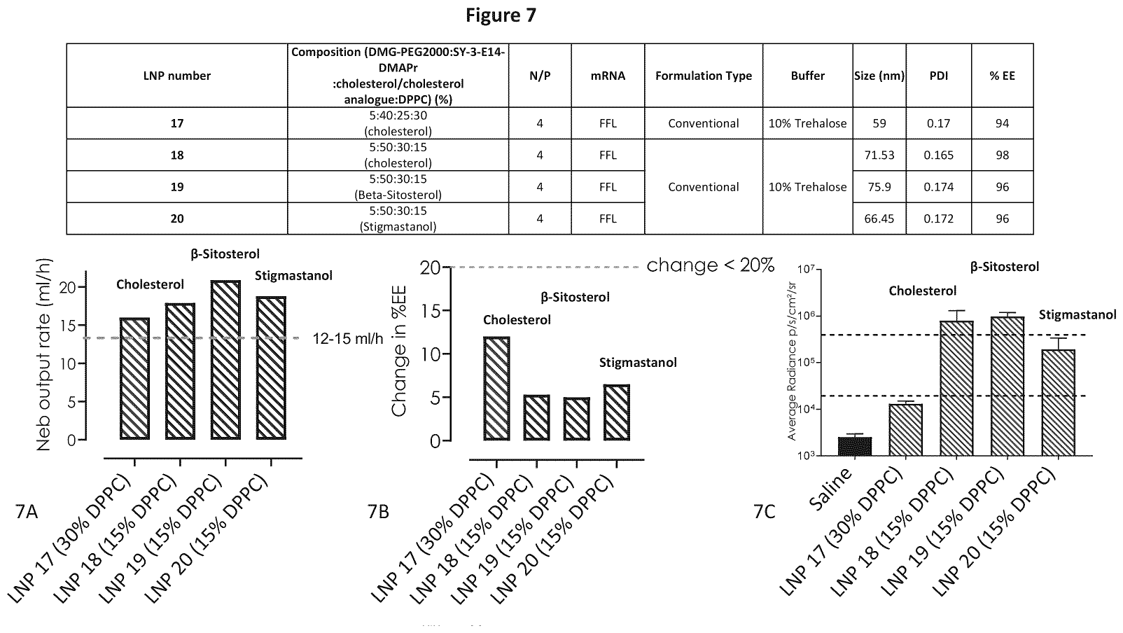

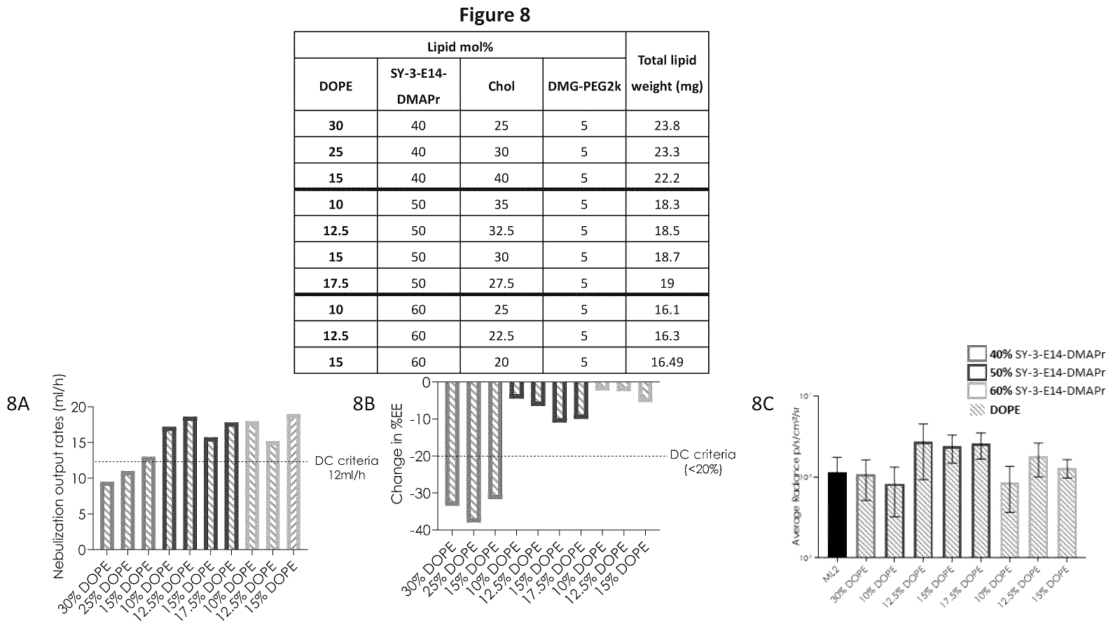

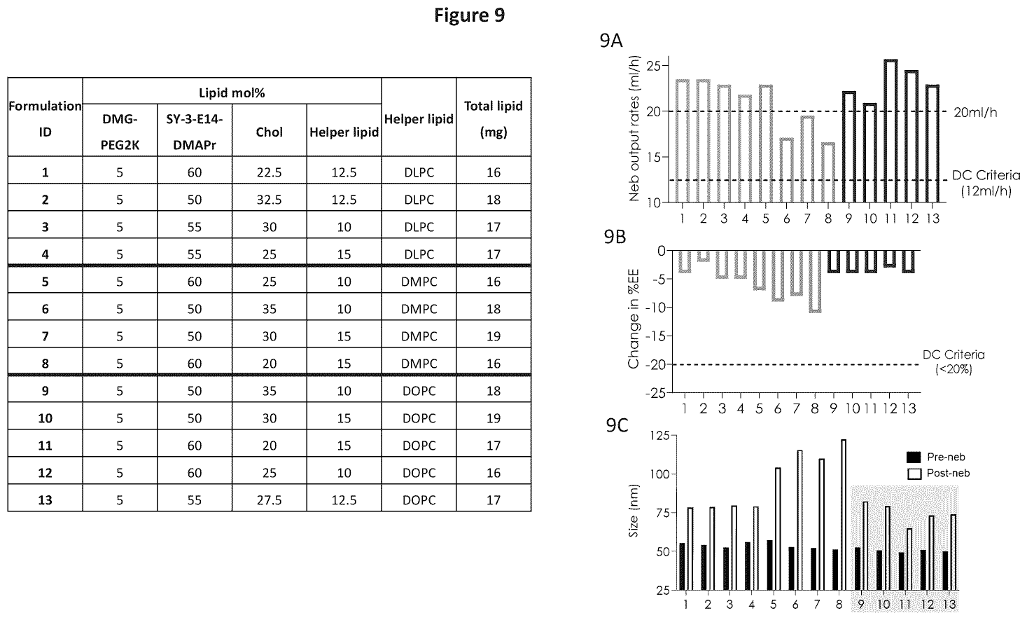

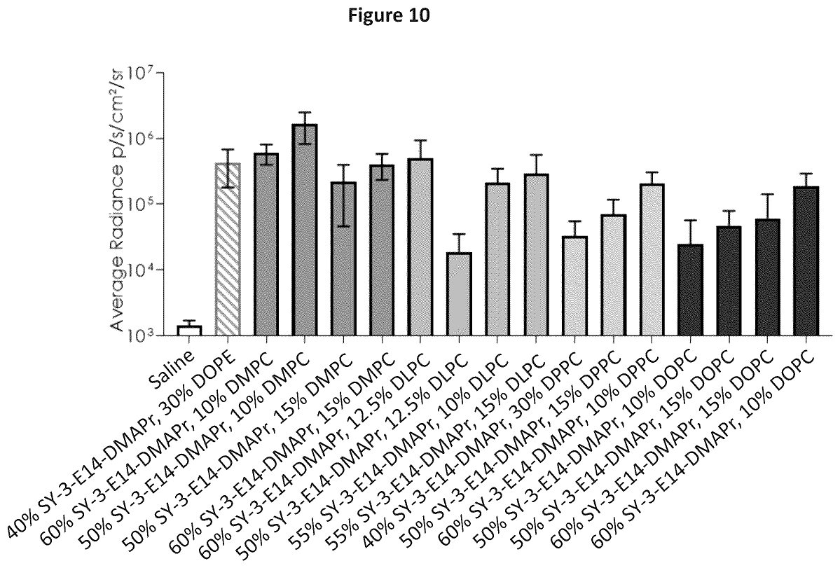

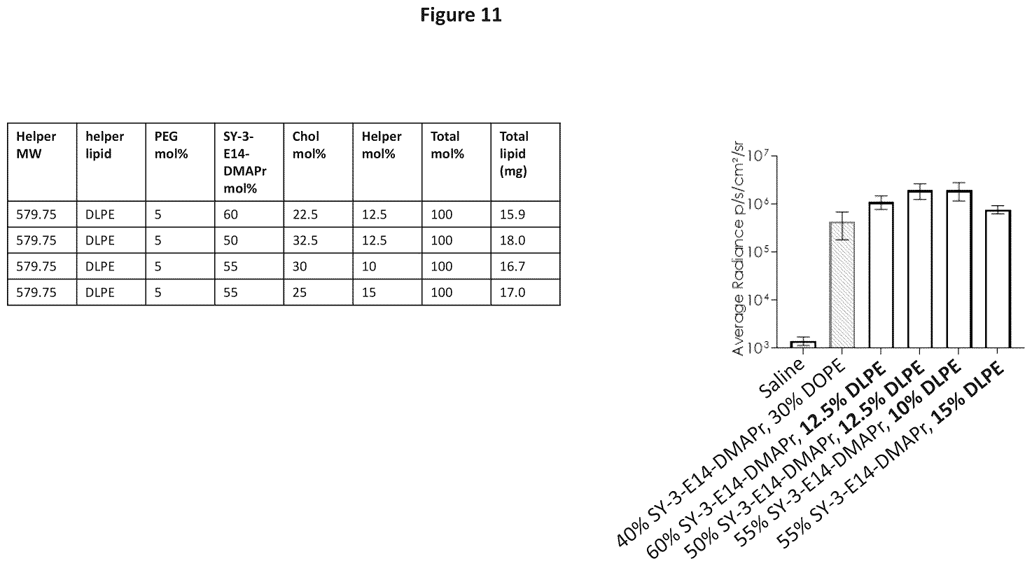

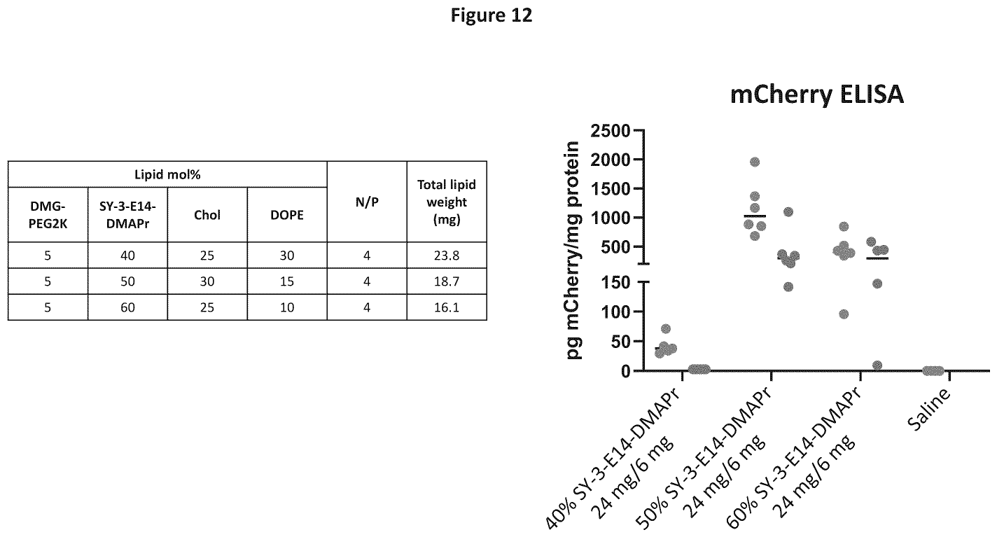

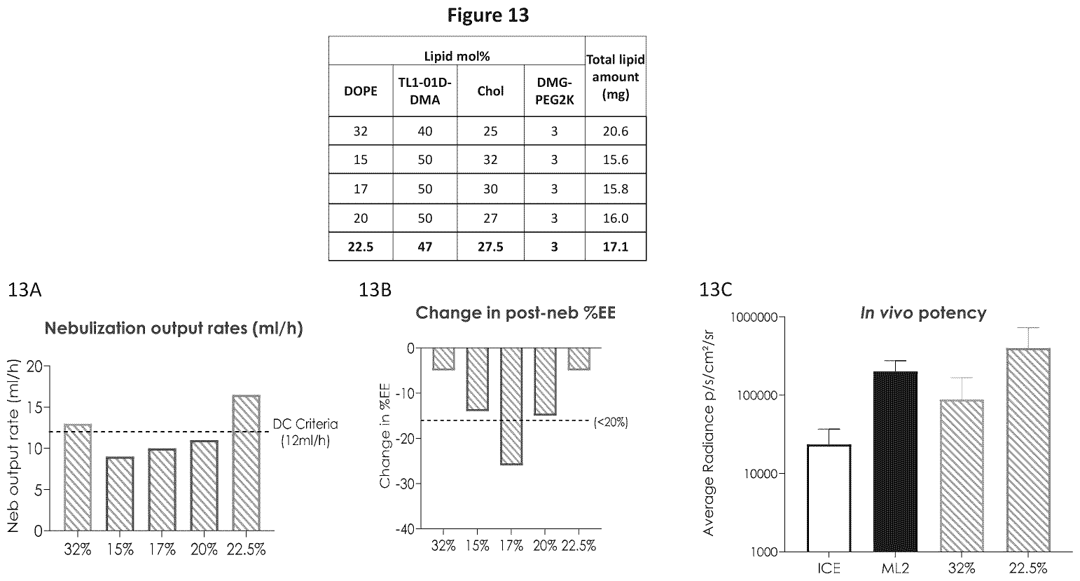

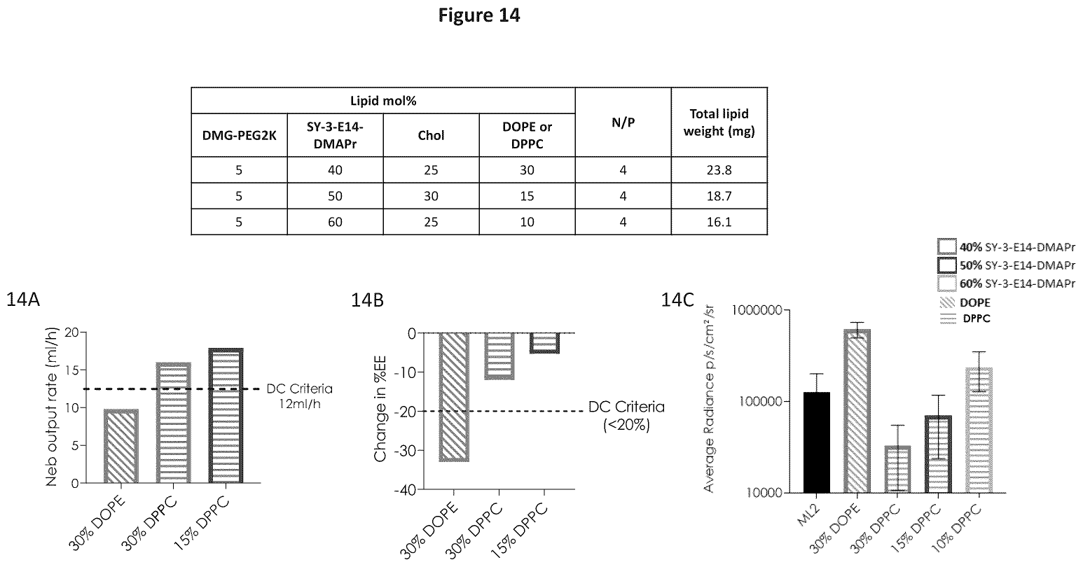

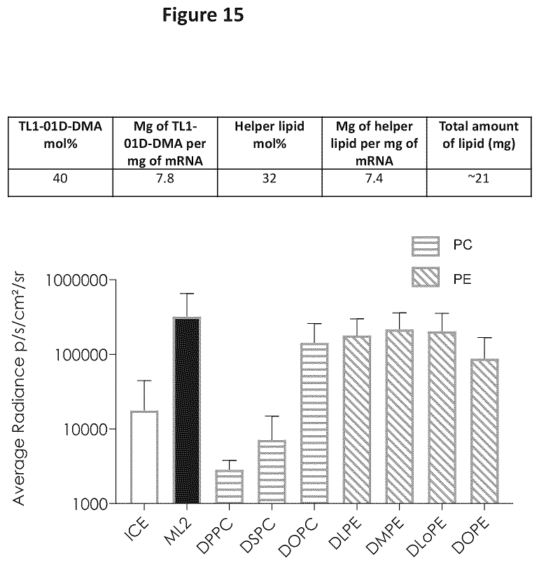

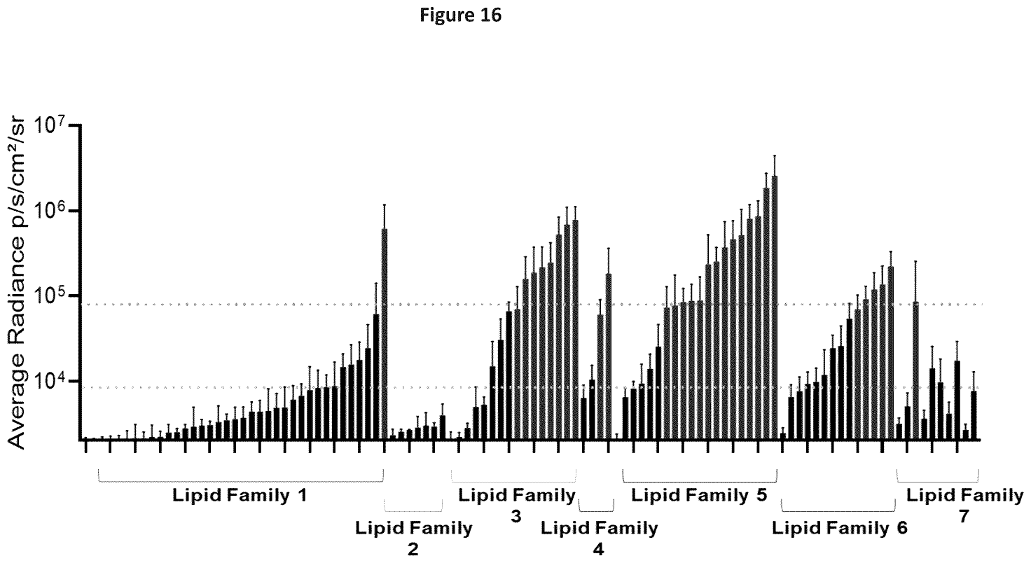

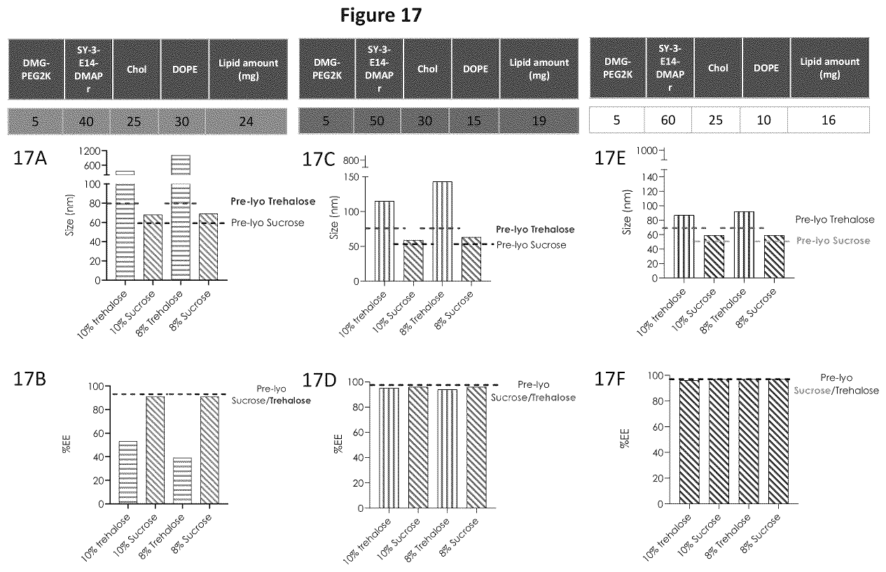

As shown in FIG. 2, lowering the molar ratio of the non-cationic lipid from 30% (LNP 12) to 15% (LNP 3) reduced the loss (expressed as a percent change) in encapsulation of mRNA within the lipid nanoparticle after nebulization (as compared to encapsulation prior to nebulization) from more than 30% loss to less than 20% loss. Lowering the molar ratio of the non-cationic lipid further from 15% (LNP 3) to 10% (LNP 9) reduced the loss in encapsulation of mRNA within the lipid nanoparticle after nebulization even more, to less than a 5% loss in encapsulation efficiency. Thus, the claimed lipid nanoparticles retain more mRNA during nebulization.

FIG. 3 illustrates the effect of reducing the molar ratio of the non-cationic lipid on protein expression. Lowering the molar ratio of the non-cationic lipid from 30% (LNP 12) to 15% (LNP 3) and 10% (LNP 9) improved the amount of mRNA that was delivered and thus expressed. Reference target expression levels are shown by dashed lines. Thus, the claimed lipid nanoparticles achieve improved delivery and expression of mRNA.

FIG. 4 illustrates the effect of reducing the molar ratio of the non-cationic lipid on the nebulization output rate with the cholesterol analogues β-sitosterol and stigmastanol. Lowering the molar ratio of the non-cationic lipid from 30% (LNP 13 and LNP 15) to 15% (LNP 14 and LNP 16) improved the nebulization output rate.

FIG. 5 illustrates the effect of reducing the molar ratio of the non-cationic lipid on the loss (expressed as a percent change) in encapsulation of mRNA within the lipid nanoparticle after nebulization (as compared to encapsulation prior to nebulization) with the cholesterol analogues β-sitosterol and stigmastanol. Lowering the molar ratio of the non-cationic lipid from 30% (LNP 13 and LNP 15) to 15% (LNP 14 and LNP 16) reduced the loss in encapsulation of mRNA within the lipid nanoparticle, or encapsulation efficiency, due to nebulization.

FIG. 6 illustrates the effect of reducing the molar ratio of the non-cationic lipid on protein expression with cholesterol and the cholesterol analogues β-sitosterol and stigmastanol. Lowering the molar ratio of the non-cationic lipid from 30% (LNP 12, LNP 13 and LNP 15) to 15% (LNP 3, LNP 14 and LNP 16) improved the amount of mRNA that was delivered and thus expressed. Reference target expression levels are shown by dashed lines.

FIG. 7A illustrates the effect of reducing the molar ratio of the non-cationic lipid DPPC on the nebulization output rate. Lowering the molar ratio of the non-cationic lipid DPPC from 30% to 15% improved the nebulization output rate.

FIG. 7B illustrates the effect of reducing the molar ratio of the non-cationic lipid DPPC on the loss in encapsulation of mRNA within the lipid nanoparticle, or encapsulation efficiency, after nebulization as compared to the encapsulation efficiency prior to nebulization. Lowering the molar ratio of the non-cationic lipid DPPC from 30% to 15% reduced the loss in encapsulation efficiency of the lipid nanoparticle after nebulization by half or more.

FIG. 7C illustrates the effect of reducing the molar ratio of the non-cationic lipid DPPC on protein expression. Lowering the molar ratio of the non-cationic lipid DPPC from 30% to 15% improved the amount of mRNA that is delivered and thus expressed. Reference target expression levels are shown by dashed lines.

FIG. 8 illustrates the effect of lowering the total lipid content of lipid nanoparticles by reducing the molar ratio of the non-cationic lipid DOPE on their nebulization output rate. The composition of each of the tested lipid nanoparticles is provided in the table above the figure panels. Reducing the molar concentration of the cationic lipid from 30% to 25% to 15%, while keeping the molar concentration of the cationic lipid constant at 40% resulted in an increase of the nebulization output rate (FIG. 8A). The increase in nebulization output rate was maintained when the cationic lipid content was increased to above 40% (molar ratio) by reducing the non-cationic lipid content to 18% or less (molar ratio), in order to arrive at a total lipid:mRNA ratio (mg:mg) of 19:1 or less, while dramatically increasing the post-nebulization encapsulation efficiency (FIG. 8B). The improvements in the nebulization characteristics were associated with an increase in in vivo potency (FIG. 8C). The line in FIG. 8A indicates the DC criterion for the nebulization output rate (>12 ml/h). The line in FIG. 8B indicated the DC criterion for post-nebulization encapsulation efficiency (a ΔEE of no greater than 20%).

FIG. 9 illustrates the nebulization characteristics for lipid nanoparticle formulations with a reduced total lipid content comprising different non-cationic lipids. The composition of each of the tested lipid nanoparticles is provided in the table above the figure panels. The non-cationic lipid was DLPC (12:0PC) in bars 1-4, DMPC (14:0PC) in bars 5-8, and DOPC (18:1PC) in bars 9-13. The nebulization output rate was greatly improved with all tested formulations (FIG. 9A). The observed change in encapsulation efficiency was typically about 10% of less (FIG. 9B). While a size increase was observed, all lipid nanoparticles maintained a size of less than 150 nm (FIG. 9C).

FIG. 10 illustrates the in vivo potency of lipid nanoparticle formulations with a reduced total lipid content comprising different non-cationic lipids. The tested lipid nanoparticle compositions comprised varying concentrations of SY-3-E14-DMAPr as the cationic lipid and varying concentrations of either DOPE, DMPC, DLPC, DPPC or DOPC as the non-cationic lipid. Expression of an mRNA encoding firefly luciferase was measured by determining the average radiance (p/s/cm²/sr) of the lungs of mice after administration of a test formulation.

FIG. 11 illustrates the in vivo potency of lipid nanoparticles with reduced total lipid content comprising DLPE in place of DOPE. The composition of each of the tested lipid nanoparticle formulations is shown in the table. Expression of an mRNA encoding firefly luciferase was measured by determining the average radiance (p/s/cm²/sr) of the lungs of mice after administration of a test formulation.

FIG. 12 illustrates the in vivo potency of lipid nanoparticle formulations with reduced total lipid content comprising different molar ratios of a cationic lipid. The composition of each of the tested lipid nanoparticle formulations is shown in the table next to the graph. After pulmonary delivery via nebulization of the lipid nanoparticles, the lungs of test animals were isolated and homogenized, and mCherry expression levels were determined by ELISA. Increasing the molar ratio of the cationic lipid to greater than 40% (e.g., to 50% or 60%,) while reducing the overall lipid content through a reduction of the non-cationic lipid content, resulted in lipid nanoparticle formulations with improved in vivo potency.

FIG. 13 illustrates the nebulization characteristics and in vivo potency of lipid nanoparticle formulations comprising different amounts of DOPE and the cationic lipid TL1-01D-DMA. The composition of each of the tested lipid nanoparticle formulations is shown in the table above the figure panels. Adjusting the molar ratios of both the cationic lipid and the non-cationic lipid resulted in the identification of a lipid nanoparticle formulation with an improved nebulization output (FIG. 13A) and with high encapsulation efficiency (FIG. 13B). This formulation had a reduced total lipid content with a total lipid:mRNA ratio (mg:mg) of less than 19:1 (marked in bold in the bottom row of the table). It also displayed an improved in vivo potency relative to comparator lipid nanoparticle compositions comprising ICE or ML-2 as the cationic lipid component and in comparison to the starting composition with a higher total lipid content (FIG. 13C).

FIG. 14 illustrates that decreasing the total lipid content can resulted in improved nebulization characteristics and in vivo potency of lipid nanoparticle formulations comprising the non-cationic lipid DPPC. The composition of each of the tested lipid nanoparticle formulations is shown in the table above the figure panels. Reducing the total lipid content by decreasing the molar ratio of DPPC to 15% while increasing the molar ratio of the cationic lipid (SY-3-E14-DMAPr) to 50% resulted in an increased nebulization output rate (FIG. 14A) and in an improved post-nebulization encapsulation efficiency (FIG. 14B). The in vivo potency also increased and could be improved further by adjusting the molar ratio of SY-3-E14-DMAPr to 60% and of DPPC to 10% (FIG. 14C). Surprisingly, none of the lipid nanoparticle formulations comprising DPPC had an in vivo potency that was comparable to a standard lipid nanoparticle formulation containing 30% DOPE. However, the formulation comprising 60% SY-3-E14-DMAPr and 10% DPPC compared favourably to an ML2-based lipid nanoparticle. In vivo potency was assessed by determining the average radiance (p/s/cm²/sr) of the lungs of mice after administration of a test formulation that induced expression of an mRNA encoding firefly luciferase.

FIG. 15 illustrates the effect of different PE lipids and PC lipids as the non-cationic lipid component of lipid nanoparticles on in vivo potency. The PC lipids DPPC, DSPC and DOPC, and the PE lipids DLPE, DMPE, DLoPE and DOPE were evaluated. The composition of the lipid nanoparticle formulations is shown in the table above the graph. With the exception of DOPC, administration of lipid nanoparticles with PE lipids as the non-cationic lipid resulted in higher mRNA expression levels than lipid nanoparticles with PC lipids as the non-cationic lipid. Expression of an mRNA encoding firefly luciferase was measured by determining the average radiance (p/s/cm²/sr) of the lungs of mice after administration of a test formulation.

FIG. 16 is an exemplary bar graph that depicts the amount of radiance produced by luciferase protein expressed in mice after administration of mRNA-lipid nanoparticles, each comprising a different cationic lipid component. The horizontal line in the graph around 10⁴ p/s/cm²sr represents the historical radiance/expression of pulmonary delivered FFL mRNA encapsulated in a lipid nanoparticle comprising a first reference cationic lipid. The horizontal line in the graph around 10⁶ p/s/cm²sr represents the historical radiance/expression of pulmonary delivered FFL mRNA encapsulated in a lipid nanoparticle comprising a second reference cationic lipid. These thresholds can be used to screen lipids for pulmonary delivery. The lower threshold is representative of the minimum expression level that is typically desired for lipid nanoparticles of the invention.

FIG. 17 illustrates the effect of different disaccharides (trehalose, sucrose) and disaccharide concentrations (10%, 8%) on the size (FIGS. 17A, 17C and 17E) and encapsulation efficiency (FIGS. 17B, 17D and 17F) of mRNA-encapsulating lipid nanoparticle formulations before and after lyophilization. The composition of each of the tested lipid nanoparticles is provided in the tables above the graphs. The dashed lines indicate the size and encapsulation efficiency of the lipid nanoparticles before lyophilization (“pre-lyo”), respectively. The bars show the size and encapsulation efficiency after lyophilization and reconstitution of the lipid nanoparticles.

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 5%, 4%, 3%, 2%, or 1% in either direction (greater than or less than; e.g., +2.5%) of the stated reference value, unless otherwise stated or otherwise evident from the context (except where such number would exceed 100% of a possible value).

Patient: As used herein, the term ‘patient’ or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Stable: As used herein, the term “stable” refers to a composition that retains its physical stability and/or biological activity. In one embodiment, stability is determined based on the percentage of mRNA which is degraded (e.g., fragmented). In another embodiment, stability is determined based on the percentage of lipid nanoparticles that are no longer in suspension.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. In some embodiments, a subject can be a patient, which refers to a human presenting to a medical provider for diagnosis or treatment of a disease or disorder. The term “subject” is used herein interchangeably with “individual” or “patient.” A subject can be afflicted with or is susceptible to a disease or disorder but may or may not display symptoms of the disease or disorder. In some embodiments, a subject may be healthy and receive a lipid nanoparticle or composition of the invention for the prevention of a disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{Z}—(C═O)—, wherein R^{Z} is, for example, any alkyl, alkenyl, alkynyl, heteroalkyl or heteroalkylene.

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls). C₁-C₂₀ alkenyls (e.g., linear or branched C₄-C₂₀ dienyls, linear or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₈-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR′, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the aliphatic is unsubstituted. In embodiments, the aliphatic does not include any heteroatoms. Alkyl: As used herein, the term “alkyl” means acyclic linear and branched hydrocarbon groups, e.g., “C₁-C₃₀ alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR′, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“C₁-C₅₀ alkyl”). In some embodiments, an alkyl group has 1 to 40 carbon atoms (“C₁-C₄₀ alkyl”). In some embodiments, an alkyl group has 1 to 30 carbon atoms (“C₁-C₃₀ alkyl”). In some embodiments, an alkyl group has 1 to 20 carbon atoms (“C₁-C₂₀ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₀ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C6). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁-C₅₀ alkyl. In certain embodiments, the alkyl group is a substituted C₁-C₅₀ alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms. Alkenyl: As used herein, “alkenyl” means any linear or branched hydrocarbon chains having one or more unsaturated carbon-carbon double bonds that may occur in any stable point along the chain, e.g. “C₂-C₃O alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“C₂-C₅₀ alkenyl”). In some embodiments, an alkenyl group has 2 to 40 carbon atoms (“C₂-C₄₀ alkenyl”). In some embodiments, an alkenyl group has 2 to 30 carbon atoms (“C₂-C₃₀ alkenyl”). In some embodiments, an alkenyl group has 2 to 20 carbon atoms (“C₂-C₂₀ alkenyl”). In some embodiments, an alkenyl group has 2 to 10 carbon atoms (“C₂-C₁₀ alkenyl”). In some embodiments, an alkenyl group has 2 to 9 carbon atoms (“C₂-C₉ alkenyl”). In some embodiments, an alkenyl group has 2 to 8 carbon atoms (“C₂-C₈ alkenyl”). In some embodiments, an alkenyl group has 2 to 7 carbon atoms (“C₂-C₇ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in I-butenyl). Examples of C₂-C₄ alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C6), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂-C₅₀ alkenyl. In certain embodiments, the alkenyl group is a substituted C₂-C₅₀ alkenyl.

Alkynyl: As used herein, “alkynyl” means any hydrocarbon chain of either linear or branched configuration, having one or more carbon-carbon triple bonds occurring in any stable point along the chain, e.g., “C₂-C₃₀ alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR, —OCOR″, —OCO₂R″, —NH₂, —NHR″, —N(R″)₂, —SR″ or —SO₂R″, wherein each instance of R″ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R″ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R″ independently is unsubstituted C₁-C₃ alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“C₂-C₅₀ alkynyl”). An alkynyl group that has one or more triple bonds and one or more double bonds is also referred to as an “ene-yne”. In some embodiments, an alkynyl group has 2 to 40 carbon atoms (“C₂-C₄₀ alkynyl”). In some embodiments, an alkynyl group has 2 to 30 carbon atoms (“C₂-C₃M alkynyl”). In some embodiments, an alkynyl group has 2 to 20 carbon atoms (“C₂-C₂₀ alkynyl”). In some embodiments, an alkynyl group has 2 to 10 carbon atoms (“C₂-C₁₀ alkynyl”). In some embodiments, an alkynyl group has 2 to 9 carbon atoms (“C₂-C₉ alkynyl”). In some embodiments, an alkynyl group has 2 to 8 carbon atoms (“C₂-C₈ alkynyl”). In some embodiments, an alkynyl group has 2 to 7 carbon atoms (“C₂-C₇ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-triple bonds can be internal (such as in 2-butynyl) or terminal (such as in 1-butynyl). Examples of C₂-C₄ alkynyl groups include, without limitation, ethynyl (C₂), 1-propynyl (C₃), 2-propynyl (C₃), 1-butynyl (C₄), 2-butynyl (C₄), and the like. Examples of C₂-C₆ alkenyl groups include the aforementioned C₂-C₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂-C₅₀ alkynyl. In certain embodiments, the alkynyl group is a substituted C₂-C₅₀ alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”, e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆-C₁₄ aryl. In certain embodiments, the aryl group is a substituted C₆-C₁₄ aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“C₃-C₁₀ carbocyclyl”) and zero heteroatoms in the non-aromatic ring system. In some embodiments, a carbocyclyl group has 3 to 8 ring carbon atoms (“C₃-C₈ carbocyclyl”). In some embodiments, a carbocyclyl group has 3 to 7 ring carbon atoms (“C₃-C₇ carbocyclyl”). In some embodiments, a carbocyclyl group has 3 to 6 ring carbon atoms (“C₃-C₆ carbocyclyl”). In some embodiments, a carbocyclyl group has 4 to 6 ring carbon atoms (“C₄-C₆ carbocyclyl”). In some embodiments, a carbocyclyl group has 5 to 6 ring carbon atoms (“C₅-C₆ carbocyclyl”). In some embodiments, a carbocyclyl group has 5 to 10 ring carbon atoms (“C₅-C₁₀ carbocyclyl”). Exemplary C₃-C₆ carbocyclyl groups include, without limitation, cyclopropyl (C₃), cyclopropenyl (C₃), cyclobutyl (C₄), cyclobutenyl (C4), cyclopentyl (C₅), cyclopentenyl (C₅), cyclohexyl (C₆), cyclohexenyl (C₆), cyclohexadienyl (C₆), and the like. Exemplary C₃-C₈ carbocyclyl groups include, without limitation, the aforementioned C₃-C₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃-C₁₀ carbocyclyl groups include, without limitation, the aforementioned C₃-C₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted C₃-C₁₀ carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆, cycloalkyl”). In some embodiments, a cycloalkyl group has 4 to 6 ring carbon atoms (“C₄-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃-C₁₀ cycloalkyl. In certain embodiments, the cycloalkyl group is a substituted C₃-C₁₀ cycloalkyl.

Halogen: As used herein, the term “halogen” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 n electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-4 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl. “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR^{cc})R^{bb}, —ScH, —ScR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb}), —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃ —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{aa})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁-C₅₀ alkyl, C₂-C₅₀ alkenyl, C₂-C₅₀ alkynyl, C₃-C₁₄ carbocyclyl, 3-14 membered heterocyclyl, C₆-C₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or =NOR^{cc};
each instance of R^{aa} is, independently, selected from C₁-C₅₀ alkyl, C₂-C₅₀ alkenyl, C₂-C₅₀ alkynyl, C₃-C₁₀ carbocyclyl, 3-14 membered heterocyclyl. C₆-C₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{aa})₂, —P(═O)(NR^{cc})₂, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{cc}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ee})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁-C₅₀ alkyl, C₂-C₅₀ alkenyl, C₂-C₅₀ alkynyl, C₃-C₁₀ carbocyclyl, 3-10 membered heterocyclyl, C₆-C₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of Re is, independently, selected from C₁-C₅₀ alkyl, C₂-C₅₀ alkenyl, C₂-C₅₀ alkynyl, C₃-C₁₀ carbocyclyl, C₆-C₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen. C₁-C₅₀ alkyl, C₂-C₅₀ alkenyl, C₂-C₅₀ alkynyl, C₃-C₁₀ carbocyclyl, 3-10 membered heterocyclyl, C₆-C₁₀ aryl and 5-10 membered heteroaryl, or two R^{ff} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁-C₅₀ alkyl, —ON(C₁-C₅₀ alkyl)₂, —N(C₁-C₅₀ alkyl)₂, —N(C₁-C₅₀ alkyl)₃+X⁻, —NH(C₁-C₅₀alkyl)₂+X⁻, —NH₂(C₁-C₅₀ alkyl)+X⁻, —NH₃+X⁻, —N(OC₁-C₅₀ alkyl)(C₁-C₅₀ alkyl), —N(OH)(C₁-C₅₀ alkyl), —NH(OH), —SH, —SC₁-C₅₀ alkyl, —SS(C₁-C₅₀ alkyl), —C(═O)(C₁-C₅₀ alkyl), —CO₂H, —CO₂(C₁-C₅₀ alkyl), —OC(═O)(C₁-C₅₀ alkyl), —OCO₂(C₁-C₅₀ alkyl), —C(═O)NH₂, —C(═O)N(C₁-C₅₀ alkyl)₂, —OC(═O)NH(C₁-C₅₀ alkyl), —NH—C(═O)(C₁-C₅₀ alkyl), —N(C₁-C₅₀ alkyl)C(═O)(C₁-C₅₀ alkyl), —NHCO₂(C₁-C₅₀ alkyl), —NHC(═O)N(C₁-C₅₀ alkyl)₂, —NHC(═O)NH(C₁-C₅₀ alkyl), —NHC(═O)NH₂, —C(═NH)O(C₁-C₅₀ alkyl), —OC(═NH)(C₁-C₅₀ alkyl), —OC(═NH)OC₁-C₅₀ alkyl, —C(═NH)N(C₁-C₅₀ alkyl)₂, —C(═NH)NH(C₁-C₅₀ alkyl), —C(═NH)NH₂, —OC(═NH)N(C₁-C₅₀alkyl)₂, —OC(NH)NH(C₁-C₅₀ alkyl), —OC(NH)NH₂, —NHC(NH)N(C₁-C₅₀ alkyl)₂, —NHC(═NH)NH₂, —NHSO₂(C₁-C₅₀ alkyl), —SO₂N(C₁-C₅₀ alkyl)₂, —SO₂NH(C₁-C₁₀ alkyl), —SO₂NH₂, —SO₂(C₁-C₅₀ alkyl), —SO₂O(C₁-C₅₀ alkyl), —OSO₂(C₁-C₆ alkyl), —SO(C₁-C₆ alkyl), —Si(C₁-C₅₀ alkyl)₃, —OSi(C₁-C₆ alkyl)₃, —C(═S)N(C₁-C₅₀ alkyl)₂, C(═S)NH(C₁-C₅₀ alkyl), C(═S)NH₂, —C(═O)S(C₁-C₆ alkyl), —C(═S)S(C₁-C₆ alkyl), —SC(═S)S(C₁-C₆ alkyl), —P(═O)₂(C₁-C₅₀ alkyl), —P(═O)(C₁-C₅₀ alkyl)₂, —OP(═O)(C₁-C₅₀ alkyl)₂, —OP(═O)(OC₁-C₅₀ alkyl)₂, C₁-C₅₀ alkyl, C₂-C₅₀ alkenyl, C₂-C₅₀ alkynyl, C₃-C₁₀ carbocyclyl, C₆-C₁₀ aryl, 3-10 membered heterocyclyl, 5-10 membered heteroaryl: or two geminal R^{gg} substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quaternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})?, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁-C₅₀ alkyl, C₂-C₅₀ alkenyl, C₂-C₅₀ alkynyl, C₃-C₁₀ carbocyclyl, 3-14 membered heterocyclyl, C₆-C₁₄ aryl, and 5-14 membered heteroaryl, or two R^{C} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluencsulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)?R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-12-(trimethylsilyl)ethoxylmethylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylidencamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4′-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacctamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### DETAILED DESCRIPTION

The present invention provides lipid nanoparticles encapsulating mRNA that are particularly effective at delivering mRNA to the lungs via nebulization. Notably, the lipid nanoparticles described herein achieve increased nebulization output rates, maintain encapsulation efficiency of the mRNA upon nebulization, and result in increased protein expression of the mRNA-encoded protein.

In particular, the invention provides, among other things, a lipid nanoparticle comprising:
(i) an mRNA encapsulated within the lipid nanoparticle, and
(ii) a lipid component consisting of the following components:
   a. a cationic lipid component,
   b. a non-cationic lipid component,
   c. a PEG-modified lipid component, and
   d. cholesterol component
wherein:
(1) the cationic lipid component is greater than 40% (molar ratio);
(2) the non-cationic lipid component is less than 25% (molar ratio); and
(3) a total lipid:mRNA ratio (mg:mg) is 19:1 or less.

For example, the present invention provides a lipid nanoparticle comprising
(i) an mRNA encapsulated within the lipid nanoparticle, and
(ii) a lipid component consisting of the following lipids with molar ratios of:
   a) 41%-70% of a cationic lipid,
   b) 9%-18% of a non-cationic lipid,
   c) 2%-6% of a PEG-modified lipid, and
   d) 9%-48% of cholesterol or a cholesterol analogue.

Lipid nanoparticles of the invention and compositions comprising the same can be used for effective treatment of a large number of pulmonary diseases or for the systemic delivery of mRNA therapeutics via the lungs.

### Lipid Nanoparticle

The inventors have discovered that mRNA encapsulating lipid nanoparticles with a lipid component consisting of a cationic lipid, a non-cationic lipid, a PEG-modified lipid and a cholesterol or cholesterol analogue are more effective for pulmonary administration by nebulization when a lower molar ratio of the non-cationic lipid is used than is typically present in lipid nanoparticles delivered via this route of administration. In particular, the inventors surprisingly found that they were able to achieve increased nebulization output rates and increased expression of the protein encoded by the mRNA encapsulated in the lipid nanoparticle, while also maintaining encapsulation efficiency of the mRNA upon nebulization, when they employed a lower molar ratio of non-cationic lipid.

These observations were independent of the particular non-cationic lipid used. Indeed, while the inventors observed improved nebulization when replacing cholesterol with various cholesterol analogues, the most effective way to increase output and protein expression, while maintaining encapsulation efficiency, was to lower the amount of non-cationic lipid present in the formulation. Advantageously, reducing the amount of non-cationic lipid made it possible to reduce the total amount of lipid required for the effective delivery and expression of the encapsulated mRNA.

### Molar Ratios

As used herein, the molar ratios of the lipids of the lipid nanoparticle sum to 100%. For example, for a lipid nanoparticle comprising a lipid component consisting of the following lipids with molar ratios of: a) 41%-70% of a cationic lipid, b) 9%-18% of a non-cationic lipid, c) 2%-6% of a PEG-modified lipid, and d) 9%-48% of cholesterol or a cholesterol analogue, if the molar ratio of the cationic lipid is 70%, the molar ratio of the non-cationic lipid may be 9% and the molar ratio of the PEG-modified lipid may be 2%, then the molar ratio of the cholesterol or a cholesterol analogue may be 19% (70%+9%+2%+19%=100%).

In some embodiments, where the molar ratio is defined as a range, e.g., 2%-6% of a PEG-modified lipid, the limits of the range are the exact values specified. For example, the lower limit 2% of the molar ratio 2%-6% for the PEG-modified lipid is 2%.

### Cationic Lipid

As used herein, the term “cationic lipid” refers to an ionizable lipid that has a net positive charge at a pH lower than at a physiological pH (e.g., about pH 5.5, about 6.0, or about 6.5).

Suitable cationic lipids for use in the lipid nanoparticles and compositions of the invention include the cationic lipids as described in International Patent Publication WO 2010/144740, which is incorporated herein by reference. In certain embodiments, the lipid nanoparticles and compositions of the present invention include a cationic lipid, (6Z,9Z,28Z,31Z)-heptatriaconta-6,9,28,31-tetraen-1⁹-yl 4-(dimethylamino) butanoate, having a compound structure of:

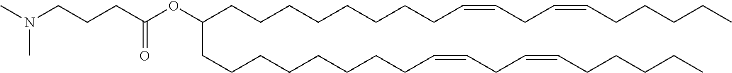 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the lipid nanoparticles and compositions of the present invention include ionizable cationic lipids as described in International Patent Publication WO 2013/149140, which is incorporated herein by reference. In some embodiments, the lipid nanoparticles and compositions of the present invention include a cationic lipid of one of the following formulas:

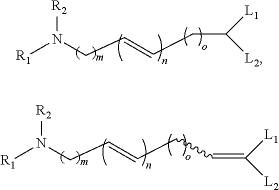

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the lipid nanoparticles and compositions of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

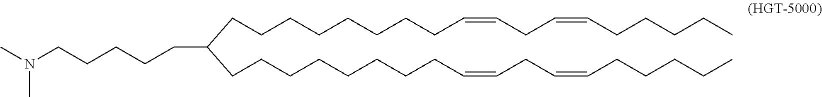 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the lipid nanoparticles and compositions of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

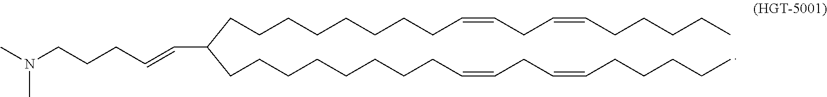 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the lipid nanoparticles and compositions of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

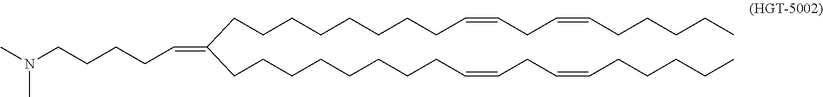 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the lipid nanoparticles and compositions of the invention include cationic lipids described as aminoalcohol lipidoids in International Patent Publication WO 2010/053572, which is incorporated herein by reference. In certain embodiments, the lipid nanoparticles and compositions of the present invention include a cationic lipid having a compound structure of:

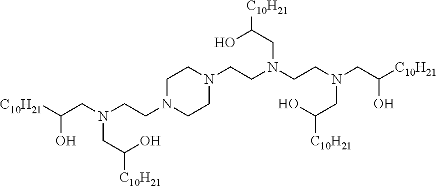

and pharmaceutically acceptable salts thereof.

Other suitable cationic lipids for use in the lipid nanoparticles and compositions of the invention include the cationic lipids as described in International Patent Publication WO 2016/118725, which is incorporated herein by reference. In certain embodiments, the lipid nanoparticles and compositions of the present invention include a cationic lipid having a compound structure of:

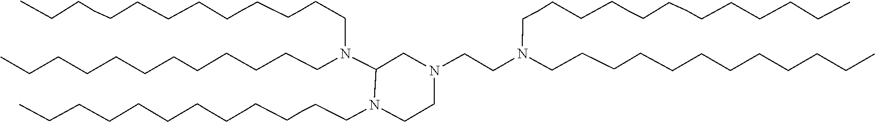 [SMILES: CCCCCCCCCCCCCCCN(CCCCCCCCCCCC)CCN1CCN(CCCCCCCCCCCCCCC)C(N(CCCCCCCCCCCC)CCCCCCCCCCCCCCC)C1]

and pharmaceutically acceptable salts thereof.

Other suitable cationic lipids for use in the lipid nanoparticles and compositions of the invention include the cationic lipids as described in International Patent Publication WO 2016/118724, which is incorporated herein by reference. In certain embodiments, the lipid nanoparticles and compositions of the present invention include a cationic lipid having a compound structure of:

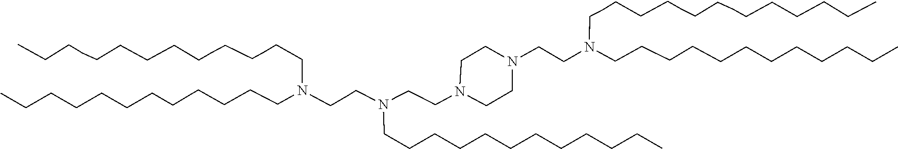

and pharmaceutically acceptable salts thereof.

Other suitable cationic lipids for use in the lipid nanoparticles and compositions and the invention include a cationic lipid having the formula of 14,25-ditridecyl 15,18,21,24-tetraaza-octatriacontane, and pharmaceutically acceptable salts thereof.

Other suitable cationic lipids for use in the lipid nanoparticles and compositions of the invention include the cationic lipids as described in International Patent Publications WO 2013/063468 and WO 2016/205691, each of which are incorporated herein by reference. In some embodiments, the lipid nanoparticles and compositions of the present invention include a cationic lipid of the following formula:

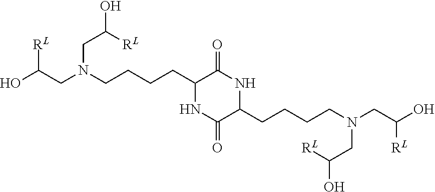

or pharmaceutically acceptable salts thereof, wherein each instance of R^{L} is independently optionally substituted C₆-C₄₀ alkenyl. In certain embodiments, the lipid nanoparticles and compositions of the present invention include a cationic lipid having a compound structure of:

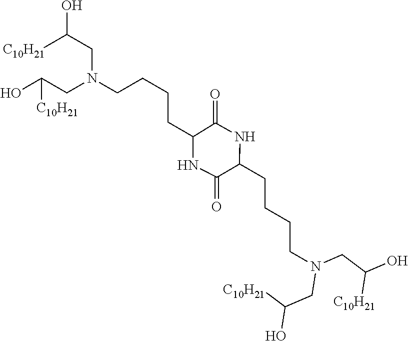

and pharmaceutically acceptable salts thereof. In certain embodiments, the lipid nanoparticles and compositions of the present invention include a cationic lipid having a compound structure of:

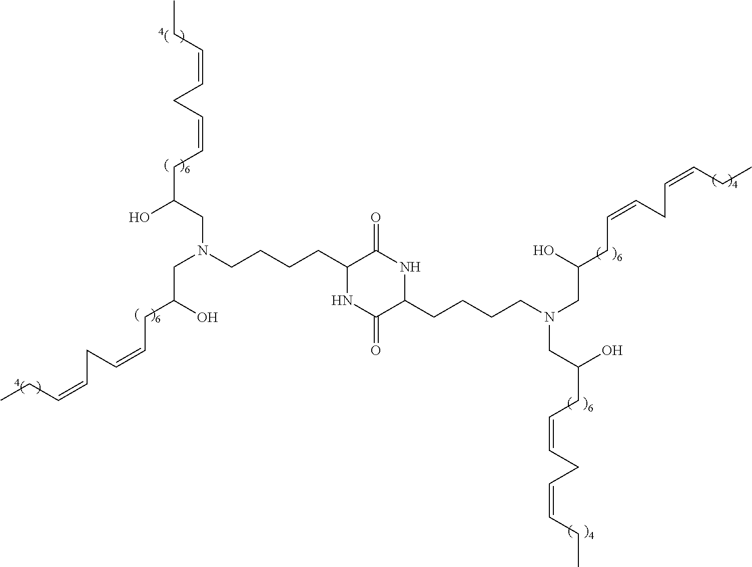

and pharmaceutically acceptable salts thereof. In certain embodiments, the lipid nanoparticles and compositions of the present invention include a cationic lipid having a compound structure of:

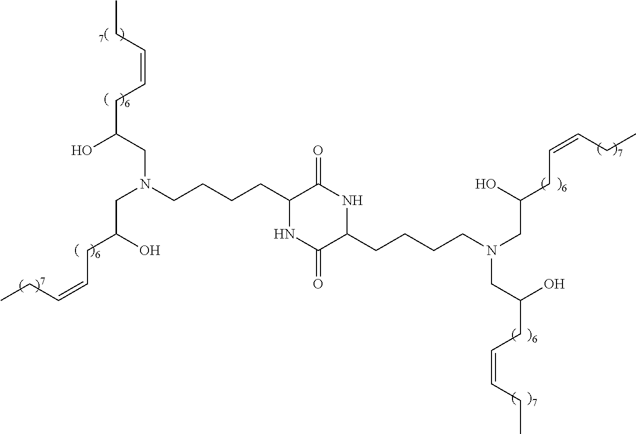

and pharmaceutically acceptable salts thereof. In certain embodiments, the lipid nanoparticles and compositions of the present invention include a cationic lipid having a compound structure of:

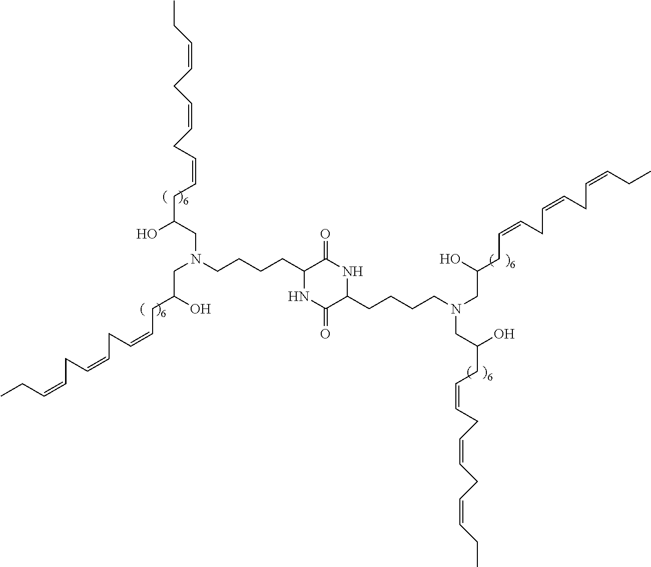

and pharmaceutically acceptable salts thereof.

Other suitable cationic lipids for use in the lipid nanoparticles and compositions of the invention include the cationic lipids as described in International Patent Publication WO 2015/184256, which is incorporated herein by reference. In some embodiments, the lipid nanoparticles and compositions of the present invention include a cationic lipid of the following formula:

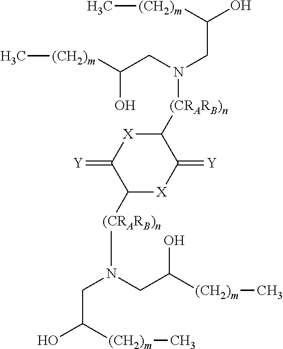

or a pharmaceutically acceptable salt thereof, wherein each X independently is O or S; each Y independently is O or S; each m independently is 0 to 20; each n independently is 1 to 6; each R^{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R^{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the lipid nanoparticles and compositions of the present invention include a cationic lipid, “Target 23”, having a compound structure of:

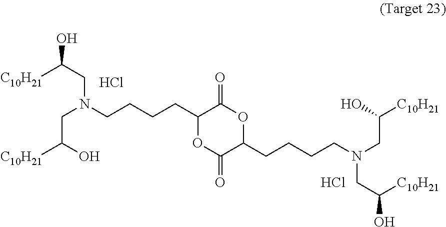

and pharmaceutically acceptable salts thereof.

Other suitable cationic lipids for use in the lipid nanoparticles and compositions of the invention include the cationic lipids as described in International Patent Publication WO 2016/004202, which is incorporated herein by reference. In some embodiments, the lipid nanoparticles and compositions of the present invention include a cationic lipid having the compound structure:

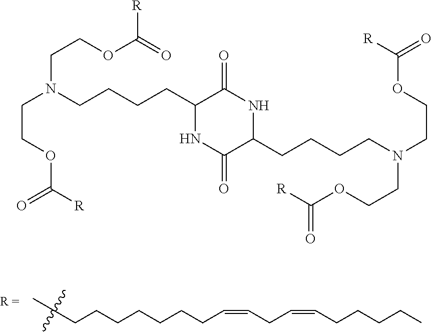

or a pharmaceutically acceptable salt thereof. In some embodiments, the lipid nanoparticles and compositions of the present invention include a cationic lipid having the compound structure:

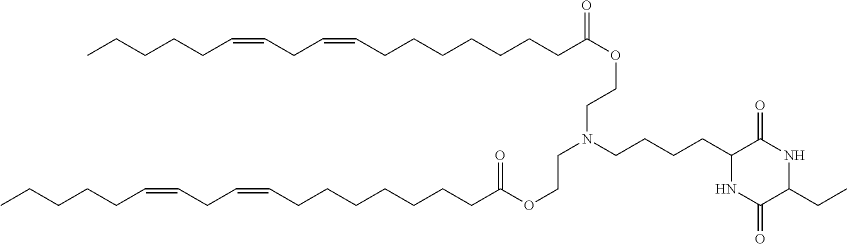 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the lipid nanoparticles and compositions of the present invention include a cationic lipid having the compound structure:

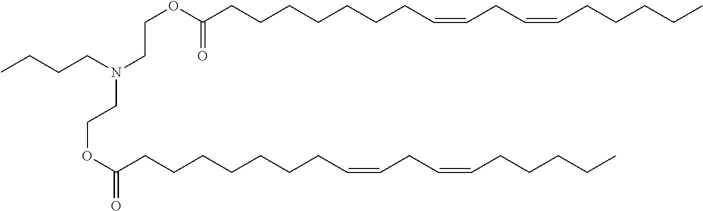 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the lipid nanoparticles and compositions of the present invention include cationic lipids as described in U.S. Provisional Patent Application No. 62/758,179, which is incorporated herein by reference. In some embodiments, the lipid nanoparticles and compositions of the present invention include a cationic lipid of the following formula:

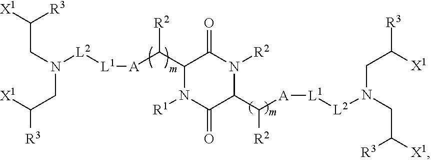

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the lipid nanoparticles and compositions of the present invention include a cationic lipid of the following formula:

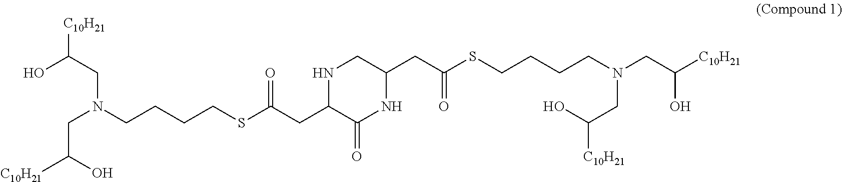

or a pharmaceutically acceptable salt thereof. In some embodiments, the lipid nanoparticles and compositions of the present invention include a cationic lipid of the following formula:

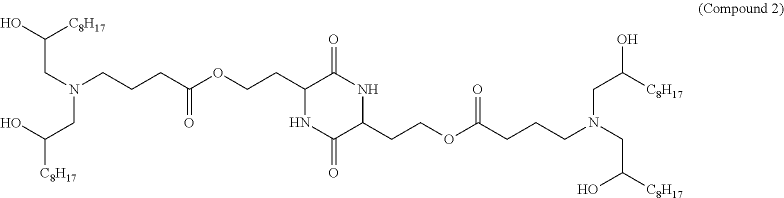

or a pharmaceutically acceptable salt thereof. In some embodiments, the lipid nanoparticles and compositions of the present invention include a cationic lipid of the following formula:

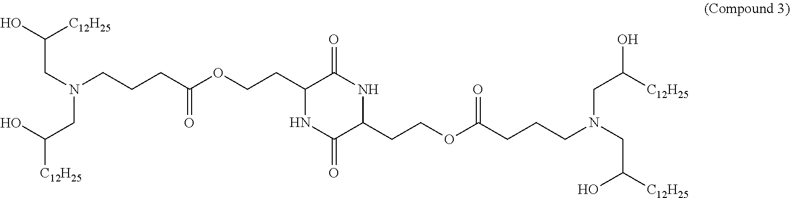

or a pharmaceutically acceptable salt thereof.

Other suitable cationic lipids for use in the lipid nanoparticles and compositions of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the lipid nanoparticles and compositions of the present invention include a cationic lipid having a compound structure of:

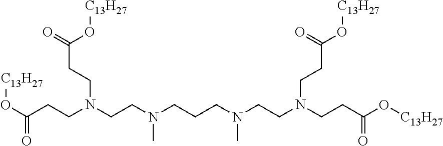

and pharmaceutically acceptable salts thereof.

Other suitable cationic lipids for use in the lipid nanoparticles and compositions of the invention include the cationic lipids as described in International Patent Publication WO 2015/199952, which is incorporated herein by reference. In some embodiments, the lipid nanoparticles and compositions of the present invention include a cationic lipid having the compound structure:

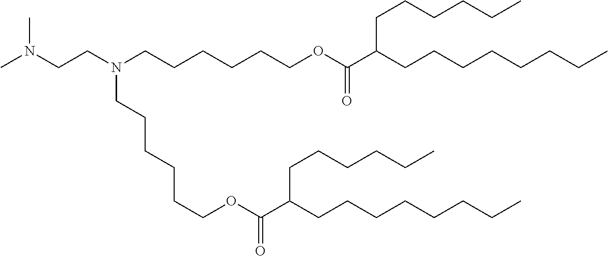 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the lipid nanoparticles and compositions of the present invention include a cationic lipid having the compound structure:

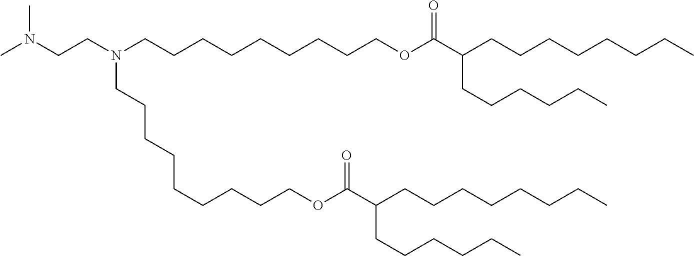 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the lipid nanoparticles and compositions of the present invention include a cationic lipid having the compound structure:

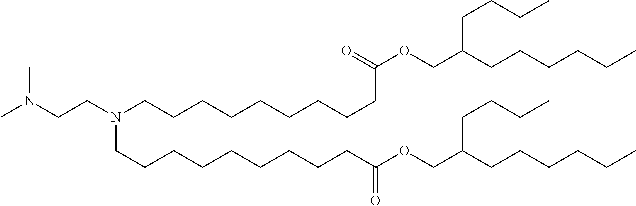 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the lipid nanoparticles and compositions of the present invention include a cationic lipid having the compound structure:

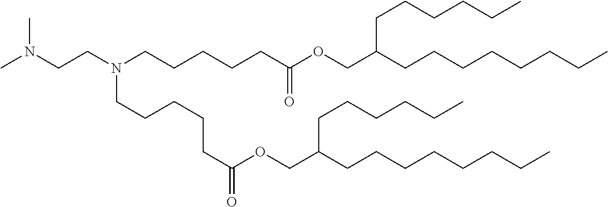 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the lipid nanoparticles and compositions of the present invention include a cationic lipid having the compound structure:

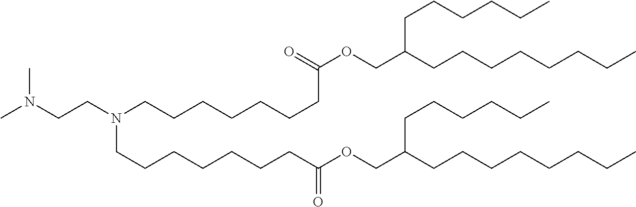 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the lipid nanoparticles and compositions of the present invention include a cationic lipid having the compound structure:

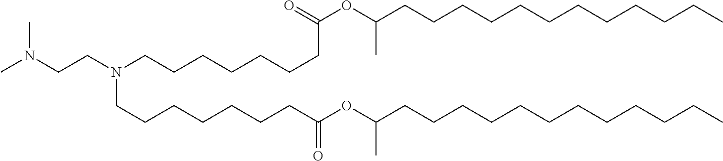 [SMILES: CCCCCCCCCCCCC(C)OC(=O)CCCCCCCN(CCCCCCCC(=O)OC(C)CCCCCCCCCCCC)CCN(C)C]

and pharmaceutically acceptable salts thereof. In some embodiments, the lipid nanoparticles and compositions of the present invention include a cationic lipid having the compound structure:

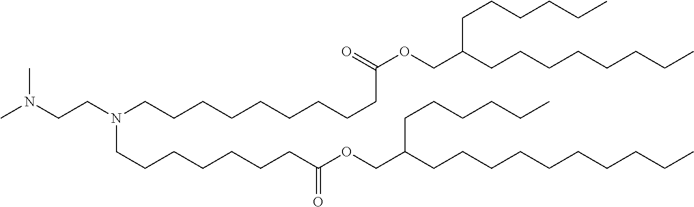 [SMILES: CCCCCCCCCCC(CCCCCC)COC(=O)CCCCCCCN(CCCCCCCCCC(=O)OCC(CCCCCC)CCCCCCCC)CCN(C)C]

and pharmaceutically acceptable salts thereof. In some embodiments, the lipid nanoparticles and compositions of the present invention include a cationic lipid having the compound structure:

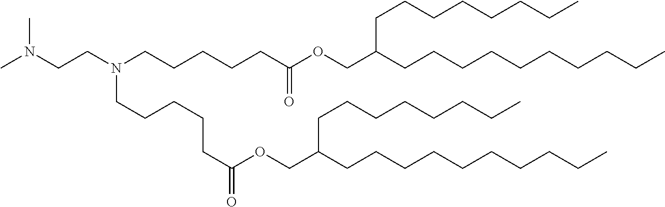 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the lipid nanoparticles and compositions of the present invention include a cationic lipid having the compound structure:

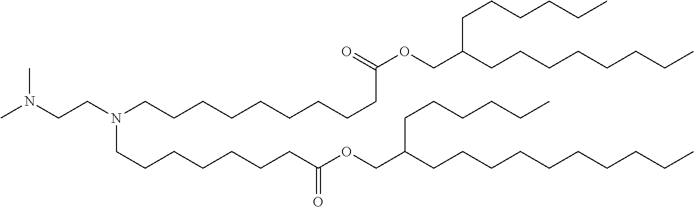 [SMILES: CCCCCCCCCCC(CCCCCC)COC(=O)CCCCCCCN(CCCCCCCCCC(=O)OCC(CCCCCC)CCCCCCCC)CCN(C)C]

and pharmaceutically acceptable salts thereof. In some embodiments, the lipid nanoparticles and compositions of the present invention include a cationic lipid having the compound structure:

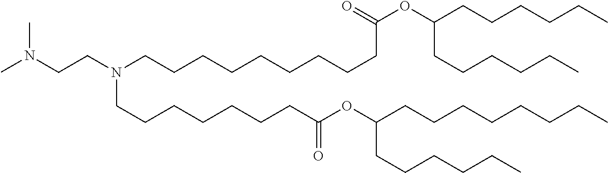 [SMILES: CCCCCCCCC(CCCCCC)OC(=O)CCCCCCCN(CCCCCCCCCC(=O)OC(CCCCCC)CCCCCC)CCN(C)C]

and pharmaceutically acceptable salts thereof. In some embodiments, the lipid nanoparticles and compositions of the present invention include a cationic lipid having the compound structure:

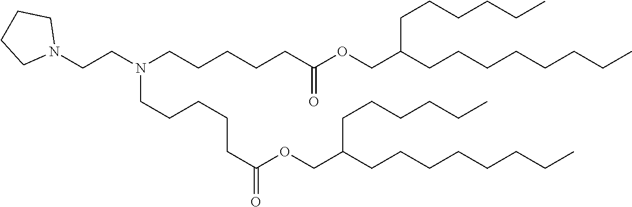 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the lipid nanoparticles and compositions of the present invention include a cationic lipid having the compound structure:

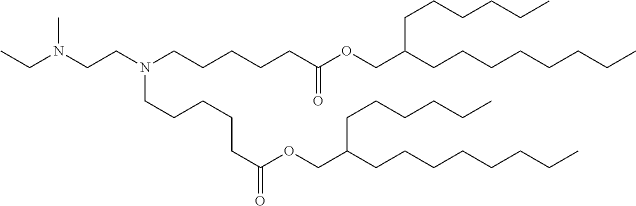 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the lipid nanoparticles and compositions of the present invention include a cationic lipid having the compound structure:

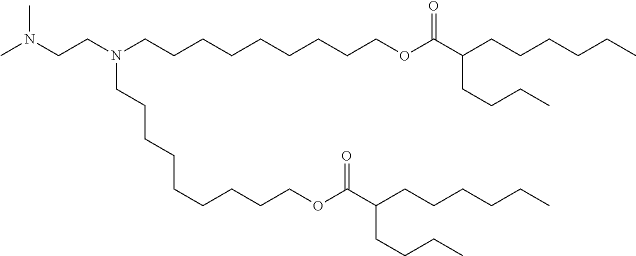 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the lipid nanoparticles and compositions of the invention include the cationic lipids as described in International Patent Publication WO 2017/004143, which is incorporated herein by reference. In some embodiments, the lipid nanoparticles and compositions of the present invention include a cationic lipid having the compound structure:

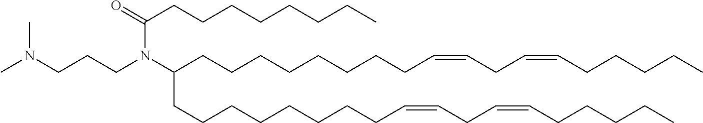 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the lipid nanoparticles and compositions of the present invention include a cationic lipid having the compound structure:

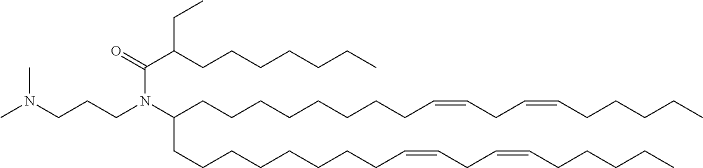 [SMILES: CCCCC/C=C\C/C=C\CCCCCCCCC(CCCCCCCC/C=C\C/C=C\CCCCC)N(CCCN(C)C)C(=O)C(CC)CCCCCCC]

and pharmaceutically acceptable salts thereof. In some embodiments, the lipid nanoparticles and compositions of the present invention include a cationic lipid having the compound structure:

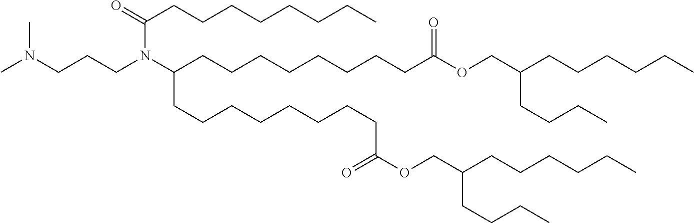 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the lipid nanoparticles and compositions of the present invention include a cationic lipid having the compound structure:

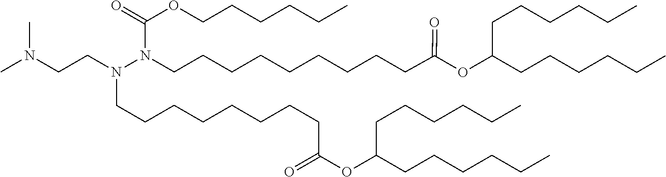 [SMILES: CCCCCCOC(=O)N(CCCCCCCCCC(=O)OC(CCCCCC)CCCCCC)N(CCCCCCCCC(=O)OC(CCCCCC)CCCCCC)CCN(C)C]

and pharmaceutically acceptable salts thereof. In some embodiments, the lipid nanoparticles and compositions of the present invention include a cationic lipid having the compound structure:

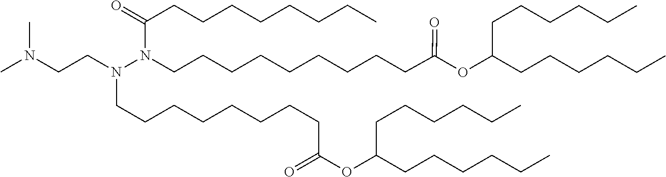 [SMILES: CCCCCCCCC(=O)N(CCCCCCCCCC(=O)OC(CCCCCC)CCCCCC)N(CCCCCCCCC(=O)OC(CCCCCC)CCCCCC)CCN(C)C]

and pharmaceutically acceptable salts thereof. In some embodiments, the lipid nanoparticles and compositions of the present invention include a cationic lipid having the compound structure:

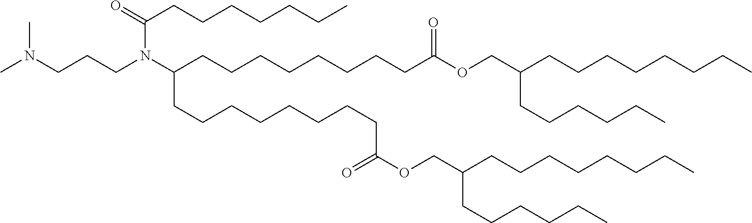 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the lipid nanoparticles and compositions of the present invention include a cationic lipid having the compound structure:

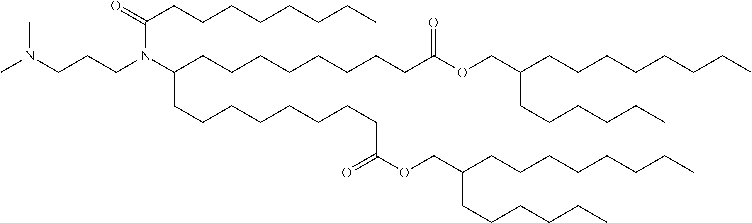 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the lipid nanoparticles and compositions of the present invention include a cationic lipid having the compound structure:

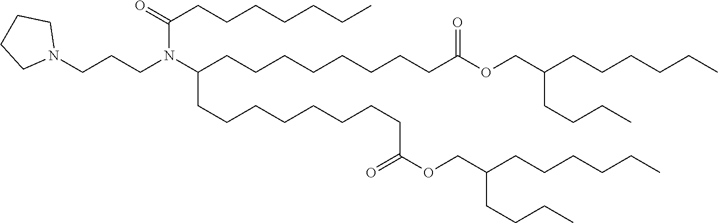 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the lipid nanoparticles and compositions of the present invention include a cationic lipid having the compound structure:

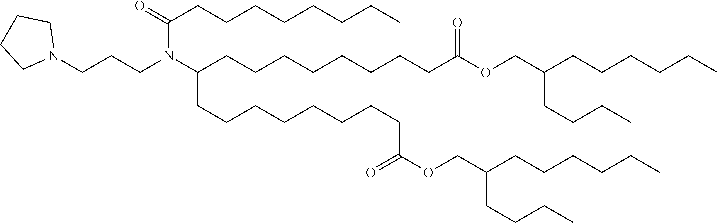 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the lipid nanoparticles and compositions of the present invention include a cationic lipid having the compound structure:

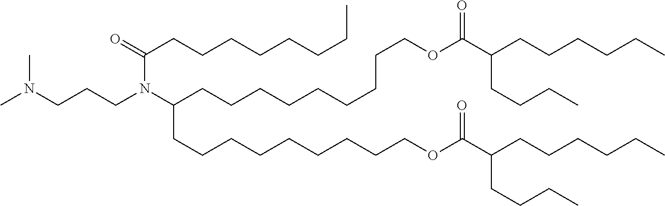 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the lipid nanoparticles and compositions of the present invention include a cationic lipid having the compound structure:

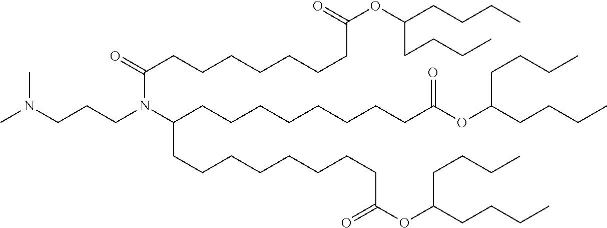 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the lipid nanoparticles and compositions of the present invention include a cationic lipid having the compound structure:

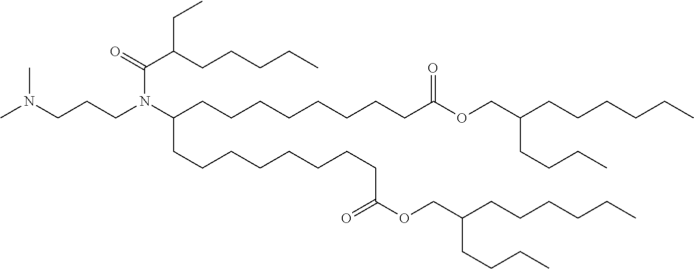 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the lipid nanoparticles and compositions of the present invention include a cationic lipid having the compound structure:

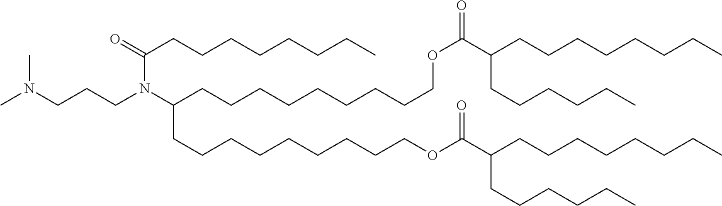 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the lipid nanoparticles and compositions of the present invention include a cationic lipid having the compound structure:

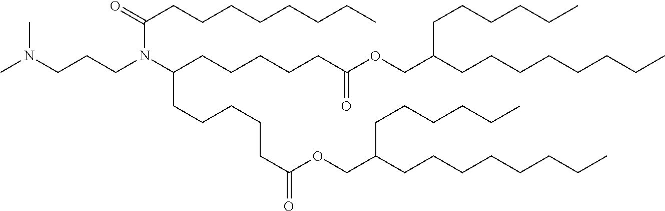 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the lipid nanoparticles and compositions of the present invention include a cationic lipid having the compound structure:

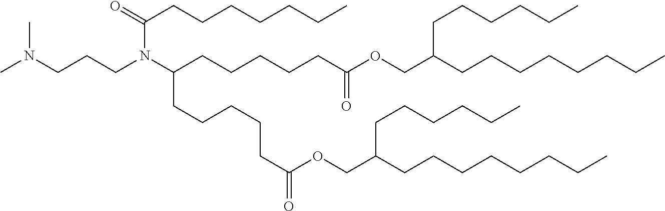 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the lipid nanoparticles and compositions of the present invention include a cationic lipid having the compound structure:

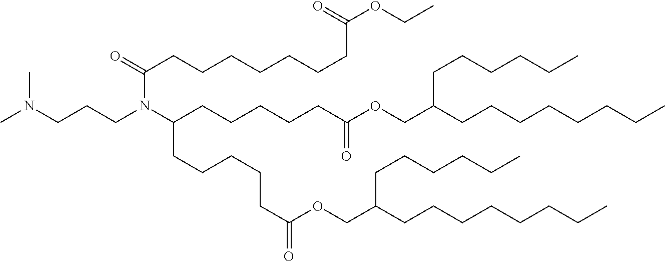 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the lipid nanoparticles and compositions of the present invention include a cationic lipid having the compound structure:

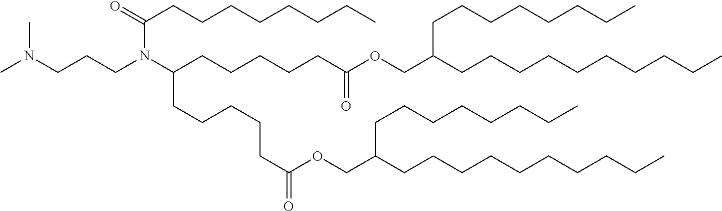 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the lipid nanoparticles and compositions of the invention include the cationic lipids as described in International Patent Publication WO 2017/075531, which is incorporated herein by reference. In some embodiments, the lipid nanoparticles and compositions of the present invention include a cationic lipid of the following formula:

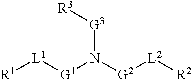

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR—C(═O)—, —C(═O)NR^{a}—, NR—C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene: G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the lipid nanoparticles and compositions of the invention include the cationic lipids as described in International Patent Publication WO 2017/117528, which is incorporated herein by reference. In some embodiments, the lipid nanoparticles and compositions of the present invention include a cationic lipid having the compound structure:

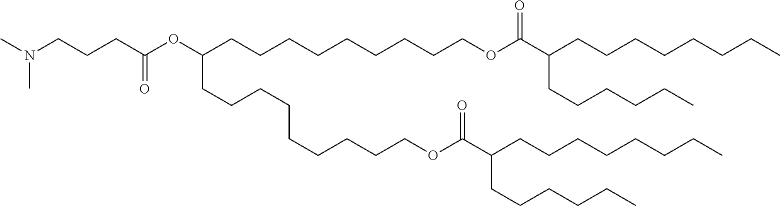 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the lipid nanoparticles and compositions of the present invention include a cationic lipid having the compound structure:

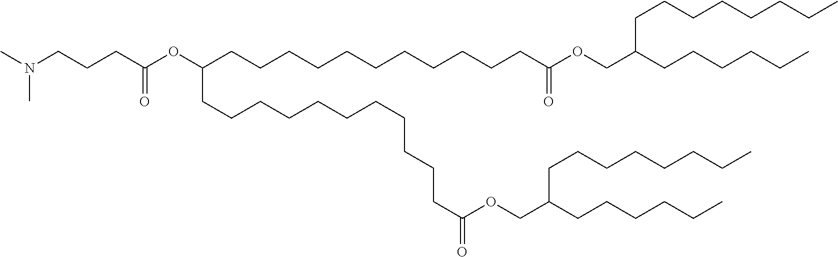 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the lipid nanoparticles and compositions of the present invention include a cationic lipid having the compound structure:

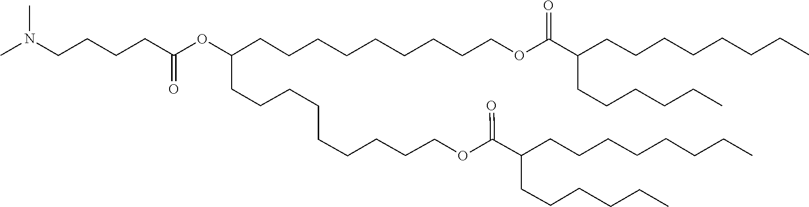 [SMILES: CCCCCCCCCC(CCCCCC)C(=O)OCCCCCCCCCC(CCCCCCCCCOC(=O)C(CCCCCCC)CCCCCCCC)OC(=O)CCCCN(C)C]

and pharmaceutically acceptable salts thereof.

Other suitable cationic lipids for use in the lipid nanoparticles and compositions of the invention include the cationic lipids as described in International Patent Publication WO 2017/049245, which is incorporated herein by reference. In some embodiments, the cationic lipids of the lipid nanoparticles and compositions of the present invention include a compound of one of the following formulas:

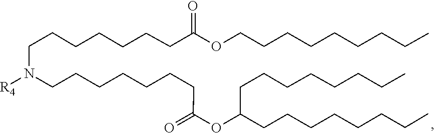

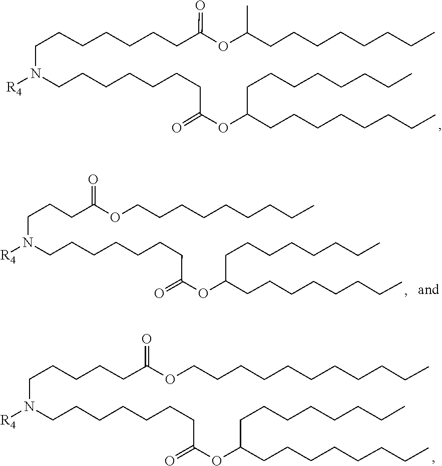

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the lipid nanoparticles and compositions of the present invention include a cationic lipid having a compound structure of

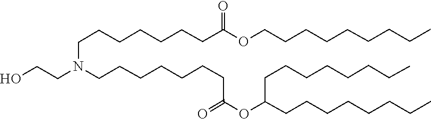 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the lipid nanoparticles and compositions of the present invention include a cationic lipid having a compound structure of:

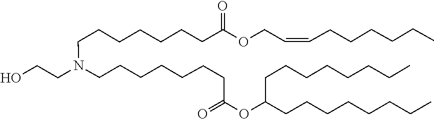 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the lipid nanoparticles and compositions of the present invention include a cationic lipid having a compound structure of:

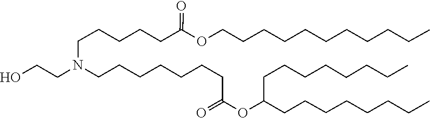 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the lipid nanoparticles and compositions of the present invention include a cationic lipid having a compound structure of:

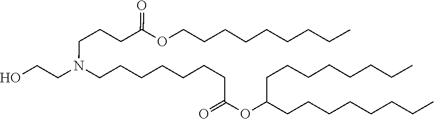 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the lipid nanoparticles and compositions of the invention include the cationic lipids as described in International Patent Publication WO 2017/173054 and WO 2015/095340, each of which is incorporated herein by reference. In certain embodiments, the lipid nanoparticles and compositions of the present invention include a cationic lipid having a compound structure of:

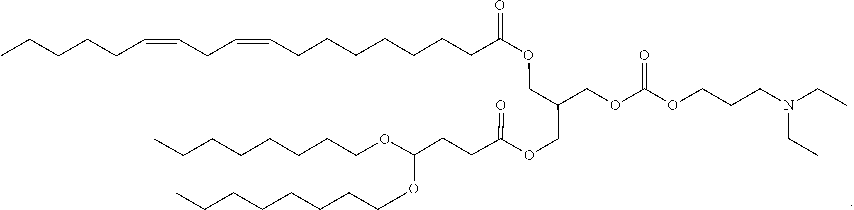 [SMILES: CCCCC/C=C\C/C=C\CCCCCCCC(=O)OCC(COC(=O)CCC(OCCCCCCCC)OCCCCCCCCC)COC(=O)OCCCN(CC)CC]

and pharmaceutically acceptable salts thereof. In certain embodiments, the lipid nanoparticles and compositions of the present invention include a cationic lipid having a compound structure of:

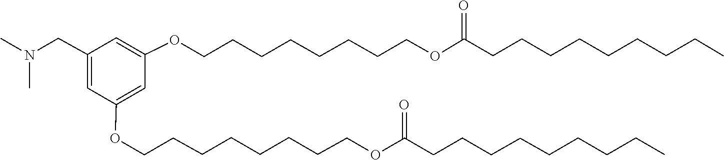 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the lipid nanoparticles and compositions of the present invention include a cationic lipid having a compound structure of:

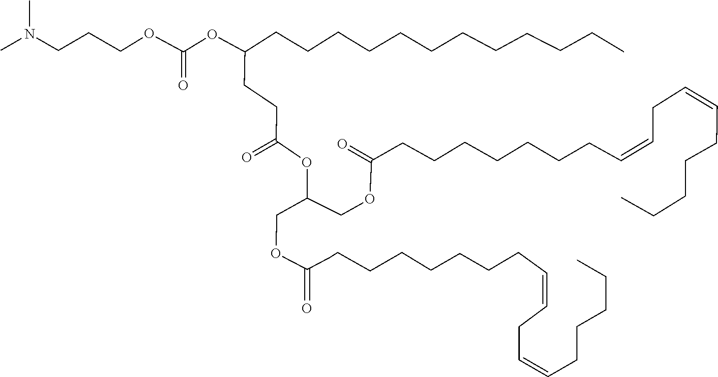 [SMILES: CCCCC/C=C\C/C=C\CCCCCCCCC(=O)OCC(COC(=O)CCCCCCC/C=C\C/C=C\CCCCC)OC(=O)CCC(CCCCCCCCCCCCCC)OC(=O)OCCCN(C)C]

and pharmaceutically acceptable salts thereof. In certain embodiments, the lipid nanoparticles and compositions of the present invention include a cationic lipid having a compound structure of:

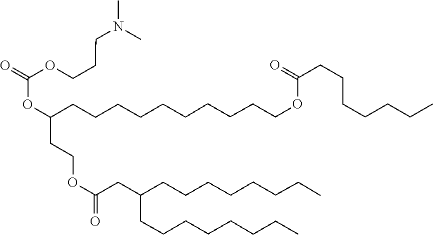 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the lipid nanoparticles and compositions of the present invention include cleavable cationic lipids as described in International Patent Publication WO 2012/170889, which is incorporated herein by reference. In some embodiments, the lipid nanoparticles and compositions of the present invention include a cationic lipid of the following formula:

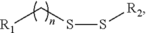

wherein R₁ is selected from the group consisting of imidazole, guanidinium, amino, imine, enamine, an optionally-substituted alkyl amino (e.g., an alkyl amino such as dimethylamino) and pyridyl: wherein R₂ is selected from the group consisting of one of the following two formulas:

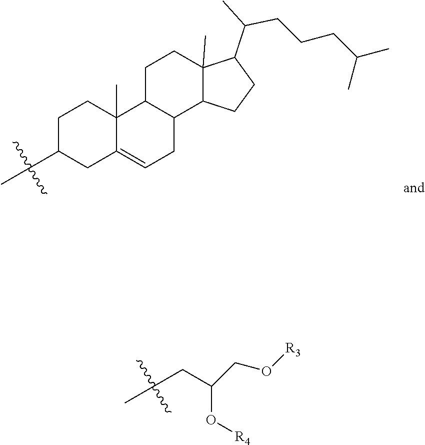

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the lipid nanoparticles and compositions of the present invention include a cationic lipid, “HGT4001”, having a compound structure of:

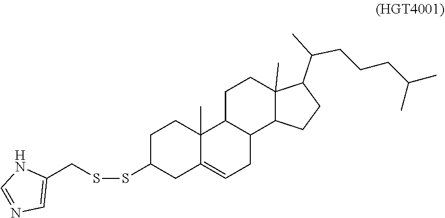 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the lipid nanoparticles and compositions of the present invention include a cationic lipid, “HGT4002” (also referred to herein as “Guan-SS-Chol”), having a compound structure of:

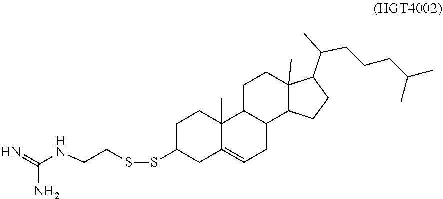 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the lipid nanoparticles and compositions of the present invention include a cationic lipid, “HGT4003”, having a compound structure of:

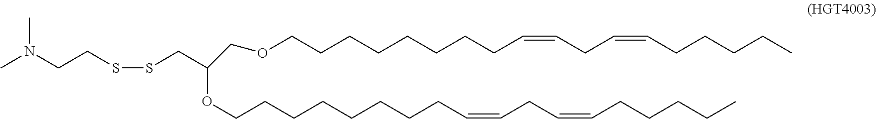 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the lipid nanoparticles and compositions of the present invention include a cationic lipid, “HGT4004”, having a compound structure of:

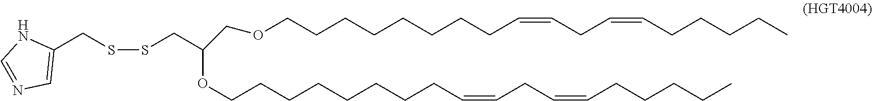 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the lipid nanoparticles and compositions of the present invention include a cationic lipid “HGT4005”, having a compound structure of:

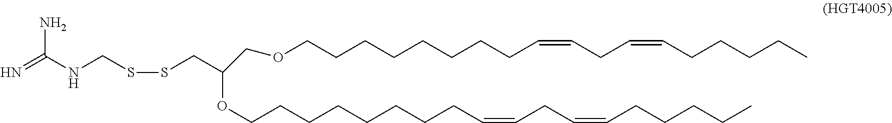 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the lipid nanoparticles and compositions of the present invention include cleavable cationic lipids as described in U.S. Provisional Patent Application No. 62/672,194, filed May 16, 2018, and incorporated herein by reference. In certain embodiments, the lipid nanoparticles and compositions of the present invention include a cationic lipid that is any of general formulas or any of structures (1a)-(21a) and (1b)-(21b) and (22)-(237) described in U.S. Provisional Patent Application No. 62/672,194.

In certain embodiments, the lipid nanoparticles and compositions of the present invention include a cationic lipid that has a structure according to Formula (I′),

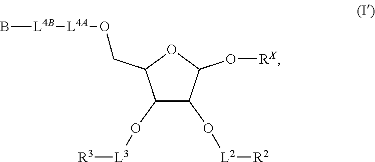

wherein:
   R^{X} is independently —H, -L¹-R¹, or -L^{5A}-L^{5B}-B′;
   each of L¹, L², and L is independently a covalent bond, —C(O)—, —C(O)O—, —C(O)S—, or —C(O)NR^{L}—;
   each L^{4A} and L^{5A} is independently —C(O)—, —C(O)O—, or —C(O)NR^{L}—;
   each L^{4B} and L^{5B} is independently C₁-C₂₀ alkylene; C₂-C₂₀ alkenylene; or C₂-C₂₀ alkynylene;
   each B and B′ is NR⁴R⁵ or a 5- to 10-membered nitrogen-containing heteroaryl;
   each R¹, R², and R³ is independently C₆-C₃₀ alkyl, C₆-C₃₀ alkenyl, or C₆-C₃₀ alkynyl;
   each R⁴ and R⁵ is independently hydrogen, C₁-C₁₀ alkyl; C₂-C₁₀ alkenyl; or C₂-C₁₀ alkynyl; and
   each R^{L} is independently hydrogen, C₁-C₂₀ alkyl, C₂-C₂₀ alkenyl, or C₂-C₂₀ alkynyl.

In certain embodiments, the lipid nanoparticles and compositions of the present invention include a cationic lipid that is Compound (139) of 62/672,194, having a compound structure of:

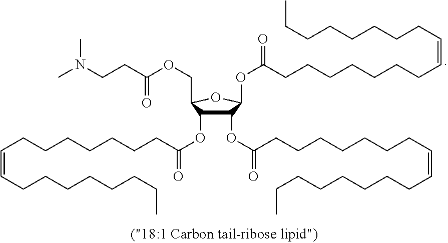

In some embodiments, the lipid nanoparticles and compositions of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the lipid nanoparticles and compositions of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. No. 5,171,678: U.S. Pat. No. 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the lipid nanoparticles and compositions of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-15′-(cholest-5-en-3-beta-oxy)-3′-oxapentoxy)-3-dimethyl-1-(cis,cis-9′, 1-2′-octadecadienoxy)propane (“CpLinDMA”); N,N-dimethyl-3,4-dioleyloxybenzylamine (“DMOBA”): 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxyloctyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-1(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety. In some embodiments, the cationic lipid suitable for the lipid nanoparticles and compositions of the present invention is selected from 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the cationic lipid has a structure according to Formula (IIIE):

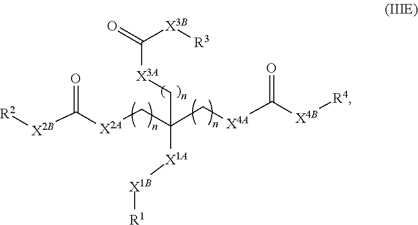

or a pharmaceutically acceptable salt thereof, wherein
each n is independently 0 or 1;
X^{1A} is independently O or NR^{1A};
R^{1A} is H or C₁-C₆ alkyl;
X^{1B} is a covalent bond, C(O), CH₂CO₂, or CH₂C(O);
one of X^{2A} and X^{2B} is O and the other is a covalent bond;
one of X^{3A} and X^{3B} is O and the other is a covalent bond;
one of X^{4A} and X^{4B} is O and the other is a covalent bond;
R¹ is independently L¹-B¹, C₆-C₃₀ alkyl, C₆-C₃₀ alkenyl, or C₆-C₃₀ alkynyl;
R² is independently L²-B². C₆-C₃₀ alkyl, C₆-C₃₀ alkenyl, C₆-C₃₀ alkynyl;
R³ is independently L³-B³, C₆-C₃₀ alkyl, C₆-C₃₀ alkenyl, C₆-C₃₀ alkynyl;
R⁴ is independently L⁴-B⁴, C₆-C₃₀ alkyl, C₆-C₃₀ alkenyl, C₆-C₃₀ alkynyl;
L¹, L², L³, and L⁴ are each independently C₁-C₃₀ alkylene; C₂-C₃₀ alkenylene; or C₂-C₃₀ alkynylene;
each of B¹, B², B³, and B⁴ is independently an ionizable nitrogen-containing group, and
wherein the cationic lipid comprises at least one ionizable nitrogen-containing group.

In some embodiments, the cationic lipids has a structure according to Formula (IIIF):

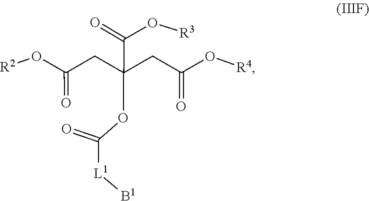

or a pharmaceutically acceptable salt thereof, wherein
B¹ is an ionizable nitrogen-containing group;
L¹ is C₁-C₁₀ alkylene:
each of R², R³, and R⁴ is independently C₆-C₃₀ alkyl, C₆-C₃₀ alkenyl, C₆-C₃₀ alkynyl.

In some embodiments, the cationic lipid has a structure according to Formula (IIIG):

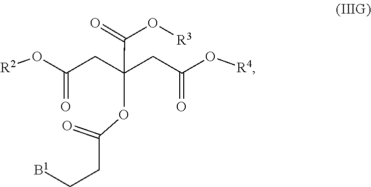

or a pharmaceutically acceptable salt thereof, wherein
B¹ is an ionizable nitrogen-containing group;
each of R², R³, and R⁴ is independently C₆-C₃₀ alkyl, C₆-C₃₀ alkenyl, C₆-C₃₀ alkynyl.

In some embodiments, each of R², R³, and R⁴ in the cationic lipid according to any of Formulae IIIE-IIIG is independently C₆-C₁₂ alkyl substituted by —O(CO)R⁵ or —C(O)OR⁵, wherein R⁵ is unsubstituted C₆-C₁₄ alkyl. In some embodiments, each of R², R³, and R⁴ in the cationic lipid according to any of Formulae IIIE-IIIG is independently:

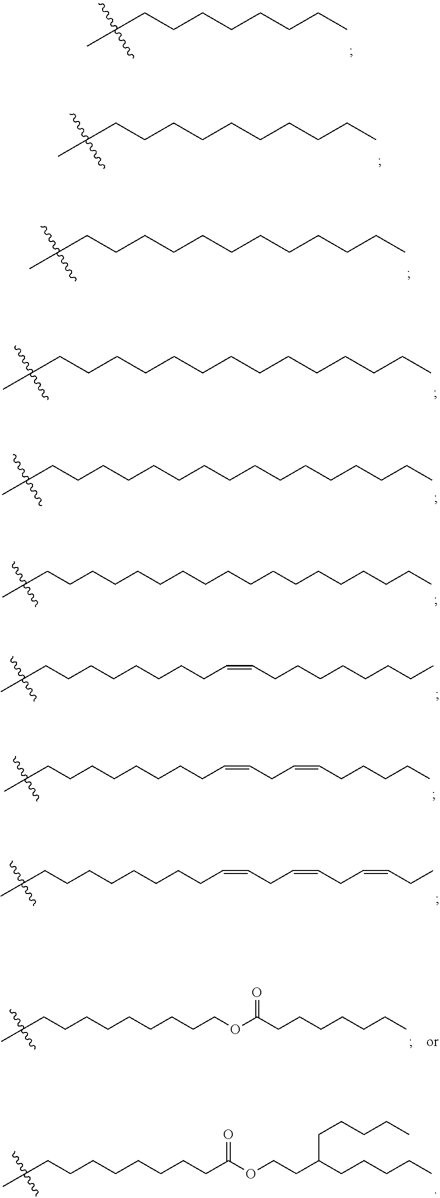

In some embodiments, B¹ in the cationic lipid according to any of Formulae IIIE-IIIG is
d) NH₂, guanidine, amidine, a mono- or dialkylamine, 5- to 6-membered nitrogen-containing heterocycloalkyl, or 5- to 6-membered nitrogen-containing heteroaryl;
e)

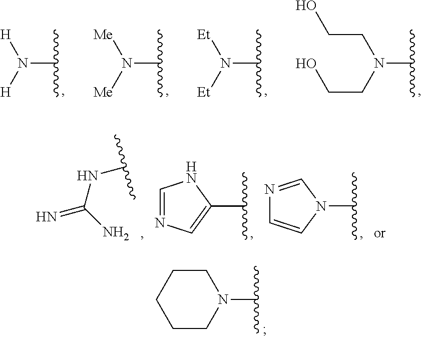

f)

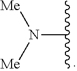 [SMILES: *N(C)C]

In some embodiments, L¹ is in the cationic lipid according to any of Formulae IIIE-IIIG C₁-alkylene.

In some embodiments, the cationic lipid suitable for the lipid nanoparticles and compositions of the present invention is TL1-04D-DMA, having a compound structure of:

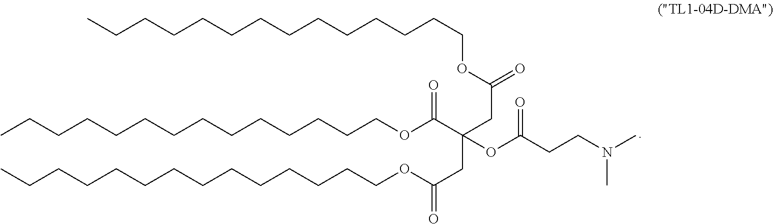 [SMILES: CCCCCCCCCCCCCCOC(=O)CC(CC(=O)OCCCCCCCCCCCCCC)(OC(=O)CCN(C)C)C(=O)OCCCCCCCCCCCCCC]

In some embodiments, the cationic lipid suitable for the lipid nanoparticles and compositions of the present invention is GL-TES-SA-DME-E18-2, having a compound structure of:

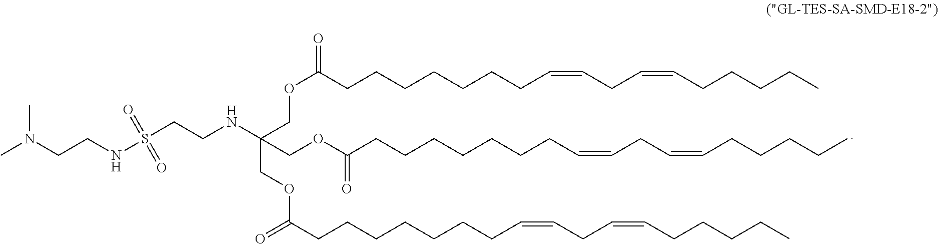 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

In some embodiments, the cationic lipid suitable for the lipid nanoparticles and compositions of the present invention is SY-3-E14-DMAPr, having a compound structure of:

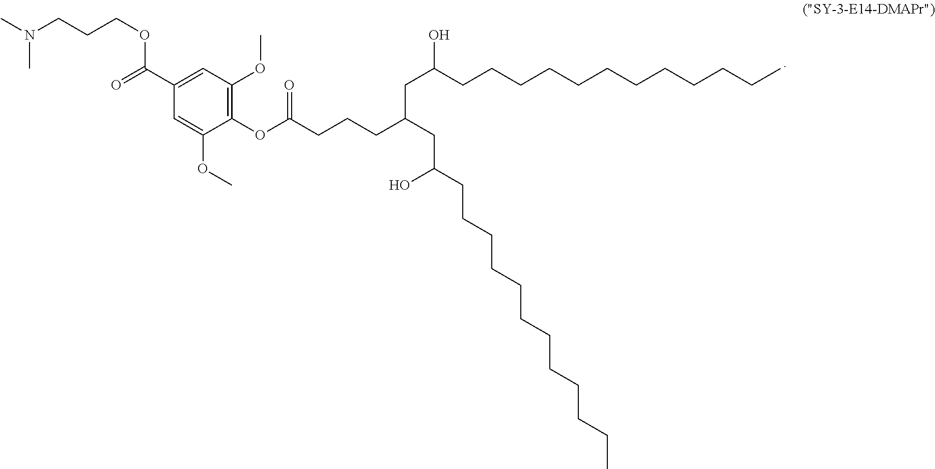 [SMILES: CCCCCCCCCCCCC(O)CC(CCCC(=O)Oc1c(OC)cc(C(=O)OCCCN(C)C)cc1OC)CC(O)CCCCCCCCCCCC]

In some embodiments, the cationic lipid suitable for the lipid nanoparticles and compositions of the present invention is TL1-01D-DMA, having a compound structure of:

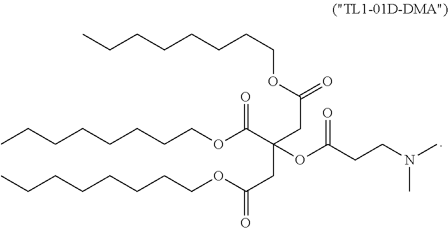 [SMILES: CCCCCCCCOC(=O)CC(CC(=O)OCCCCCCCC)(OC(=O)CCN(C)C)C(=O)OCCCCCCCC]

In some embodiments, the cationic lipid suitable for the lipid nanoparticles and compositions of the present invention is TL1-10D-DMA, having a compound structure of:

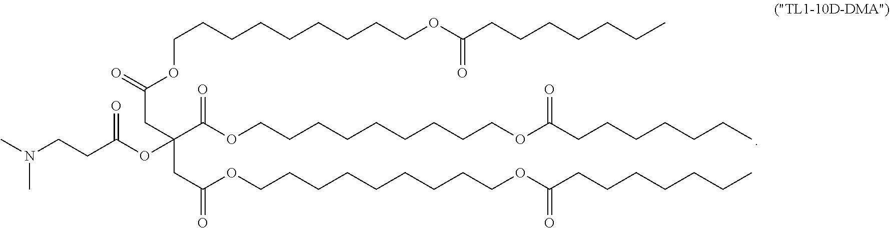 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

In some embodiments, the cationic lipids suitable for the lipid nanoparticles and compositions of the present invention is GL-TES-SA-DMP-E18-2, having a compound structure of:

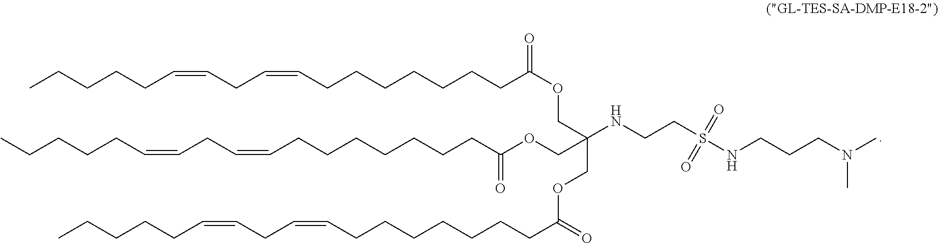 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

In some embodiments, the cationic lipid suitable for the lipid nanoparticles and compositions of the present invention is HEP-E4-E10, having a compound structure of:

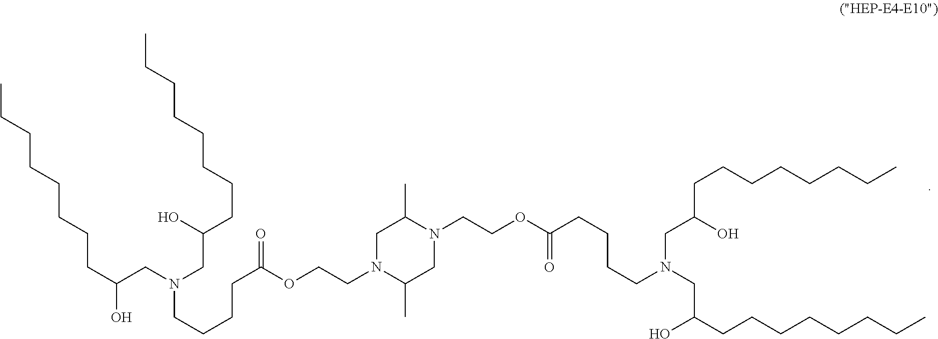 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

In some embodiments, the cationic lipid suitable for the lipid nanoparticles and compositions of the present invention is HEP-E3-E10, having a compound structure of:

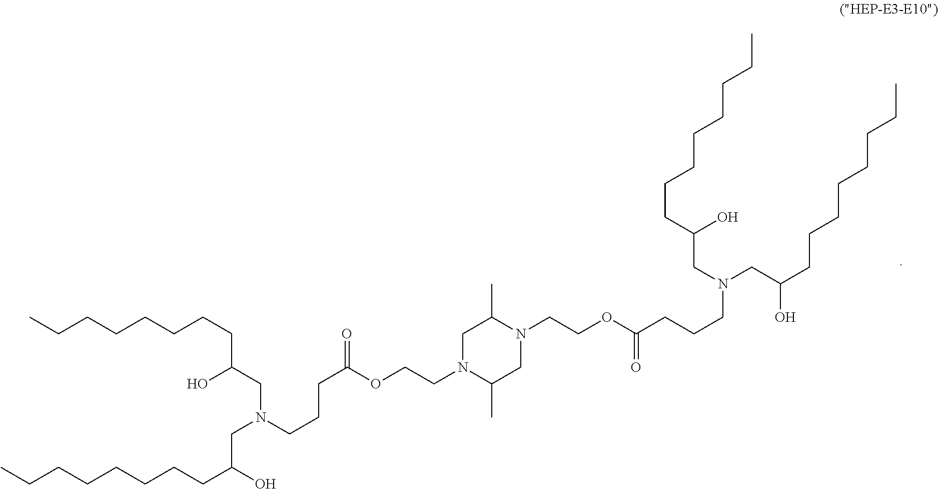 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

In some embodiments, the cationic lipid suitable for the lipid nanoparticles and compositions of the present invention is SI-4-E14-DMAPr, having a compound structure of:

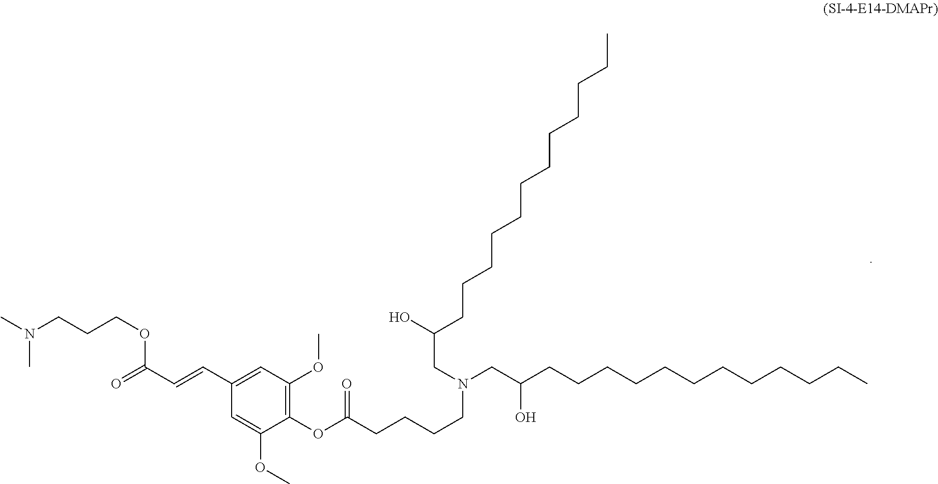 [SMILES: CCCCCCCCCCCCC(O)CN(CCCCC(=O)Oc1c(OC)cc(/C=C/C(=O)OCCCN(C)C)cc1OC)CC(O)CCCCCCCCCCCC]

In some embodiments, the cationic lipid suitable for the lipid nanoparticles and compositions of the present invention is SY-010, having a compound structure of:

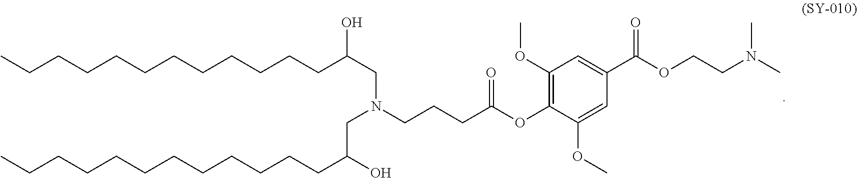 [SMILES: CCCCCCCCCCCCC(O)CN(CCCC(=O)Oc1c(OC)cc(C(=O)OCCN(C)C)cc1OC)CC(O)CCCCCCCCCCCC]

In some embodiments, the cationic lipid suitable for the lipid nanoparticles and compositions of the present invention is SY-011, having a compound structure of:

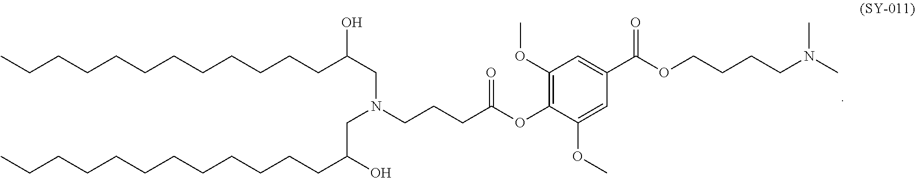 [SMILES: CCCCCCCCCCCCC(O)CN(CCCC(=O)Oc1c(OC)cc(C(=O)OCCCCN(C)C)cc1OC)CC(O)CCCCCCCCCCCC]

In some embodiments, the cationic lipid suitable for the lipid nanoparticles and compositions of the present invention is TL1-12D-DMA, having a compound structure of:

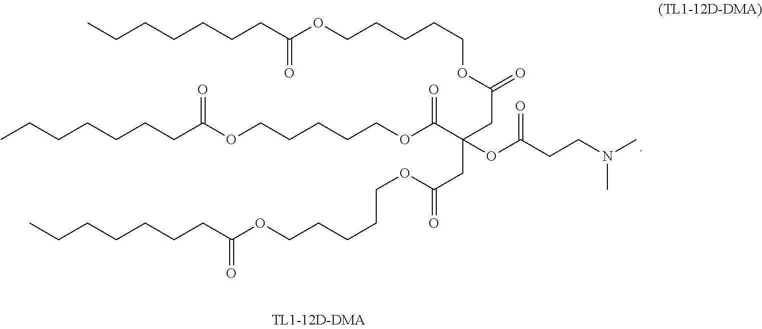 [SMILES: CCCCCCCC(=O)OCCCCCOC(=O)CC(CC(=O)OCCCCCOC(=O)CCCCCCC)(OC(=O)CCN(C)C)C(=O)OCCCCCOC(=O)CCCCCCC]

The molar ratio of the cationic lipid in a lipid nanoparticle in accordance with the invention is 41%-70%. In some embodiments, the molar ratio of the cationic lipid is 45%-70%. In some embodiments, the molar ratio of the cationic lipid is 45%-65%. In some embodiments, the molar ratio of the cationic lipid is 50%-70%. In some embodiments, the molar ratio of the cationic lipid is 50%-65%. In particular embodiments, the molar ratio of the cationic lipid is 50%-60%. A molar ratio of 50%-60% for the cationic lipid in a lipid nanoparticle in accordance with the invention was found to result in high encapsulation efficiency, which was maintained before and after nebulization. Without wishing to be bound by any particular theory, using the lowest possible amount of cationic lipid may be particularly advantageous because it reduces the possibility for encountering toxicity and adverse reactions during therapy with a lipid nanoparticle of the invention. Accordingly, in one specific embodiment, the molar ratio of the cationic lipid is about 50%. In another specific embodiment, the molar ratio of the cationic lipid is about 55%. In yet a further specific embodiment, the molar ratio of the cationic lipid is about 60%.

In some embodiments, the cationic lipid in a lipid nanoparticle in accordance with the invention is any of the cationic lipids disclosed in PCT/US21/25128, which is incorporated herein by reference.

### Non-Cationic Lipid

As used herein, the phrase “non-cationic lipid” refers to neutral, zwitterionic or anionic lipid. Specific non-cationic lipids which are suitable for use in the lipid nanoparticles of the invention are distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-dilinoleoyl-sn-glycero-3-phosphoethanolamine (DLoPE), 1,2-dilauroyl-sn-glycero-3-phosphorylethanolamine (DLPE), 1,2-dilauroyl-sn-glycero-3-phosphocholine (DLPC), 1,2-dioleoyl-sn-glycero-3-phospho-L-serine (DOPS), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dierucoyl-sn-glycero-3-phosphoethanolamine (DEPE), phosphatidylserine, sphingolipids, cerebrosides, gangliosides, 16-O-monomethyl PE, 16-O-dimethyl PE, 18-1-trans PE, l-stearoyl-2-olcoyl-phosphatidyethanolamine (SOPE), 1,2-dimyristelaidoyl-sn-glycero-3-phosphocholine (14:1 PC), 1,2-dipalmitelaidoyl-sn-glycero-3-phosphocholine (16:1PC), or 1,2-dimyristoyl-sn-glycero-3-phosphocholine (DMPC).

Naturally occurring zwitterionic lipids are typically phospholipids and can be broadly grouped into two classes: (i) phospholipids that comprise an ethanolamine substituent in their headgroup (also referred to as PE lipids); and (ii) phospholipids that comprise a choline substituent in their headgroup (also referred to as PC lipids). The inventors found that lipid nanoparticles comprising a zwitterionic phospholipids with an ethanolamine substituent in their headgroup as their non-cationic lipid generally show greater in vivo potency post-nebulization than phospholipids that comprise a choline substituent in their headgroup.

Accordingly, in some embodiments, lipid nanoparticles in accordance with the invention include a PE lipid as the non-cationic lipid component. In some embodiments, lipid nanoparticles in accordance with the invention include DOPE, DLoPE, DMPE, or DLPE as the non-cationic lipid component. In particular embodiments, lipid nanoparticles in accordance with the invention include DOPE as the non-cationic lipid component. In other embodiments, lipid nanoparticles in accordance with the invention include DEPE as the non-cationic lipid component.

In some embodiments, lipid nanoparticles in accordance with the invention include DOPC, DPPC or DSPC as the non-cationic lipid component. In particular embodiments, lipid nanoparticles in accordance with the invention include DOPC as the non-cationic lipid component.

The molar ratio of the non-cationic lipid in a lipid nanoparticle in accordance with the invention is 9%-18%. In some embodiments, the molar ratio of the non-cationic lipid is 9%-15%. In particular embodiments, the molar ratio of the non-cationic lipid is 10%-15%. Including a non-cationic lipid at a molar ratio falling within this range in a lipid nanoparticle was found to result in particularly effective nebulization properties, as illustrated in the examples. Moreover, the reduction in the total amount of non-cationic lipid and the associated reduction in the overall lipid content of the lipid nanoparticle did not negatively impact encapsulation efficiency, both before and after nebulization. In addition, lipid nanoparticles with a molar ratio of the non-cationic lipid of 10%-15% were found to be particularly potent in inducing in vivo expression of the protein encoded by the mRNA encapsulated within them. Accordingly, in a specific embodiment, the molar ratio of the non-cationic lipid is about 15%. In another specific embodiment, the molar ratio of the non-cationic lipid is about 12.5%. In yet a further specific embodiment, the molar ratio of the non-cationic lipid is about 10%.

### PEG-Modified Lipids

The lipid nanoparticles of the invention include a polyethylene glycol (PEG)-modified lipid. In one embodiment, the PEG-modified lipid is a PEG-modified phospholipid or other derivatized lipid such as a derivatized ceramide (PEG-CER), e.g., N-Octanoyl-Sphingosine-1-[Succinyl(Methoxy Polyethylene Glycol)-2000] (C8 PEG-2000 ceramide).

PEG-modified lipids typically include a polyethylene glycol chain of up to 5 kDa in length covalently attached to a lipid with alkyl chain(s) of C₆-C₂₀ length. Particularly useful are PEG-modified exchangeable lipids having shorter acyl chains (e.g., C₁₄ or C₁₈). In some embodiments, a PEG-modified (or PEGylated lipid) is a PEGylated cholesterol. Lipid nanoparticles in accordance with the invention typically include a PEG-modified lipid such as 1,2-dimyristoyl-rac-glycero-3-methoxypolyethylene glycol-2000 (DMG-PEG2K). Alternatively, they may include [(polyethylene glycol)-2000]-N,N-ditetradecylacetamide or DSPE-PEG2K-COOH.

The inclusion of such PEG-modified lipids prevents complex aggregation (e.g., during storage and nebulization). Their presence may also increase the mucopenetrating capacity of the lipid nanoparticles of the invention, once they have been delivered into the lung.

In some embodiments, the molar ratio of the PEG-modified lipid in a lipid nanoparticle in accordance with the invention is 2%-6%, e.g., 3%-6% or 4%-6%. In particular embodiments, the molar ratio of the PEG-modified lipid is 3%-5%. Lipid nanoparticles with a molar ratio of the PEG-modified lipid falling within this range have been found to be particularly effective in delivering their mRNA cargo through the mucus layer to the underlying epithelium of the lungs. Accordingly, in one specific embodiment, the molar ratio of the PEG-modified lipid is about 5%. In another specific embodiment, the molar ratio of the PEG-modified lipid is about 4%. In yet a further specific embodiment, the molar ratio of the PEG-modified lipid is about 3%.

For certain applications, lipid nanoparticles in which the PEG-modified lipid component constitutes about 5% of the total lipids by molar ratio have been found to be particularly suitable.

### Cholesterol and Cholesterol Analogues

Lipid nanoparticles in accordance with the invention typically include cholesterol as one of the four lipids of their lipid component. In some embodiments, it may be advantageous to use a cholesterol analogue in place of cholesterol. As used herein, the term “cholesterol analogue” encompasses compounds that have a similar structure to cholesterol but differ in one or more atoms, functional groups and/or substructures. In some embodiments, the cholesterol analogue is a functional analogue of cholesterol, for example, it has similar physical, chemical, biochemical and/or pharmacological properties to cholesterol. Examples of cholesterol analogues include but are not limited to: β-sitosterol, stigmastanol, campesterol, fucosterol, stigmasterol, and dexamethasone.

Both β-sitosterol and stigmastanol have been found to be particularly suitable in the preparation of lipid nanoparticles with improved nebulization properties. Accordingly, in one specific embodiment, the cholesterol analogue used in place of cholesterol in a lipid nanoparticle of the invention is β-sitosterol. In another specific embodiment, the cholesterol analogue used in place of cholesterol in a lipid nanoparticle of the invention is stigmastanol.

The molar ratio of the cholesterol or cholesterol analogue in a lipid nanoparticle in accordance with the invention is 9%-48%. Cholesterol or a cholesterol analogue can be used as a “filler lipid” in a lipid nanoparticle. In some embodiments, the molar ratio of the cholesterol or cholesterol analogue is 10%-45%. In particular embodiments, the molar ratio of the cholesterol or cholesterol analogue is 10%-30%. More typically, about 25% to 30% (by molar ratio) of the lipid component of a lipid nanoparticle in accordance with the invention is cholesterol or a cholesterol analogue. Accordingly, in one particular embodiment, the molar ratio of the cholesterol or cholesterol analogue is 25%-30%. In a specific embodiment, the molar ratio of the cholesterol or cholesterol analogue is about 25%. In another specific embodiment, the molar ratio of the cholesterol or cholesterol analogue is about 30%.

### Exemplary Lipid Nanoparticle Formulations

A lipid nanoparticle of the present invention may include any of the cationic lipids, non-cationic lipids, cholesterol lipids, and PEG-modified lipids described herein.

Cationic lipids particularly suitable for inclusion in such lipid nanoparticles include GL-TES-SA-DME-E 18-2, TL1-01D-DMA, SY-3-E14-DMAPr, TL1-10D-DMA, GL-TES-SA-DMP-E18-2. HEP-E4-E10, HEP-E3-E10, and TL1-04D-DMA. These cationic lipids have been found to be particularly suitable for use in lipid nanoparticles that are administered through pulmonary delivery via nebulization. Amongst these, HEP-E4-E10, HEP-E3-E10, GL-TES-SA-DME-E18-2, GL-TES-SA-DMP-E18-2, TL1-0ID-DMA and TL1-04D-DMA performed particularly well.

Non-cationic lipids particularly suitable for inclusion in such lipid nanoparticles include DOPE, DLoPE, DMPE, DLPE. DOPC, DEPE, DSPC and DPPC.

PEG-modified lipids particularly suitable for inclusion in such lipid nanoparticles include DMG-PEG2K and DSPE-PEG2K-COOH.

Cholesterol analogues particularly suitable for inclusion in such lipid nanoparticles include s-sitosterol and stigmastanol.

Exemplary lipid nanoparticles include one of GL-TES-SA-DME-E18-2, TL1-01 D-DMA, SY-3-E14-DMAPr, TL1-10D-DMA, GL-TES-SA-DMP-E18-2, HEP-E4-E10, HEP-E3-E10 and TL1-04D-DMA as a cationic lipid component. DOPE as a non-cationic lipid component, cholesterol as a helper lipid component, and DMG-PEG2K as a PEG-modified lipid component.

Table A below provides examples of lipid nanoparticles of the present invention. In one embodiment, the lipid nanoparticle of the present invention is any one of the lipid nanoparticles in Table A. In particular embodiments, the total lipid:mRNA ratio in the lipid nanoparticles of Table A is about 19:1 (mg:mg) or less. In particular embodiments, the total lipid:mRNA is between 11:1 and 19:1.

TABLE A
Exemplary lipid nanoparticles of the present invention

| | Molar | Molar | | |
|---|---|---|---|---|
| | ratio of | ratio of | Molar | Molar |
| | DMG-PEG2K | SY-3-E14- | ratio of | ratio of |
| LNP number | (%) | DMAPr (%) | cholesterol (%) | DOPE (%) |
| 1 | 5 | 50 | 35 | 10 |
| 2 | 5 | 50 | 32.5 | 12.5 |
| 3 | 5 | 50 | 30 | 15 |
| 4 | 5 | 50 | 27.5 | 17.5 |
| 5 | 5 | 55 | 30 | 10 |
| 6 | 5 | 55 | 27.5 | 12.5 |
| 7 | 5 | 55 | 25 | 15 |
| 8 | 5 | 55 | 22.5 | 17.5 |
| 9 | 5 | 60 | 25 | 10 |
| 10 | 5 | 60 | 22.5 | 12.5 |
| 11 | 5 | 60 | 20 | 15 |

Table B below provides a further example of a lipid nanoparticle of the present invention. In one embodiment, the total lipid:mRNA ratio in the lipid nanoparticle of Table B is about 19:1 (mg:mg) or less. In particular embodiments, the total lipid:mRNA is between 11:1 and 19:1.

TABLE B
Exemplary lipid nanoparticles of the present invention

| | Molar | Molar | Molar | |
|---|---|---|---|---|
| | ratio of | ratio of | ratio of | Molar |
| | DMG-PEG2K | SY-3-E14- | β-Sitosterol | ratio of |
| LNP number | (%) | DMAPr (%) | (%) | DOPE (%) |
| 14 | 5 | 50 | 30 | 15 |

Table C below provides a further example of a lipid nanoparticle of the present invention. In one embodiment, the total lipid:mRNA ratio in the lipid nanoparticle of Table C is about 19:1 (mg:mg) or less. In particular embodiments, the total lipid:mRNA is between 11:1 and 19:1.

TABLE C
Exemplary lipid nanoparticles of the present invention

| | Molar | Molar | Molar | |
|---|---|---|---|---|
| | ratio of | ratio of | ratio of | Molar |
| | DMG-PEG2K | SY-3-E14- | Stigmastanol | ratio of |
| LNP number | (%) | DMAPr (%) | (%) | DOPE (%) |
| 16 | 5 | 50 | 30 | 15 |

Table D below provides a further example of a lipid nanoparticle of the present invention. In one embodiment, the total lipid:mRNA ratio in the lipid nanoparticle of Table D is about 19:1 (mg:mg) or less. In particular embodiments, the total lipid:mRNA is between 11:1 and 19:1.

TABLE D
Exemplary lipid nanoparticles of the present invention

| | Molar ratio | Molar ratio of | | Molar ratio |
|---|---|---|---|---|
| LNP | of DMG- | SY-3-E14- | Molar ratio of | of DPPC |
| number | PEG2K (%) | DMAPr (%) | cholesterol (%) | (%) |
| 18 | 5 | 50 | 30 | 15 |

Table E below provides a further example of a lipid nanoparticle of the present invention. In one embodiment, the total lipid:mRNA ratio in the lipid nanoparticle of Table E is about 19:1 (mg:mg) or less. In particular embodiments, the total lipid:mRNA is between 11:1 and 19:1.

TABLE E
Exemplary lipid nanoparticles of the present invention

| | Molar ratio | Molar ratio of | Molar ratio of | Molar ratio |
|---|---|---|---|---|
| LNP | of DMG- | SY-3-E14- | β-Sitosterol | of DPPC |
| number | PEG2K (%) | DMAPr (%) | (%) | (%) |
| 19 | 5 | 50 | 30 | 15 |

Table F below provides a further example of a lipid nanoparticle of the present invention. In one embodiment, the total lipid:mRNA ratio in the lipid nanoparticle of Table F is about 19:1 (mg:mg) or less. In particular embodiments, the total lipid:mRNA is between 11:1 and 19:1.

TABLE F
Exemplary lipid nanoparticles of the present invention

| | Molar ratio | Molar ratio of | Molar ratio of | Molar ratio |
|---|---|---|---|---|
| LNP | of DMG- | SY-3-E14- | Stigmastanol | of DPPC |
| number | PEG2K (%) | DMAPr (%) | (%) | (%) |
| 20 | 5 | 50 | 30 | 15 |

Table G below provides a further example of a lipid nanoparticle of the present invention. In one embodiment, the total lipid:mRNA ratio in the lipid nanoparticle of Table G is about 19:1 (mg:mg) or less. In particular embodiments, the total lipid:mRNA is between 11:1 and 19:1.

TABLE G
Exemplary lipid nanoparticles of the present invention

| | Molar ratio | Molar ratio of | Molar ratio of | Molar ratio |
|---|---|---|---|---|
| LNP | of DMG- | SY-3-E14- | Cholesterol | of DOPE |
| number | PEG2K (%) | DMAPr (%) | (%) | (%) |
| 21 | 5 | 50 | 30 | 15 |

Table H below provides a further example of a lipid nanoparticle of the present invention. In one embodiment, the total lipid:mRNA ratio of the lipid nanoparticle of Table H is about 19:1 (mg:mg) or less. In particular embodiments, the total lipid:mRNA is between 11:1 and 19:1.

TABLE H
Exemplary lipid nanoparticles of the present invention

| | Molar ratio | Molar ratio of | Molar ratio of | |
|---|---|---|---|---|
| LNP | of DMG- | TL1-01D- | Cholesterol | Molar ratio |
| number | PEG2K (%) | DMA (%) | (%) | of DOPE (%) |
| 22 | 3 | 47 | 27.5 | 22.5 |

### Preparing Lipid Nanoparticles

Various processes can be used to prepare an mRNA-encapsulating lipid nanoparticle. Typically, the first step in preparing such a suspension is to provide a lipid solution. The lipid solution contains a mixture of the lipids that form the lipid nanoparticle. The lipid solution can be mixed with an mRNA solution, without first pre-forming the lipids into lipid nanoparticles, for encapsulation of mRNA (as described in U.S. patent application Ser. No. 14/790,562 entitled “Encapsulation of messenger RNA”, filed Jul. 2, 2015 and its provisional U.S. Patent Application No. 62/020,163, filed Jul. 2, 2014, and in International Patent Application WO 2016/004318, and in U.S. Patent Application No. 2016/0038432, both of which are hereby incorporated by reference in its entirety). Alternatively, a lipid solution is used to prepare lipid nanoparticles. The preformed lipid nanoparticles can then be mixed with an mRNA solution to encapsulate the mRNA in the preformed lipid nanoparticles, e.g., as described in International Patent Application WO 2018/089801, and in U.S. Patent Application No. 2018/0153822, both of which are hereby incorporated by reference in its entirety. These exemplary processes result in the effective encapsulation of mRNA in lipid nanoparticles. Typically, the processes can be optimized to achieve an encapsulation efficiency of at least about 90%, e.g., at least about 95%, at least about 96%, at least about 97%, at least about 98%, or at least about 99%.

As used herein, the term “encapsulation,” or grammatical equivalent, refers to the process of confining an mRNA molecule within a lipid nanoparticle. As used herein, this typically means that all, or substantially all, of the mRNA is encapsulated in the lipid nanoparticle.

The inventors have discovered that the encapsulation efficiency of the lipid nanoparticles of the invention remain relatively unaffected by nebulization, e.g., when a lipid nanoparticle in accordance with the invention is aerosolized by means of a vibrating mesh nebulizer. Accordingly, in some embodiments, the lipid nanoparticle of the present invention has an encapsulation efficiency before and after nebulization of at least about 90%. In some embodiments, the lipid nanoparticle of the present invention has an encapsulation efficiency before and after nebulization of at least about 95%. In some embodiments, the lipid nanoparticle of the present invention has an encapsulation efficiency before and after nebulization of at least about 96%. In some embodiments, the lipid nanoparticle of the present invention has an encapsulation efficiency before and after nebulization of at least about 97%. In some embodiments, the lipid nanoparticle of the present invention has an encapsulation efficiency before and after nebulization of at least about 98%. In some embodiments, the lipid nanoparticle of the present invention has an encapsulation efficiency before and after nebulization of at least about 99%.

The skilled artisan will appreciate that small loss in encapsulation efficiency upon nebulization of a lipid nanoparticle of the invention is acceptable, as long as the majority of the lipid nanoparticles (e.g., at least 80% of the lipid nanoparticles) in a composition of the invention effectively encapsulate the mRNA after they have been nebulized. Accordingly, in some embodiments, the encapsulation efficiency of the lipid nanoparticle of the present invention changes less than about 20% upon nebulization. In particular embodiments, the encapsulation efficiency of the lipid nanoparticle of the present invention changes less than about 15% upon nebulization. In a specific embodiment, the encapsulation efficiency of the lipid nanoparticle of the present invention changes less than about 10% upon nebulization. For example, in some embodiments, the encapsulation efficiency of the lipid nanoparticle of the present invention after nebulization is no more than about 20% lower than the encapsulation efficiency of the lipid nanoparticle before nebulization. In particular embodiments, the encapsulation efficiency of the lipid nanoparticle of the present invention after nebulization is no more than about 15% lower than the encapsulation efficiency of the lipid nanoparticle before nebulization. In a specific embodiment, the encapsulation efficiency of the lipid nanoparticle of the present invention after nebulization is no more than about 10% lower than the encapsulation efficiency of the lipid nanoparticle before nebulization. In another specific embodiment, the encapsulation efficiency of the lipid nanoparticle after nebulization is no more than about 5% lower than the encapsulation efficiency of the lipid nanoparticle before nebulization. In yet another specific embodiment, the encapsulation efficiency of the lipid nanoparticle after nebulization is no more than about 3% lower than the encapsulation efficiency of the lipid nanoparticle before nebulization. It is particularly desirable that the encapsulation efficiency of the lipid nanoparticle of the present invention after nebulization is about the same as the encapsulation efficiency of the lipid nanoparticle before nebulization.

Lipid:mRNA Ratio

A further advantage associated with the lipid nanoparticles of the invention is that they may require a smaller amount of total lipid for the effective encapsulation and delivery of mRNA in comparison to prior art mRNA-lipid nanoparticles commonly used for pulmonary delivery. For example, the lipid nanoparticle of the present invention may be prepared using a total lipid:mRNA ratio of less than 20:1 (mg:mg). In some embodiments, the lipid nanoparticle of the present invention is prepared using a total lipid:mRNA ratio between 11:1 and 19:1 (mg:mg). In some embodiments, the lipid nanoparticle of the present invention is prepared using a total lipid:mRNA ratio between 16:1 and 19:1 (mg:mg). In some embodiments, the lipid nanoparticle of the present invention is prepared using a total lipid:mRNA ratio of about 19:1 (mg:mg). In some embodiments, the lipid nanoparticle of the present invention is prepared using a total lipid:mRNA ratio of about 18:1 (mg:mg). In some embodiments, the lipid nanoparticle of the present invention is prepared using a total lipid:mRNA ratio of about 17:1 (mg:mg). In some embodiments, the lipid nanoparticle of the present invention is prepared using a total lipid:mRNA ratio of about 16:1 (mg:mg).

In some embodiments, the lipid nanoparticle of the present invention is prepared using a total lipid:mRNA ratio between 11:1 and 15:1 In some embodiments, the lipid nanoparticle of the present invention is prepared using a total lipid:mRNA ratio of about 15:1 (mg:mg). In some embodiments, the lipid nanoparticle of the present invention is prepared using a total lipid:mRNA ratio of about 14:1 (mg:mg). In some embodiments, the lipid nanoparticle of the present invention is prepared using a total lipid:mRNA ratio of about 13:1 (mg:mg). In some embodiments, the lipid nanoparticle of the present invention is prepared using a total lipid:mRNA ratio of about 12:1 (mg:mg). In some embodiments, the lipid nanoparticle of the present invention is prepared using a total lipid:mRNA ratio of about 11:1 (mg:mg).

Lipid:mRNA Ratio

In some embodiments, the lipid nanoparticle of the present invention has an N/P ratio of between 1 and 6. In particular embodiments, the lipid nanoparticle of the present invention has an N/P ratio of about 4. In some embodiments, the lipid nanoparticle of the present invention has an N/P ratio of less than 4. In some embodiments, the lipid nanoparticle of the present invention has an N/P ratio of about 3. In some embodiments, the lipid nanoparticle of the present invention has an N/P ratio of about 2.

### Lipid Nanoparticle Size

The processes for preparing a lipid nanoparticle of the invention referred to above yield compositions with a well-defined particle size. In some embodiments, the lipid nanoparticle of the present invention has a size less than about 150 nm. In specific embodiments, the lipid nanoparticle of the present invention has a size less than about 100 nm. In particular embodiments, the lipid nanoparticle of the present invention has a size of 60-150 nm, e.g., 60-125 nm, or 60-100 nm. Lipid nanoparticles within these size ranges have been used successfully to delivery mRNA to the lungs of a subject via nebulization.

In some embodiments, the lipid nanoparticle has a size of less than about 200 nm. In some embodiments, the lipid nanoparticle has a size of less than about 150 nm. In some embodiments, the lipid nanoparticle has a size of less than about 120 nm. In some embodiments, the lipid nanoparticle has a size of less than about 110 nm. In some embodiments, the lipid nanoparticle has a size of less than about 100 nm. In some embodiments, the lipid nanoparticle has a size of less than about 80 nm. In some embodiments, the lipid nanoparticle has a size of less than about 60 nm.

The size of a lipid nanoparticle of the invention may be determined by quasi-electric light scattering (QELS) as described in Bloomfield, Ann. Rev. Biophys. Bioeng., 10:421-150 (1981), incorporated herein by reference. For example, a Malvern Zetasizer can be used to measure the particle size in a lipid nanoparticle composition of the invention.

Messenger RNA (mRNA)

A lipid nanoparticle of the present invention can encapsulate any mRNA. mRNA is typically thought of as the type of RNA that carries information from DNA to the ribosome. Typically, in eukaryotic organisms, mRNA processing comprises the addition of a “cap” on the 5′ end, and a “tail” on the 3′ end. A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. The additional of a tail is typically a polyadenylation event whereby a polyadenylyl moiety is added to the 3′ end of the mRNA molecule. The presence of this “tail” serves to protect the mRNA from exonuclease degradation. mRNA is translated by the ribosomes into a series of amino acids that make up a protein.

mRNA Encoding a Therapeutic Protein

In certain embodiments, the mRNA encapsulated in a lipid nanoparticle of the invention encodes a therapeutic protein. The term “therapeutic protein” as used herein may refer to a protein, polypeptide or peptide. In a typical embodiment, a therapeutic protein is an enzyme, a membrane protein, an antibody or an antigen.

In some embodiments, the mRNA encapsulated in a lipid nanoparticle of the invention encodes for cystic fibrosis transmembrane conductance regulator (CFTR), ATP-binding cassette sub-family A member 3 protein, dynein axonemal intermediate chain 1 (DNAI1) protein, dynein axonemal heavy chain 5 (DNAH5) protein, alpha-1-antitrypsin protein, forkhead box P3 (FOXP3) protein, or a surfactant protein, e.g., surfactant A protein, surfactant B protein, surfactant C protein, and surfactant D protein.

In certain embodiments, the mRNA encapsulated in a lipid nanoparticle of the invention encodes cystic fibrosis transmembrane conductance regulator (CFTR) protein. In certain embodiments, the mRNA encapsulated in a lipid nanoparticle of the invention encodes a ATP-binding cassette sub-family A member 3 protein. In certain embodiments, the mRNA encapsulated in a lipid nanoparticle of the invention encodes dynein axonemal intermediate chain 1 (DNAI1) protein. In certain embodiments, the mRNA encapsulated in a lipid nanoparticle of the invention encodes dynein axonemal heavy chain 5 (DNAH5) protein. In certain embodiments, the mRNA encapsulated in a lipid nanoparticle of the invention encodes alpha-1-antitrypsin protein. In certain embodiments, the mRNA encapsulated in a lipid nanoparticle of the invention encodes forkhead box P3 (FOXP3) protein. In certain embodiments, the mRNA encapsulated in a lipid nanoparticle of the invention encodes a surfactant protein, e.g., more of surfactant A protein, surfactant B protein, surfactant C protein, and surfactant D protein.

In certain embodiments, the mRNA encapsulated in a lipid nanoparticle of the invention encodes an antigen. In certain embodiments, the mRNA encapsulated in a lipid nanoparticle of the invention encodes an antigen associated with a cancer of a subject or identified from a cancer cell of a subject. In certain embodiments, the mRNA encapsulated in a lipid nanoparticle of the invention encodes an antigen determined from a subject's own cancer cell (e.g., a tumor neoantigen), i.e., to provide a personalized cancer vaccine.

In certain embodiments, the mRNA encapsulated in a lipid nanoparticle of the invention encodes an antibody. In certain embodiments, the antibody can be a bi-specific antibody. In certain embodiments, the antibody can be part of a fusion protein. In certain embodiments, the codon optimized mRNA encapsulated in such lipid nanoparticle encodes for an antibody to OX40. In certain embodiments, the codon optimized mRNA encapsulated in such lipid nanoparticle encodes for an antibody to VEGF. In certain embodiments, the mRNA encapsulated in a lipid nanoparticle of the invention encodes an antibody to tissue necrosis factor alpha. In certain embodiments, the mRNA encapsulated in a lipid nanoparticle of the invention encodes an antibody to CD3. In certain embodiments, the mRNA encapsulated in a lipid nanoparticle of the invention encodes an antibody to CD19.

In certain embodiments, the mRNA encapsulated in a lipid nanoparticle of the invention encodes an immunomodulator. In certain embodiments, the mRNA encapsulated in a lipid nanoparticle of the invention encodes Interleukin 12. In certain embodiments, the mRNA encapsulated in a lipid nanoparticle of the invention encodes Interleukin 23. In certain embodiments, the mRNA encapsulated in a lipid nanoparticle of the invention encodes Interleukin 36 gamma. In certain embodiments, the mRNA encapsulated in a lipid nanoparticle of the invention encodes a constitutively active variant of one or more stimulator of interferon genes (STING) proteins.

In certain embodiments, the mRNA encapsulated in a lipid nanoparticle of the invention encodes an endonuclease. In certain embodiments, the mRNA encapsulated in a lipid nanoparticle of the invention encodes an RNA-guided DNA endonuclease protein, such as Cas 9 protein. In certain embodiments, the mRNA encapsulated in a lipid nanoparticle of the invention encodes meganuclease protein. In certain embodiments, the mRNA encapsulated in a lipid nanoparticle of the invention encodes a transcription activator-like effector nuclease protein. In certain embodiments, the mRNA encapsulated in a lipid nanoparticle of the invention encodes a zinc finger nuclease protein.

Typically, e mRNA encapsulated in a lipid nanoparticle of the invention comprises a poly-A tail. In some embodiments, the mRNA comprises a poly-A tail of at least 70 residues in length. In some embodiments, the mRNA comprises a poly-A tail of at least 100 residues in length. In some embodiments, the mRNA comprises a poly-A tail of at least 120 residues in length. In some embodiments, the mRNA comprises a poly-A tail of at least 150 residues in length. In some embodiments, the mRNA comprises a poly-A tail of at least 200 residues in length. In some embodiments, the mRNA comprises a poly-A tail of at least 250 residues in length.

mRNA synthesis

mRNAs may be synthesized according to any of a variety of known methods. Various methods are described in published U.S. Patent Application No. 2018/0258423, and can be used to practice the present invention, all of which are incorporated herein by reference. For example, mRNAs for use with the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. In some embodiments, mRNA may be further purified for use with the present invention. In some embodiments, in vitro synthesized mRNA may be purified before formulation and encapsulation to remove undesirable impurities including various enzymes and other reagents used during mRNA synthesis.

Various methods may be used to purify mRNA for use with the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art.

In particular embodiments, the mRNA is purified using Tangential Flow Filtration (TFF). Suitable purification methods include those described in published U.S. Application No. 2016/0040154, published U.S. Patent Application No. 2015/0376220, published U.S. Patent Application No. 2018/0251755, published U.S. Patent Application No. 2018/0251754, U.S. Provisional Patent Application No. 62/757,612 filed on Nov. 8, 2018, and U.S. Provisional Patent Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing. In some embodiments, the mRNA is purified either before or after or both before and after capping and tailing, by centrifugation. In some embodiments, the mRNA is purified either before or after or both before and after capping and tailing, by filtration. In some embodiments, the mRNA is purified either before or after or both before and after capping and tailing, by TFF. In some embodiments, the mRNA is purified either before or after or both before and after capping and tailing by chromatography.

Various naturally-occurring or modified nucleosides may be used to produce an mRNA for use with the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine): chemically modified bases: biologically modified bases (e.g., methylated bases); intercalated bases: modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO201012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

The lipid nanoparticles of the invention may encapsulate mRNAs of a variety of lengths. In some embodiments, the lipid nanoparticles of the present invention may encapsulate in vitro synthesized mRNA of or greater than about 0.5 kb, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, 20 kb, 30 kb, 40 kb, or 50 kb in length. In some embodiments, the lipid nanoparticles of the present invention may encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, about 8-15 kb, or about 8-50 kb in length.

Codon Optimized mRNA

In some embodiments, the lipid nanoparticle of the present invention is for delivering codon optimized mRNA encoding a therapeutic protein to a subject for the treatment of a disease. A suitable codon optimized mRNA encodes any full length, fragment or portion of a protein which can be substituted for naturally-occurring protein activity and/or reduce the intensity, severity, and/or frequency of one or more symptoms associated with the disease.

### Generation of Optimized Nucleotide Sequences

The present invention provides lipid nanoparticles that may encapsulate mRNAs that comprise optimized nucleotide sequence encoding a therapeutic protein. These mRNAs are modified relative to their naturally occurring counterparts to (a) improve the yield of full-length mRNAs during in vitro synthesis, and (b) to maximize expression of the encoded polypeptide after delivery of the mRNA to a target cell in vivo. Sequence motifs that favour rapid degradation of the mRNA in the target cell have also been removed.

An exemplary process for generating optimized nucleotide sequences for mRNA encapsulated by the lipid nanoparticles of the present invention first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded polypeptide.

### Codon Optimization

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome.

A codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

Codon usage tables are stored in publicly available databases, such as the Codon Usage Database (Nakamura et al. (2000) Nucleic Acids Research 28(1), 292: available online at https://www.kazusa.or.jp/codon/), and the High-performance Integrated Virtual Environment-Codon Usage Tables (HIVE-CUTs) database (Athey et al., (2017), BMC Bioinformatics 18(1), 391; available online at http://hive.biochemistry.gwu.edu/review/codon).

During the first step of codon optimization, codons are removed from a first codon usage table which reflects the frequency of each codon in a given organism (e.g., a mammal or human) if they are associated with a codon usage frequency which is less than a threshold frequency (e.g., 10%). The codon usage frequencies of the codons not removed in the first step are normalized to generate a normalized codon usage table. An optimized nucleotide sequence encoding an amino acid sequence of interest is generated by selecting a codon for each amino acid in the amino acid sequence based on the usage frequency of the one or more codons associated with a given amino acid in the normalized codon usage table. The probability of selecting a certain codon for a given amino acid is equal to the usage frequency associated with the codon associated with this amino acid in the normalized codon usage table.

The codon-optimized sequences of the mRNA encapsulated by the lipid nanoparticles of the present invention are generated by a computer-implemented method for generating an optimized nucleotide sequence. The method comprises: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. The threshold frequency can be in the range of 5% 30%, in particular 5%, 10%, 15%, 20%, 25%, or 30%. In the context of the present invention, the threshold frequency is typically 10%.

The step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon. “Distributed” in this context may be defined as taking the combined magnitude of the usage frequencies of removed codons associated with a certain amino acid and apportioning some of this combined frequency to each of the remaining codons encoding the certain amino acid.

The step of selecting a codon for each amino acid comprises: (a) identifying, in the normalized codon usage table, the one or more codons associated with a first amino acid of the amino acid sequence: (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

The step of generating an optimized nucleotide sequence by selecting a codon for each amino acid in the amino acid sequence (step (v) in the above method) is performed n times to generate a list of optimized nucleotide sequences.

### Motif Screen

A motif screen filter is applied to the list of optimized nucleotide sequences. Optimized nucleotide sequences encoding any known negative cis-regulatory elements and negative repeat elements are removed from the list to generate an updated list.

For each optimized nucleotide sequence in the list, it is also determined whether it contains a termination signal. Any nucleotide sequence that contains one or more termination signals is removed from the list generating an updated list. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTTT; and/or AAGCTT; and/or GAAGAGC; and/or TCTAGA. In a typical embodiment, the termination signal has the following nucleotide sequence: 5′-X₁AUCUX₂UX₃-3′, wherein X₁, X₂ and X₃ are independently selected from A, C. U or G. In a specific embodiment, the termination signal has one of the following nucleotide sequences: UAUCUGUU; and/or UUUUUU; and/or AAGCUU; and/or GAAGAGC; and/or UCUAGA.

### Guanine-Cytosine (GC) Content

The method further comprises determining a guanine-cytosine (GC) content of each of the optimized nucleotide sequences in the updated list of optimized nucleotide sequences. The GC content of a sequence is the percentage of bases in the nucleotide sequence that are guanine or cytosine. The list of optimized nucleotide sequences is further updated by removing any nucleotide sequence from the list, if its GC content falls outside a predetermined GC content range.

Determining a GC content of each of the optimized nucleotide sequences comprises, for each nucleotide sequence: determining a GC content of one or more additional portions of the nucleotide sequence, wherein the additional portions are non-overlapping with each other and with the first portion, and wherein updating the list of optimized sequences comprises: removing the nucleotide sequence if the GC content of any portion falls outside the predetermined GC content range, optionally wherein determining the GC content of the nucleotide sequence is halted when the GC content of any portion is determined to be outside the predetermined GC content range. In some embodiments, the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides. In the context of the present invention, the predetermined number of nucleotides is typically 30 nucleotides. The predetermined GC content range can be 15%-75%, or 40%-60%, or, 30%-70%. In the context of the present invention, the predetermined GC content range is typically 30%-70%.

A suitable GC content filter in the context of the invention may first analyze the first 30 nucleotides of the optimized nucleotide sequence, i.e., nucleotides 1 to 30 of the optimized nucleotide sequence. Analysis may comprise determining the number of nucleotides in the portion with are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list.

### Codon Adaptation Index (CAI)

The method further comprises determining a codon adaptation index of each of the optimized nucleotide sequences in the most recently updated list of optimized nucleotide sequences. The codon adaptation index of a sequence is a measure of codon usage bias and can be a value between 0 and 1. The most recently updated list of optimized nucleotide sequences is further updated by removing any nucleotide sequence if its codon adaptation index is less than or equal to a predetermined codon adaptation index threshold. The codon adaptation index threshold can 0.7, or 0.75, or 0.8, or 0.85, or 0.9. The inventors have found that optimized nucleotide sequences with a codon adaptation index equal to or greater than 0.8 deliver very high protein yield. Therefore in the context of the invention, the codon adaptation index threshold is typically 0.8.

A codon adaptation index may be calculated, for each optimized nucleotide sequence, in any way that would be apparent to a person skilled in the art, for example as described in “The codon adaptation index—a measure of directional synonymous codon usage bias, and its potential applications” (Sharp and Li, 1987. Nucleic Acids Research 15(3), p. 1281-1295); available online at https://www.ncbi.nlm.nih.gov/pmc/articles/PMC340524/.

Implementing a codon adaptation index calculation may include a method according to, or similar to, the following. For each amino acid in a sequence, a weight of each codon in a sequence may be represented by a parameter termed relative adaptiveness (wᵢ). Relative adaptiveness may be computed from a reference sequence set, as the ratio between the observed frequency of the codon fᵢ and the frequency of the most frequent synonymous codon fⱼ for that amino acid. The codon adaptation index of a sequence may then be calculated as the geometric mean of the weight associated to each codon over the length of the sequence (measured in codons). The reference sequence set used to calculate a codon adaptation index may be the same reference sequence set from which a codon usage table used with the codon optimization methods described herein is derived.

### Compositions

The invention provides compositions comprising the lipid nanoparticles of the invention. In a typical embodiment, such compositions are formulated for pulmonary delivery by nebulization. Formulation may include the addition of various excipients. These excipients may be useful in maintaining the encapsulation efficiency before and after nebulization of the lipid nanoparticle composition. Nebulization in the context of the present disclosure is commonly performed with a nebulizer comprising vibrating mesh technology (VMT).

In some embodiments, a composition in accordance with the invention comprises an mRNA encapsulated in the lipid nanoparticle of the invention, wherein the mRNA is present in the composition at a concentration ranging from about 0.5 mg/mL to about 1.0 mg/mL. In some embodiments, the mRNA is present at a concentration of at least 0.5 mg/mL. In some embodiments, the mRNA is present at a concentration of at least 0.6 mg/mL. In some embodiments, the mRNA is present at a concentration of at least 0.7 mg/mL. In some embodiments, the mRNA is present at a concentration of at least 0.8 mg/mL. In some embodiments, the mRNA is present at a concentration of at least 0.9 mg/mL. In some embodiments, the mRNA is present at a concentration of at least 1.0 mg/mL. In a typical embodiment, the mRNA is present at a concentration of about 0.6 mg/mL to about 0.8 mg/mL.

The compositions of the invention may be formulated with one or more carrier, stabilizing reagent or other excipients. Such compositions may be pharmaceutical compositions, and as such they may include one more or more pharmaceutically acceptable excipients. The one or more pharmaceutically acceptable excipients may be selected from a buffer, a sugar, a salt, a surfactant or combinations thereof.

In specific embodiments, the composition of the present invention comprises a buffer. In certain embodiments, the composition of the present invention comprises a salt, e.g., sodium chloride. In some embodiments, the composition of the present invention comprises a sugar, e.g., a disaccharide (such as sucrose or trehalose) at a suitable concentration, e.g., about 4% w/v, about 6% w/v, about 8% w/v, or about 10% w/v.

In some embodiments, a composition of the present invention is stable at room temperature (e.g., for at least 12 hours or 24 hours), or at −20° C. (e.g., for at least 6 months or a year). In particular embodiments, a composition of the present invention is provided in lyophilized form and is reconstituted into an aqueous solution (e.g., water for injection) prior to nebulization. Such compositions typically comprise a lyoprotectant. A suitable lyoprotectant for use with the lipid nanoparticles of the invention may be a disaccharide (such as sucrose or trehalose).

### Pharmaceutically Acceptable Excipients

In some embodiments, the pharmaceutical composition is formulated with a diluent. In some embodiments, the diluent is selected from a group consisting of ethylene glycol, glycerol, propylene glycol, sucrose, trehalose, or combinations thereof. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

In some embodiments, the LNPs are suspended in an aqueous solution comprising a disaccharide. Suitable disaccharide for use with the invention include trehalose and sucrose. For example, in some embodiments, the LNPs are suspended in an aqueous solution comprising trehalose, e.g., 10% (w/v) trehalose in water. In other embodiments, LNPs are suspended in an aqueous solution comprising sucrose, e.g., 10% (w/v) sucrose in water.

In some embodiments, the aqueous solution further comprises a buffer, a salt, a surfactant or combinations thereof.

In some embodiments, the salt is selected from the group consisting of NaCl, KCl, and CaCl₂. Accordingly, in some embodiments, the salt is NaCl. In some embodiments, the salt is KCl. In some embodiments, the salt is CaCl₂.

In some embodiments, the buffer is selected from the group consisting of a phosphate buffer, a citrate buffer, an imidazole buffer, a histidine buffer, and a Good's buffer. Accordingly, in some embodiments, the buffer is a phosphate buffer. In some embodiments, the buffer is a citrate buffer. In some embodiments, the buffer is an imidazole buffer. In some embodiments, the buffer is a histidine buffer. In some embodiments, the buffer is a Good's buffer. In some embodiments, the Good's buffer is a Tris buffer or HEPES buffer.

In particular embodiments, the buffer is a phosphate buffer (e.g., a citrate-phosphate buffer), a Tris buffer, or an imidazole buffer.

In particular embodiments, a composition in accordance with the present invention comprises a buffer and a salt (typically in addition to a suitable diluent such as a disaccharide or optionally a propylene glycol), e.g., in order to enhance the stability of the composition during storage. In some embodiments, the total concentration of the buffer and the salt is selected from about 40 mM Tris buffer and about 75-125 mM NaCl, about 50 mM Tris buffer and about 50 mM-100 mM NaCl, about 100 mM Tris buffer and about 100 mM-200 mM NaCl, about 40 mM imidazole and about 100 mM-125 mM NaCl, and about 50 mM imidazole and 75 mM-100 mM NaCl.

### Disaccharides

Disaccharides such as trehalose and sucrose are excipients that can maintain stability of lipid nanoparticles of the invention during nebulization, and in some embodiments, also during lyophilization.

A sugar:lipid ratio of about 7 to about 9 is typically sufficient to maintain stability of the lipid nanoparticles. In some embodiments, even lower ratios may be acceptable. For examples, as can be seen from the examples, a disaccharide concentration of less than 10%, e.g., about 4% to about 8%, is effective in maintain size and encapsulation efficiency of the lipid nanoparticles of the invention post-nebulization. Such lipid nanoparticles formulations can also be lyophilized without significant increase in size or loss in encapsulation efficiency after lyophilization and reconstitution.

Sucrose in particular has been found to be an effective excipient. In some embodiments, sucrose may be used as the sole excipient, e.g., at a concentration of less than 10%. e.g., between about 4% and about 8%. e.g., about 8%. In other embodiments, sucrose may be combined with a buffer (e.g., a phosphate buffer) and a salt (e.g., NaCl).

### Exemplary Compositions

In some embodiments, a composition comprising a lipid nanoparticle of the invention comprises an mRNA (typically at a concentration of 0.4-0.8 mg/ml) encapsulated in the lipid nanoparticle, a disaccharide such as trehalose or sucrose at a concentration (w/v) of about 3-10%, and optionally TPGS at a concentration (w/v) of about 0.1-1%. In a particular embodiment, a composition of the invention comprises an mRNA at a concentration of about 0.6 mg/ml encapsulated in the lipid nanoparticle, trehalose at a concentration (w/v) of about 8%, and TPGS at a concentration (w/v) of about 0.5%. In another particular embodiment, a composition of the invention comprises an mRNA at a concentration of about 0.6 mg/ml encapsulated in the lipid nanoparticle, and sucrose at a concentration (w/v) of about 8%.

In some embodiments, a composition comprising a lipid nanoparticle of the invention comprises an mRNA encapsulated in the lipid nanoparticle, a disaccharide such as trehalose or sucrose at a concentration (w/v) of about 3-8%, a buffer (e.g., a phosphate buffer), and a salt (e.g., sodium chloride). In some embodiments, the composition comprises the mRNA at a concentration of 0.4-0.8 mg/ml encapsulated in the lipid nanoparticle, trehalose or sucrose at a concentration (w/v) of about 4%-6%, a phosphate buffer at 1 mM-10 mM (pH 5-5.5) and sodium chloride at a concentration of at least 75 mM (e.g., about 75 mM to 200 mM). In a particular embodiment, the composition comprises the mRNA at a concentration of about 0.4 mg/ml encapsulated in the lipid nanoparticle, sucrose at a concentration (w/v) of about 4%, a phosphate buffer at about 2.5 mM (pH 5.5) and sodium chloride at a concentration of about 150 mM). In another particular embodiment, the composition comprises the mRNA at a concentration of about 0.4 mg/ml encapsulated in the lipid nanoparticle, trehalose at a concentration (w/v) of about 4%, a phosphate buffer at about 10 mM (pH 5) and sodium chloride at a concentration of about 150 mM).

In specific embodiments, a composition comprising an mRNA-encapsulating lipid nanoparticle of the invention comprises the mRNA at a concentration of about 0.6 mg/mL, sucrose at a concentration of about 8% (w/v), and the molar ratios of the lipid components of the lipid nanoparticle are: a. about 50% SY-3-E14-DMAPr; b. about 15% DOPE; c. about 5% DMG-PEG2K; and d. about 30% cholesterol.

In specific embodiments, a composition comprising an mRNA-encapsulating lipid nanoparticle of the invention comprises the mRNA at a concentration of about 0.4 mg/ml, the disaccharide is sucrose at a concentration of about 4% (w/v), a phosphate buffer at a concentration of about 2.5 mM (pH 5.5), sodium chloride at a concentration of about 150 mM, and the molar ratios of the lipid components of the lipid nanoparticle are: a. about 47% TL1-01D-DMA: b. about 22.5% DOPE: c. about 3% DMG-PEG2K; and d. about 27.5% cholesterol.

### Dry Powder Formulations

The invention also provides dry powder formulations comprising a plurality of spray-dried particles comprising the lipid nanoparticles of the invention. Typically, a dry powder formulation suitable for use with the invention includes one or more polymers. Additionally, it may include one or more other excipients, e.g., one or more sugars or sugar alcohols and/or one or more surfactants. In a typical embodiment of the invention, a dry powder formulation comprises lipid nanoparticles of the invention, one or more polymers (e.g., polymethacrylate-based polymer such as Eudragit EPO), one or more sugars or sugar alcohols or combinations thereof (e.g., mannitol, or mannitol and lactose or mannitol and trehalose), and optionally one or more surfactants (e.g., a poloxamer such as poloxamer 407). Providing the lipid nanoparticles of the invention as a dry powder formulation is advantageous because such formulations are stable. In particular embodiments, the spray-dried particles are administered via nebulization.

### Sugars or Sugar Alcohols

Exemplary sugars or sugar alcohols suitable for use with a dry powder formulation in accordance with the invention are monosaccharides, disaccharides and polysaccharides, selected from a group consisting of glucose, fructose, galactose, mannose, sorbose, lactose, sucrose, cellobiose, trehalose, raffinose, starch, dextran, maltodextrin, cyclodextrins, inulin, xylitol, sorbitol, lactitol, and mannitol.

In some embodiments, a suitable sugar or sugar alcohol is lactose and/or mannitol. In some embodiments, a suitable sugar is mannitol. In some embodiments, the mannitol is added at a concentration of about 1-10%. In some embodiments, the mannitol is added at a concentration of about 2-10%. In some embodiments, the mannitol is added at a concentration of about 3-10%. In some embodiments, the mannitol is added at a concentration of about 4-10%. In some embodiments, the mannitol is added at a concentration of about 5-10%.

In some embodiments, a suitable sugar is trehalose. In some embodiments, both mannitol and trehalose are added.

### Surfactants

In some embodiments, a dry formulation in accordance with the invention further comprises one or more surfactants. Surfactants increase the surface tension of a composition. In some embodiments, the surfactants used in spray-drying mRNA lipid compositions are selected from a group consisting of CHAPS (3-[(3-Cholamidopropyl)dimethylammonio]-1-propanesulfonate), phospholipids, phosphatidylserine, phosphatidylethanolamine, phosphatidylcholine, sphingomyelins, octaethylene glycol monododecyl ether, pentaethylene glycol monododecyl ether, Triton X-100, Cocamide monoethanolamine, Cocamide diethanolamine, Glycerol monostearate, Glycerol monolaurate, Sorbitan moonolaureate, Sorbitan monostearate, Tween 20, Tween 40, Tween 60, Tween 80, Alkyl polyglucosides, and poloxamers.

In a particular embodiment, the surfactant is a poloxamer (e.g., poloxamer 407).

### Nebulization

Compositions comprising the lipid nanoparticles of the invention are typically administered by pulmonary delivery, in particular by nebulization. Nebulization results in an aerosolized composition which can be inhaled. Upon inhalation, the lipid nanoparticles are distributed throughout the nose, airways and the lungs and taken up by the epithelial cells of these tissues. As a consequence, the mRNA encapsulated in the lipid nanoparticles is delivered into the cells and expressed, e.g., in the nasal cavity, trachea, bronchi, bronchioles, and/or other pulmonary system-related cells or tissues.

Additional teaching of pulmonary delivery and nebulization are described, e.g., in WO2018089790A1 and WO2018213476A1, each of which is incorporated by reference in its entirety.

Inhaled aerosol droplets of a particle size of less than 8 μm (e.g., 1-5 μm) can penetrate into the narrow branches of the lower airways. Aerosol droplets with a larger diameter are typically absorbed by the epithelial cells lining the oral cavity and upper airway, and are unlikely to reach the lower airway epithelium and the deep alveolar lung tissue. Accordingly, in particular in the context of pulmonary delivery to the lung epithelium, methods that comprise administering an mRNA encapsulated in lipid nanoparticles of the invention as an aerosol may include steps of generating droplets of a particle size of less than 8 μm (e.g., 1-5 μm), typically by nebulization of a composition of the invention, e.g., by using a nebulizer that is suitable for use with the compositions of the invention.

In a specific embodiment, a composition of the invention is nebulized to generate nebulized particles for inhalation by the subject. In particular embodiments, the lipid nanoparticle of the present invention is capable of being nebulized at a nebulization output rate of greater than about 12 ml/h. In particular embodiments, the lipid nanoparticle of the present invention is capable of being nebulized at a nebulization output rate of greater than about 15 ml/h. In other particular embodiments, the lipid nanoparticle of the present invention is capable of being nebulized at a nebulization output rate of greater than about 30 ml/h. In some embodiments, the lipid nanoparticle of the present invention is capable of being nebulized at a nebulization output rate of 12-50 ml/h. In some embodiments, the lipid nanoparticle of the present invention is capable of being nebulized at a nebulization output rate of 12-40 ml/h. In some embodiments, the lipid nanoparticle of the present invention is capable of being nebulized at a nebulization output rate of 15-50 ml/h. In some embodiments, the lipid nanoparticle of the present invention is capable of being nebulized at a nebulization output rate of 15-40 m/h. In a typical embodiment, the lipid nanoparticle of the present invention is capable of being nebulized at a nebulization output rate of between 12 ml/h and about 30 ml/h, e.g., between 15 ml/h and about 30 ml/h. For example, a lipid nanoparticle of the present invention is capable of being nebulized with a nebulization output rate of about 12 m/h or about 15 ml/h. In particular embodiments, a lipid nanoparticle of the present invention can be nebulized at a nebulization output rate of about 30 ml/h.

Typically, in the context of the present invention, a lipid nanoparticle that is capable of being nebulized at a higher nebulization output rate retains the capability of effectively encapsulating the mRNA after nebulization, such that the majority of the lipid nanoparticles (e.g., at least 80%, e.g., at least 85%, particularly at least 90% of the lipid nanoparticles) in a composition of the invention encapsulate mRNA after they have been nebulized. Accordingly, in some embodiments, the encapsulation efficiency of the lipid nanoparticle of the present invention changes less than about 20% upon nebulization. In particular embodiments, the encapsulation efficiency of the lipid nanoparticle of the present invention changes less than about 15% upon nebulization. In a specific embodiment, the encapsulation efficiency of the lipid nanoparticle of the present invention changes less than about 10% upon nebulization. For example, in some embodiments, the encapsulation efficiency of the lipid nanoparticle of the present invention after nebulization is no more than about 20% lower than the encapsulation efficiency of the lipid nanoparticle before nebulization. In particular embodiments, the encapsulation efficiency of the lipid nanoparticle of the present invention after nebulization is no more than about 15% lower than the encapsulation efficiency of the lipid nanoparticle before nebulization. In a specific embodiment, the encapsulation efficiency of the lipid nanoparticle of the present invention after nebulization is no more than about 10% lower than the encapsulation efficiency of the lipid nanoparticle before nebulization.

Nebulized particles for inhalation by a subject typically have an average size less than 8 μm. In some embodiments, the nebulized particles for inhalation by a subject have an average size between approximately 1-8 μm. In particular embodiments, the nebulized particles for inhalation by a subject have an average size between approximately 1-5 μm. In specific embodiments, the mean particle size of the nebulized composition of the invention is between about 4 μm and 6 μm, e.g., about 4 μm, about 4.5 μm, about 5 μm, about 5.5 μm, or about 6 μm.

Particle size in an aerosol is commonly described in reference to the Mass Median Aerodynamic Diameter (MMAD). MMAD, together with the geometric standard deviation (GSD), describes the particle size distribution of any aerosol statistically, based on the weight and size of the particles. Means of calculating the MMAD of an aerosol are well known in the art. For example, the MMAD output of a nebulizer using a composition of the invention can be determined using a Next Generation Impactor. Another parameter to describe particle size in an aerosol is the Volume Median Diameter (VMD). VMD also describes the particle size distribution of an aerosol based on the volume of the particles. Means of calculating the VMD of an aerosol are well known in the art. A specific method used for determining the VMD is laser diffraction, which is used herein to measure the VMD of a composition of the invention (see, e.g., Clark, 1995, Int J Pharm. 115:69-78).

Accordingly, in some embodiments, nebulization in accordance with the invention is performed to generate a Fine Particle Fraction (FPF), which is defined as the proportion of particles in an aerosol which have an MMAD or a VMD smaller than a specified value. In one specific embodiment, the FPF of a nebulized composition of the invention with a particle size <5 μm is at least about 30%, more typically at least about 40%, e.g., at least about 50%, more typically at least about 60%. In another specific embodiment, nebulization is performed in such a manner that the mean respirable emitted dose (i.e., the percentage of FPF with a particle size <5 μm; e.g., as determined by next generation impactor with 15 L/min extraction) is at least about 30% of the emitted dose, e.g., at least about 31%, at least about 32%, at least about 33%, at least about 34%, or at least about 35% the emitted dose. In yet another specific embodiment, nebulization is performed in such a manner that the mean respirable delivered dose (i.e., the percentage of FPF with a particle size <5 μm; e.g., as determined by next generation impactor with 15 L/min extraction) is at least about 15% of the emitted dose, e.g., at least 16% or 16.5% of the emitted dose.

In some embodiments, nebulization is performed with a nebulizer. One type of nebulizer is a jet nebulizer, which comprises tubing connected to a compressor, which causes compressed air or oxygen to flow at a high velocity through a liquid medicine to turn it into an aerosol, which is then inhaled by the subject. Another type of nebulizer is the ultrasonic wave nebulizer, which comprises an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Exemplary ultrasonic wave nebulizers are the Omron NE-U17 and the Beurer Nebulizer IH30. A third type of nebulizer comprises vibrating mesh technology (VMT). A VMT nebulizer typically comprises a mesh/membrane with 1000-7000 holes that vibrates at the top of a liquid reservoir and thereby pressures out a mist of very fine aerosol droplets through the holes in the mesh/membrane. Exemplary VMT nebulizers include eFlow (PARI Medical Ltd.), i-Neb (Respironics Respiratory Drug Delivery Ltd), Nebulizer IH50 (Beurer Ltd.), AeroNeb Go (Aerogen Ltd.), InnoSpire Go (Respironics Respiratory Drug Delivery Ltd), Mesh Nebulizer (Shenzhen Homed Medical Device Co, Ltd), Portable Nebulizer (Microbase Technology Corporation) and Airworks (Convexity Scientific LLC). In some embodiments, the mesh or membrane of the VMT nebulizer is made to vibrate by a piezoelectric element. In some embodiments, the mesh or membrane of the VMT nebulizer is made to vibrate by ultrasound.

VMT nebulizers have been found to be particularly suitable for practicing the invention because they do not affect the mRNA integrity of the mRNA encapsulated within LNPs of the invention, present in a composition. Typically, at least about 60%, e.g., at least about 65% or at least about 70%, of the mRNA in the compositions of the invention maintains its integrity after nebulization.

In some embodiments, nebulization is continuous during inhalation and exhalation. More typically, nebulization is breath-actuated. Suitable nebulizers for use with the invention have nebulization rate greater than 0.2 mL/min. In some embodiments, the nebulization rate is greater than 0.25 mL/min. In other embodiment, the nebulization rate is greater than 0.3 mL/min. In certain embodiments, the nebulization rate is greater than 0.45 mL/min. In a typical embodiment, the nebulization rate ranges between 0.2 mL/min and 0.5 mL/min.

### Therapeutic Use of Compositions

The invention provides methods of delivering lipid nanoparticles of the invention in vivo comprising administering the compositions of the invention via pulmonary delivery to a subject. In some embodiments, the subject is human. In certain embodiments, the pulmonary delivery can be by intranasal administration or inhalation. In certain embodiments, the composition is nebulized prior to inhalation.

The mRNA encapsulated in the lipid nanoparticles of the invention encodes a protein. In some embodiments, the mRNA is delivered to the lungs. In particular embodiments, the protein encoded by the mRNA is expressed in the lung. The protein can, for example, be a secreted protein, such as an antibody. In some embodiment, the protein can be a membrane protein, such as a viral surface antigen, a cell surface receptor, or a membrane channel (e.g., cystic fibrosis transmembrane conductance regulator (CFTR)). The protein expressed by the mRNA typically has therapeutic activity. For example, the expressed protein can be used to treat or prevent a disease or disorder. Accordingly, the lipid nanoparticles and compositions of the invention are for use in the treatment or prevention of a disease or disorder. Typically, such use comprises pulmonary administration of the lipid nanoparticles or compositions, e.g., via nebulization. In some embodiments, the lipid nanoparticles and compositions are for use in the manufacture of a medicament for the treatment or prevention of a disease or disorder. In a typical embodiment, such manufacture includes the formulation of the lipid nanoparticles in compositions, which are suitable for pulmonary administration, e.g., via nebulization. The invention also provides methods of treating or preventing a disease or disorder in a subject, the method comprising administering the composition of the invention via pulmonary delivery to the subject. In some embodiments, the pulmonary delivery is via nebulization.

The methods of the invention can be used to treat a variety of diseases and disorders in a subject, such as pulmonary diseases, e.g., chronic respiratory diseases; protein deficiencies, e.g., protein deficiencies affecting the lungs; neoplastic diseases, e.g., tumors; and infectious disease. In particular embodiments, the disease or disorder is a protein deficiency. In some embodiments, the subject is healthy, in which case treatment is for the prevention of a disease or disorder (e.g., by immunisation with an mRNA-encoded antigen to prevent an infectious disease). In other embodiments, the subject is suffering from a disease or disorder, in which case the treatment may be aimed at reducing or ameliorating one or more symptoms of the disease or disorder, and/or at addressing the underlying cause of the disease, e.g., by providing a deficient protein through delivery of an mRNA encoding the same, or by supplying an agent that targets the diseased tissue, such as an antibody that interferes with tumor growth.

In certain embodiments, the mRNA encodes a protein that is deficient in a subject. Examples of protein deficiencies that can be treated are cystic fibrosis, primary ciliary dyskinesia or surfactant deficiency. Expression of the mRNA in the lungs may partially or totally restore the level of the protein in the subject. For example, the methods of the invention result in a subject having protein levels that are comparable to a healthy subject. In certain embodiments, the methods of the invention result in a 20%, 30%, 40%, 50%, 60%, 70%, 80%, 90% or 100% in production of the protein.

In some embodiments, the invention provides methods of treating cystic fibrosis in a subject, the method comprising administering the compositions of the invention via pulmonary delivery to the subject. In some embodiments, the mRNA encodes CFTR. In some embodiments, the invention provides methods of treating primary ciliary dyskinesia in a subject, the method comprising administering the compositions of the invention via pulmonary delivery to the subject. In some embodiments, the mRNA encodes DNAI1. In some embodiments, the invention provides methods of treating a surfactant deficiency in a subject, the method comprising administering the compositions of the invention via pulmonary delivery to the subject. In some embodiments, the mRNA encodes a surfactant protein.

In some embodiments, the invention provides methods of treating a chronic respiratory disease in a subject, the method comprising administering the compositions of the invention via pulmonary delivery to the subject. Examples of chronic respiratory diseases that can be treated with the methods of the invention include chronic obstructive pulmonary disease (COPD), asthma, pulmonary arterial hypertension or idiopathic pulmonary fibrosis. In some embodiments, the mRNA encodes a protein for treating a symptom of a pulmonary disease or disorder. In certain embodiments, the mRNA encodes an antibody directed against a pro-inflammatory cytokine.

In some embodiments, the invention provides methods of treating or preventing a neoplastic disease, e.g., a tumor, in a subject, the method comprising administering the compositions of the invention via pulmonary delivery to the subject. In some embodiments, the tumor is a lung tumor or lung cancer, for example non-small cell lung cancer or small cell lung cancer. In certain embodiments, the mRNA encodes an antibody that targeting a protein expressed on the surface of cells making up the tumor. In other embodiments, the mRNA encodes an antigen derived from the tumor, e.g., a tumor neoantigen.

In some embodiments, the invention provides methods of treating or preventing an infectious disease in a subject, the method comprising administering the compositions of the invention via pulmonary delivery to the subject. In some embodiments, the infectious disease is caused by a virus. In some embodiments, the infectious disease is a pulmonary infectious disease or disorder. In some embodiments the mRNA encodes a soluble decoy receptor that binds a surface protein of the virus. In some embodiments the mRNA encodes an antibody directed to a surface protein of the virus. In some embodiments, the infectious disease is caused by a bacterium. In particular embodiments, the mRNA encodes an antibody directed to a surface protein of the bacterium. In some embodiments, the mRNA encodes an antigen derived from a causative agent of the infections disease (e.g., a surface protein derived from a virus or a bacterium which causes the infectious disease). For example, a lipid nanoparticle of the invention encapsulating an mRNA encoding the antigen, or a composition comprising the lipid nanoparticle may be used to immunize a subject to prevent the infectious disease in the subject.

### EXAMPLES

While certain lipid nanoparticles, compositions and methods of the present invention have been described with specificity in accordance with certain embodiments, the following examples serve only to illustrate the invention and are not intended to limit the same.

### Example 1. Preparation of Lipid Nanoparticles

The lipid nanoparticles in the Tables 1A through 1F were prepared to investigate the effect of lowering the molar ratio of non-cationic lipid on the nebulization properties of the lipid nanoparticle. As a control, lipid nanoparticles were also prepared with the non-cationic lipid at a molar ratio of 30%.

A cationic lipid (SY-3-E14-DMAPr), a non-cationic lipid (DOPE or DPPC), cholesterol or cholesterol analogue (β-sitosterol or stigmastanol) and a PEG-modified lipid (DMG-PEG2K) were dissolved in ethanol and mixed with citrate buffer using a pump system. The instantaneous mixing of the two streams resulted in the formation of empty 4-component lipid nanoparticles through a self-assembly process. The formulation was then subjected to a TFF purification process, removing the citrate buffer and alcohol and exchanging it for storage buffer (10% trehalose). The resulting suspension of preformed empty lipid nanoparticles was then mixed with Firefly Luciferase (FFL) mRNA to encapsulate the FFL mRNA according to methods known in the art. The N/P ratio was about 4.

The lipid amount in the tables indicates the total amount of lipids per 1 mg of mRNA.

TABLE 1A

| | | | | | | Encapsulation |
|---|---|---|---|---|---|---|
| | | | | | | efficiency |
| | Molar ratio of | Molar ratio of | Molar ratio of | Molar ratio | Lipid | before |
| LNP | DMG-PEG2K | SY-3-E14-DMAPr | cholesterol | of DOPE | amount | nebulization |
| number | (%) | (%) | (%) | (%) | (mg) | (%) |
| 1 | 5 | 50 | 35 | 10 | 18.3 | 96 |
| 2 | 5 | 50 | 32.5 | 12.5 | 18.5 | 97 |
| 3 | 5 | 50 | 30 | 15 | 19 | 97 |
| 4 | 5 | 50 | 27.5 | 17.5 | 19 | 96 |
| 5 | 5 | 55 | 30 | 10 | 17 | 96 |
| 6 | 5 | 55 | 27.5 | 12.5 | 17.3 | 97 |
| 7 | 5 | 55 | 25 | 15 | 17.5 | 98 |
| 8 | 5 | 55 | 22.5 | 17.5 | 17.7 | 98 |
| 9 | 5 | 60 | 25 | 10 | 16 | 96 |
| 10 | 5 | 60 | 22.5 | 12.5 | 16.3 | 96 |
| 11 | 5 | 60 | 20 | 15 | 16.5 | 97 |
| 12 | 5 | 40 | 25 | 30 | 24 | 95 |
| (control) | | | | | | |

TABLE 1B

| | | | | | | Encapsulation |
|---|---|---|---|---|---|---|
| | | | | | | efficiency |
| | Molar ratio of | Molar ratio of | Molar ratio of | Molar ratio | Lipid | before |
| LNP | DMG-PEG2K | SY-3-E14-DMAPr | β-Sitosterol | of DOPE | amount | nebulization |
| number | (%) | (%) | (%) | (%) | (mg) | (%) |
| 13 | 5 | 40 | 25 | 30 | 24 | 94 |
| (control) | | | | | | |
| 14 | 5 | 50 | 30 | 15 | 19 | 95 |

TABLE 1C

| | | | | | | Encapsulation |
|---|---|---|---|---|---|---|
| | | | | | | efficiency |
| | Molar ratio of | Molar ratio of | Molar ratio of | Molar ratio | Lipid | before |
| LNP | DMG-PEG2K | SY-3-E14-DMAPr | Stigmastanol | of DOPE | amount | nebulization |
| number | (%) | (%) | (%) | (%) | (mg) | (%) |
| 15 | 5 | 40 | 25 | 30 | 24 | 94 |
| (control) | | | | | | |
| 16 | 5 | 50 | 30 | 15 | 19 | 95 |

TABLE 1D

| | Molar | Molar | | | Encapsulation |
|---|---|---|---|---|---|
| | ratio of | ratio of | Molar | | efficiency |
| | DMG- | SY-3- | ratio of | Molar | before |
| LNP | PEG2K | E14-DMAPr | cholesterol | ratio of | nebulization |
| number | (%) | (%) | (%) | DPPC (%) | (%) |
| 17 | 5 | 40 | 25 | 30 | 94 |
| 18 | 5 | 50 | 30 | 15 | 98 |

TABLE 1E

| | Molar | Molar | | | Encapsulation |
|---|---|---|---|---|---|
| | ratio | ratio of | Molar | | efficiency |
| | of DMG- | SY-3-E14 | ratio of β- | Molar | before |
| LNP | PEG2K | DMAPr | Sitosterol | ratio of | nebulization |
| number | (%) | (%) | (%) | DPPC (%) | (%) |
| 19 | 5 | 50 | 30 | 15 | 96 |

TABLE 1F

| | Molar | Molar | | | Encapsulation |
|---|---|---|---|---|---|
| | ratio of | ratio of | Molar | | efficiency |
| | DMG- | SY-3-E14- | ratio of | Molar | before |
| LNP | PEG2K | DMAPr | Stigmastanol | ratio of | nebulization |
| number | (%) | (%) | (%) | DPPC (%) | (%) |
| 20 | 5 | 50 | 30 | 15 | 96 |

### Example 2. Improving the Nebulization Output Rate of Lipid Nanoparticles

This example demonstrates that reducing the molar ratio of the non-cationic lipid in a lipid nanoparticle encapsulating an mRNA relative to the other lipids in the lipid component improves the nebulization output rate when the lipid nanoparticle is nebulized.

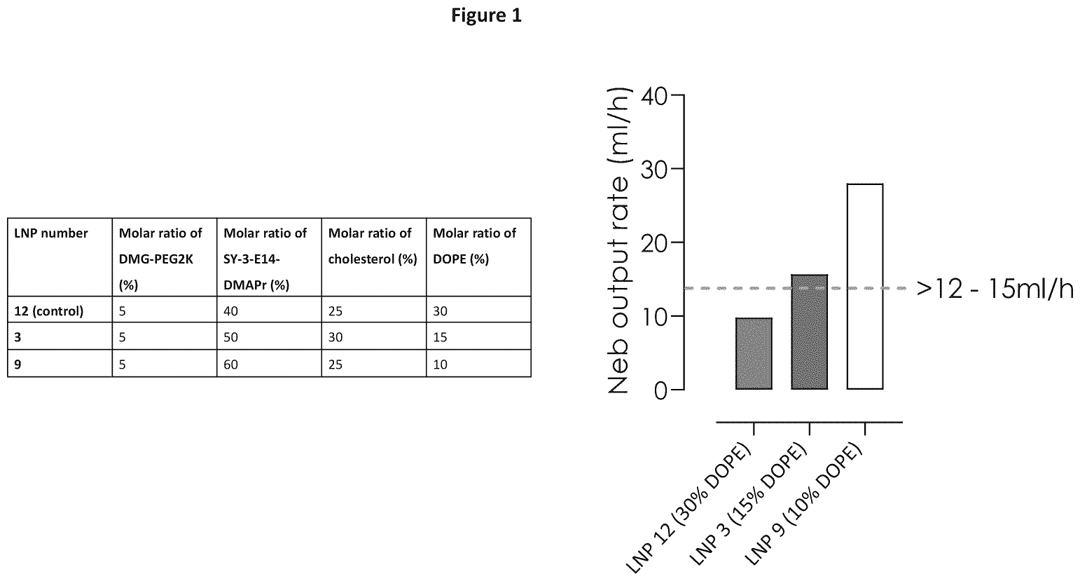

The effect of the molar ratio of the non-cationic lipid in the test lipid nanoparticles prepared in Example 1 on the nebulization output rate (ml/h) was investigated. Nebulization was performed with a nebulizer comprising vibrating mesh technology (VMT). The specific nebulizer model used in this experiment was an Aerogen Solo. 1-6 ml of each test formulation comprising 0.4-0.8 mg/ml mRNA in 4%-10% disaccharide was nebulized. Mice served as test animals for in vivo expression experiments. The results of the experiment are shown in FIGS. 1, 4 and 7A.

As can be seen from FIG. 1, lowering the molar ratio of the non-cationic lipid DOPE from 30% (LNP 12) to 15% (LNP 3) in the test lipid nanoparticles improved the nebulization output rate from about 10 ml/to about 15 ml/h.

Further improvements in the nebulization output rate were achieved by reducing the molar ratio of the non-cationic lipid even further from 15% (LNP 3) to 10% (LNP 9). This reduction effectively doubled the nebulization output rate from about 15 m/h to about 30 ml/h.

The effect of reducing the molar ratio of the non-cationic lipid on the nebulization output rate was also investigated using lipid nanoparticles comprising different cholesterol analogues (0-sitosterol and stigmastanol) in place of cholesterol. As can be seen from FIG. 4, a similar improved nebulization output rates was observed for the lipid nanoparticles with lower molar ratios of non-cationic lipid. Lowering the molar ratio of the non-cationic lipid from 30% (LNP 13 and LNP 15) to 15% (LNP 14 and LNP 16) improved the nebulization output rate. This observation was made with both of the cholesterol analogues β-sitosterol and stigmastanol.

The effect of reducing the molar ratio of the non-cationic lipid on the nebulization output rate was further investigated using DPPC as an alternative non-cationic lipid in the lipid nanoparticle. As can be seen from FIG. 7A, lowering the molar ratio of the non-cationic lipid DPPC from 30% to 15% also improved the nebulization output rate. This improvement was observed with cholesterol as well as the cholesterol analogues β-sitosterol and stigmastanol. This indicates that the findings in this example are broadly applicable to lipid nanoparticles with a lipid component consisting of a cationic lipid, non-cationic lipid, a PEG-modified lipid, and cholesterol or a cholesterol analogue.

Thus, this example shows that lipid nanoparticles with low molar ratio of non-cationic lipid relative to the other lipids in the lipid component are particularly suitable for delivering mRNA to a subject via nebulization as such lipid nanoparticles can be nebulized at improved nebulization output rates. These improved lipid nanoparticles can therefore b^{e} more effective at delivering mRNA to the lungs in a therapeutic setting. Reduced nebulization times provide a significant benefit to patients who are treated with mRNA therapy that is delivery via pulmonary administration.

### Example 3. Maintaining Encapsulation Efficiency after Nebulization

This example demonstrates that reducing the molar ratio of the non-cationic lipid in a lipid nanoparticle encapsulating an mRNA relative to the other lipids in the lipid component maintains or improves the encapsulation efficiency after nebulization of the lipid nanoparticle.

the effect of the molar ratio of the non-cationic lipid in the lipid nanoparticles prepared in Example 1 on the encapsulation efficiency of the lipid nanoparticles after nebulization was investigated.

The encapsulation efficiency of the lipid nanoparticles before nebulization was determined according to methods known in the art. Briefly, encapsulation of mRNA was assessed by performing a Ribogreen assay (Invitrogen) both with versus without the presence of 0.1% Triton-X 100, which disrupts the lipid nanoparticle and releases its contents, to provide a percent encapsulation of mRNA within the lipid nanoparticle relative to total mRNA in the sample. This was performed for each lipid nanoparticle composition before and after nebulization to assess the loss in encapsulation efficiency due to nebulization. The loss in encapsulation efficiency after nebulization was calculated as a percentage. The results of the experiment are shown in FIGS. 2, 5 and 7B.

As can be seen from FIG. 2, lowering the molar ratio of the non-cationic lipid from 30% (LNP 12) to 15% (LNP 3) reduced the change in encapsulation efficiency of the lipid nanoparticle after nebulization from more than a 30% loss to less than a 20% loss. Lowering the molar ratio of the non-cationic lipid further from 15% (LNP 3) to 10% (LNP 9) further reduced the loss in encapsulation efficiency of the lipid nanoparticle after nebulization.

The effect of reducing the molar ratio of the non-cationic lipid on the encapsulation efficiency after nebulization was also investigated using two different cholesterol analogues (β-sitosterol and stigmastanol) in place of cholesterol. As can be seen from FIG. 5, a similar reduced loss in encapsulation efficiency after nebulization was observed for the lipid nanoparticles with lower molar ratios of non-cationic lipid. Lowering the molar ratio of the non-cationic lipid from 30% (LNP 13 and LNP 15) to 15% (LNP 14 and LNP 16) reduced the loss in encapsulation efficiency of the lipid nanoparticle after nebulization. This observation was made with both of the cholesterol analogues (i-sitosterol and stigmastanol.

The effect of reducing the molar ratio of the non-cationic lipid on the encapsulation efficiency after nebulization was further investigated using DPPC as the non-cationic lipid. As can be seen from FIG. 7B, lowering the molar ratio of the non-cationic lipid DPPC from 30% to 15% reduces the loss in encapsulation efficiency of the lipid nanoparticle after nebulization. This improvement was observed with cholesterol as well as the cholesterol analogues β-sitosterol and stigmastanol. This indicates that the findings in this example are broadly applicable to lipid nanoparticles with a lipid component consisting of a cationic lipid, non-cationic lipid, a PEG-modified lipid, and cholesterol or a cholesterol analogue.

Thus this example shows that lipid nanoparticles with low molar ratios of non-cationic lipids relative to the other lipids in the lipid component are particularly suitable for delivering mRNA to a subject via nebulization as they retain more mRNA after nebulization. This is significant as a reduction in encapsulation efficiency after nebulization means that less mRNA is encapsulated by the lipid nanoparticle and so less mRNA will be delivered intact to the lungs to induce expression of the mRNA-encoded protein. The improved lipid nanoparticles are therefore expected to be more effective at delivering intact mRNA to the lungs, wherein it can be expressed.

### Example 4. Expression of Firefly Luciferase (FFL) mRNA in the Lungs

This example demonstrates that reducing the molar ratio of the non-cationic lipid in a lipid nanoparticle encapsulating an mRNA relative to the other lipids in the lipid component can result in improved in vivo expression of the protein encoded by the mRNA when the nebulized lipid nanoparticle composition is delivered in the lungs of a test animal.

The effect of the molar ratio of the non-cationic lipid in the lipid nanoparticles prepared in Example 1 on protein expression in the lungs was investigated.

The lipid nanoparticles comprising FFL mRNA were administered to mice via nebulization. At approximately 5 hours post-dose, the animals were dosed with luciferin by intraperitoneal injection and all animals were imaged using an IVIS imaging system to measure luciferase production in the lung. The results of the experiment are shown in FIGS. 3, 6 and 7C.

As can be seen from FIG. 3, lowering the molar ratio of the non-cationic lipid from 30% (LNP 12) to 15% (LNP 3) and 10% (LNP 9) improved the amount of mRNA that is delivered and thus expressed in the lungs.

The effect of reducing the molar ratio of the non-cationic lipid on protein expression in the lungs was also investigated using two different cholesterol analogues (β-sitosterol and stigmastanol) in place of cholesterol. As can be seen from FIG. 6, lowering the molar ratio of the non-cationic lipid from 30% (LNP 12, LNP 13 and LNP 15) to 15% (LNP 3, LNP 14 and LNP 16) improved the amount of mRNA that is delivered and thus expressed in the lungs. This observation was observed with both of the cholesterol analogues β-sitosterol and stigmastanol.

The effect of reducing the molar ratio of the non-cationic lipid on protein expression in the lungs was further investigated using DPPC as the non-cationic lipid. As can be seen from FIG. 7C, lowering the molar ratio of the non-cationic lipid DPPC from 30% to 15% improves the amount of mRNA that is delivered and thus expressed in the lungs. This improvement was observed with cholesterol as well as the cholesterol analogues β-sitosterol and stigmastanol. This indicates that the findings in this example are broadly applicable to lipid nanoparticles with a lipid component consisting of a cationic lipid, non-cationic lipid, a PEG-modified lipid, and cholesterol or a cholesterol analogue.

Thus this example shows that lipid nanoparticles with low-molar ratios of non-cationic lipids relative to the other lipids in the lipid component are particularly suitable for delivering mRNA to a subject via nebulization as they achieve increased expression levels of the mRNA encoded in the lungs, as is expected in view of the findings made in Examples 2 and 3.

### Example 5. Optimizing the Overall Lipid Content for Nebulization

This example demonstrates that reducing the overall lipid content of a lipid nanoparticle relative to the encapsulated mRNA can result in improved in vivo expression of the protein encoded by the mRNA after pulmonary delivery while also improving nebulization output rate and post-nebulization encapsulation efficiency.

The experiments described in Examples 2-4 were repeated with the lipid nanoparticle formulations shown in Table 2A. In addition to varying the molar ratio of the non-cationic lipid, different molar ratio of the cationic lipid were also tested. The cationic lipid is important for effective encapsulation of the mRNA into lipid nanoparticles, as well as the endosomal release of the mRNA after a lipid nanoparticle has been taken up by a target cell. Both encapsulation efficiency and endosomal release impact the potency of the lipid nanoparticle, i.e. its ability to effectively deliver intact mRNA to induce expression of the mRNA-encoded protein in vivo.

The lipid nanoparticle formulations were prepared as described in Example 1. The resulting lipid nanoparticles were nebulized with a vibrating mesh nebulizer as described in Example 2. The nebulization output rate was measured, and the results are shown in FIG. 8A. As can be seen from the first three bars in this figure, reducing the molar concentration of the cationic lipid from 30% to 25% to 15%, while keeping the molar concentration of the cationic lipid constant at 40% resulted in an increase of the nebulization output rate. However, no improvement of the encapsulation efficiency was observed post-nebulization (see the first three bars in FIG. 8B).

TABLE 2A

| | | | | Total lipid |
|---|---|---|---|---|
| Lipid mol % | | | | weight |
| DMG-PEG2k | SY-3-E14-DMAPR | Chol | DOPE | (mg) |
| 5 | 40 | 25 | 30 | 23.8 |
| 5 | 40 | 30 | 25 | 23.3 |
| 5 | 40 | 40 | 15 | 22.2 |
| 5 | 50 | 35 | 10 | 18.3 |
| 5 | 50 | 32.5 | 12.5 | 18.5 |
| 5 | 50 | 30 | 15 | 18.7 |
| 5 | 50 | 27.5 | 17.5 | 19 |
| 5 | 60 | 25 | 10 | 16.1 |
| 5 | 60 | 22.5 | 12.5 | 16.3 |
| 5 | 60 | 20 | 15 | 16.49 |

Increasing the cationic lipid content to above 40% (molar ratio) by reducing the non-cationic lipid content to 18% or less (molar ratio) in order to arrive at a total lipid:mRNA ratio (mg:mg) of 19:1 or less maintained the improvements in nebulization output rate, but resulted in a dramatic increase in post-nebulization encapsulation efficiency (see FIG. 8B; bars 4-10). The improvement in maintaining effective encapsulation of the mRNA post-nebulization was mirrored at least in part by an improved in vivo potency of the lipid nanoparticle, as shown in FIG. 8C (see bars 4-6 in particular).

Surprisingly, this result was achieved by reducing the total lipid content of the lipid nanoparticle per gram of delivered mRNA. Surprisingly, none of the lipid nanoparticles with reduced total lipid content was inferior in its in vivo potency relative to particles with higher lipid content, despite maintaining better encapsulation efficiency and despite being more efficiently nebulized. Indeed, as can be seen from the last column in Table 2A, lipid nanoparticle formulations with a total lipid:mRNA ratio (mg:mg) of 19:1 or less and a molar ratio of the cationic lipid of greater than 40% were better on all three parameters (nebulization output rate, post-nebulization encapsulation efficiency, and in vivo potency). The lipid nanoparticles with low lipid content routinely achieved nebulization output rates of 12 ml/h or more, while the change in encapsulation efficiency before and after nebulization was typically about 10% or less.

In order to better understand whether the findings with the lipid nanoparticle formulations of Table 2A, which used DOPE as the non-cationic lipid component, are broadly applicable, a second series of lipid nanoparticle formulations comprising different non-cationic lipids were tested. The test formulations of this second series are shown in Table 2B.

TABLE 2B

| | Lipid mol % | | | | | Total |
|---|---|---|---|---|---|---|
| Formulation | DMG- | SY-3-E14- | | Helper | Helper | lipid |
| ID | PEG2K | DMAPR | Chol | lipid | lipid | (mg) |
| 1 | 5 | 60 | 22.5 | 12.5 | DLPC | 16 |
| 2 | 5 | 50 | 32.5 | 12.5 | DLPC | 18 |
| 3 | 5 | 55 | 30 | 10 | DLPC | 17 |
| 4 | 5 | 55 | 25 | 15 | DLPC | 17 |
| 5 | 5 | 60 | 25 | 10 | DMPC | 16 |
| 6 | 5 | 50 | 35 | 10 | DMPC | 18 |
| 7 | 5 | 50 | 30 | 15 | DMPC | 19 |
| 8 | 5 | 60 | 20 | 15 | DMPC | 16 |
| 9 | 5 | 50 | 35 | 10 | DOPC | 18 |
| 10 | 5 | 50 | 30 | 15 | DOPC | 19 |
| 11 | 5 | 60 | 20 | 15 | DOPC | 17 |
| 12 | 5 | 60 | 25 | 10 | DOPC | 16 |
| 13 | 5 | 55 | 27.5 | 12.5 | DOPC | 17 |

The lipid nanoparticle formulations in Table 2B were prepared as described in Example 1. In place of DOPE as the non-cationic lipid component, either DLPC (12:0 PC), DMPC (14:0PC) or DOPC (18:1PC) were used. In each of the tested lipid formulations, the total lipid:mRNA ratio (mg:mg) was 19:1 or less, the molar ratio of the cationic lipid was greater than 40%. Moreover, in this particular experiment, the non-cationic lipid was less than 18% (molar ratio) of the lipid component.

The resulting lipid nanoparticles were nebulized with a vibrating mesh nebulizer as described above, and the nebulization output rate and post-nebulization encapsulation efficiency of each test formulation was measured. As can be seen from FIG. 9A, the nebulization output rate was greatly improved with each of the tested lipid formulations. In fact, all test formulation exceeded 12 ml/h, and many even exceeded 20 ml/h. Similarly, the change in encapsulation efficiency was typically about 10% or less (see FIG. 9B). While a size increase was observed, all tested lipid nanoparticles maintained a size of less than 150 nm. Typically, the size before and after nebulization ranged from about 50-125 nm (see FIG. 9C).

While the tested PC lipids generally had a reduced in vivo potency, mRNA expression achieved after pulmonary delivery of the lipid nanoparticle formulations to test animals in many cases was comparable to that achieved with the original DOPE test formulation (see FIG. 10). In fact, the use of DMPC in place of DOPE resulted in significantly improved mRNA expression levels relative to the DOPE control in formulations comprising 50% cationic lipid and 10% DMPC (molar ratios).

More pronounced increases in potency were observed when DLPE (12:0PE) was used in place of DOPE. The lipid nanoparticle formulations shown in Table 2C were administered to the lungs of test animals through nebulization using a vibrating mesh nebulizer.

TABLE 2C

| | SY-3-E14- | | | | Total |
|---|---|---|---|---|---|
| PEG | DMAPR | Chol | DLPE | Total | lipid |
| mol % | mol % | mol % | mol % | mol % | (mg) |
| 5 | 60 | 22.5 | 12.5 | 100 | 15.9 |
| 5 | 50 | 32.5 | 12.5 | 100 | 18.0 |
| 5 | 55 | 30 | 10 | 100 | 16.7 |
| 5 | 55 | 25 | 15 | 100 | 17.0 |

The results of this experiment are summarized in FIG. 11. The original DOPE-containing lipid nanoparticle formulation was included as positive control. The lipid nanoparticle formulations comprising DLPE as the non-cationic lipid generally performed better than the DOPE-containing control. The best performing formulations had a cationic lipid content of greater than 50% (molar ratio), a non-cationic lipid content of less than 15% (molar ratio), and a total lipid:mRNA ratio (mg:mg) of less than 19:1.

This example demonstrates that the nebulization properties and in vivo potency of an mRNA-encapsulating lipid nanoparticle can be improved by adjusting the total lipid:mRNA ratio (mg:mg) to 19:1 or less. This can be achieved by increasing the molar ratio of the cationic lipid to greater than 40% (molar ratio) and reducing the molar ratio of the non-cationic lipid content. Lipid nanoparticles comprising a cationic lipid at a molar ratio of 18% or less performed particularly well, and in some instances reducing the non-cationic lipid content to less than 15% (molar ratio) resulted in further improvements. The resulting formulations were found to have excellent nebulization output rates and post-nebulization encapsulation efficiencies, resulting in improved in vivo potency as measured by expression levels of the mRNA-encoded proteins in the lung of test animals.

### Example 6. Optimizing the Cationic Lipid:Non-Cationic Lipid Ratio

This example confirms that lipid nanoparticles with a reduced overall lipid content and a molar ratio of the cationic lipid of greater than 40% show significantly higher expression of the mRNA-encoded protein in vivo.

mRNA encoding mCherry protein was encapsulated in lipid nanoparticles having the composition shown in Table 3A.

TABLE 3A

| Lipid mol % | | | | | |
|---|---|---|---|---|---|
| | SY-3- | | | | |
| DMG- | E14- | | | | Total lipid |
| PEG2k | DMAPR | Chol | DOPE | N/P | weight (mg) |
| 5 | 40 | 25 | 30 | 4 | 23.8 |
| 5 | 50 | 30 | 15 | 4 | 18.7 |
| 5 | 60 | 25 | 10 | 4 | 16.1 |

The test formulations were administered to CD-1 mice by nebulization. The various treatment groups are shown in Table 3B. Test animals were sacrificed either immediately after the treatment (baseline) or 24 hours post-exposure. Saline-treated mice served as a control. The lungs were isolated and homogenized, and mCherry expression levels were determined by ELISA.

TABLE 3B

| | | | Dose | | | |
|---|---|---|---|---|---|---|
| | Animals/ | | level | Conc | Dosing | Terminal |
| Group | group | Treatment | (mg) | (mg/mL) | Regimen | time point |
| 1 | 8 | Untreated | n/a | n/a | Untreated | On day 1, 2 |
| | | | | | | (n = 4) |
| 2 | 12 | 40% SY-3- | 24 | 0.6 | For 4 hours | On day 1, |
| | | E14-DMAPR | | | via aerosol | immediately |
| 3 | 12 | 50% SY-3- | 24 | 0.6 | inhalation | after dose |
| | | E14-DMAPR | | | using a | (n = 6/group); |
| 4 | 12 | 60% SY-3- | 24 | 0.6 | nebulizer on | On day 2 at |
| | | E14-DMAPR | | | Day 1* | 24 h post- |
| 5 | 12 | 40% SY-3- | 6 | 0.6 | For 1 hour | exposure |
| | | E14-DMAPR | | | via aerosol | (n = 6/group) |
| 6 | 12 | 50% SY-3- | 6 | 0.6 | inhalation | |
| | | E14-DMAPR | | | using a | |
| 7 | 12 | 60% SY-3- | 6 | 0.6 | nebulizer on | |
| | | E14-DMAPR | | | Day 1* | |

The results of this experiment are summarized in FIG. 12. mCherry expression levels in the lung were highest when a 24 mg dose of mRNA was administered by nebulization over a 4 hour period. Mice treated with lipid nanoparticle formulations comprising 50% or 60% (molar ratio) of the cationic lipid had significantly higher expression levels than mice treated with lipid nanoparticles comprising 40% (molar ratio) of the cationic lipid. Mice in groups 3, 4, 6 and 7 consistently expressed more than 250 ng mCherry protein/mg total lung protein.

Expression levels were higher in groups receiving a 24 mg administered over a 4 hour period in comparison to groups receiving a 6 mg dose over a 1 hour period. Surprisingly, mCherry expression in mice treated with the 24 mg dose of a lipid nanoparticle formulation comprising 50% (molar ratio) of the cationic lipid (group 3) was about 25-fold higher than in mice treated with the same dose of a lipid nanoparticle formulation comprising 40% (molar ratio) of the cationic lipid (group 2). Mice in group 3 averaged about 1.15 ng mCherry protein/mg total protein in their lungs. No mCherry expression was detectable at the chosen 24-hour time point in the group receiving a 6 mg dose of a lipid nanoparticle formulation comprising 40% (molar ratio) of the cationic lipid (group 5). As can be seen from a comparison of groups 3 and 6, there was a clear dose dependency as mice in group 3 had about 3 times more mCherry protein in their lungs than mice in group 6. The difference in expression levels was smaller between the groups of mice that had received a lipid nanoparticle formulation comprising 60% (molar ratio) of the cationic lipid (groups 4 and 7).

This example confirms that increasing the molar ratio of the cationic lipid to greater than 40% (e.g., to 50% or 60%,) while reducing the overall lipid:mRNA ratio to 19:1 or less through a reduction of the non-cationic lipid content, results in lipid nanoparticle formulations with improved in vivo potency. The data also suggest that the optimal molar ratio of the cationic lipid in lipid nanoparticles with a total lipid:mRNA ratio (mg:mg) of 19:1 or less is around about 50%.

### Example 7. Optimized Lipid Formulations have Improved Nebulization Characteristics

This example demonstrates that adjusting the cationic lipid content while reducing the non-cationic lipid content can improve nebulization of a lipid nanoparticle independent of the cationic lipid included in the formulation. Such optimized lipid nanoparticle formulations have improved nebulization characteristics, are better at maintaining post-nebulization encapsulation of the mRNA and have greater in vivo potency.

To assess whether the findings with SY-3-E14-DMAPr are broadly applicable, experiments corresponding to those described in Examples 1-6 were repeated with a lipid nanoparticle formulation comprising a structurally different cationic lipid, TL1-01D-DMA. The compositions of the tested lipid nanoparticles are shown in Table 4A.

TABLE 4

| | Lipid mol % | | | | Total lipid |
|---|---|---|---|---|---|
| Formulation | DMG- | TL1-01D- | | | amount |
| ID | PEG2K | DMA | Chol | DOPE | (mg) |
| 1 | 3 | 40 | 25 | 32 | 20.6 |
| 2 | 3 | 50 | 32 | 15 | 15.6 |
| 4 | 3 | 50 | 30 | 17 | 15.8 |
| 5 | 3 | 50 | 27 | 20 | 16.0 |
| 7 | 3 | 47 | 27.5 | 22.5 | 17.1 |

As can be seen from FIGS. 13A and 13B, adjusting the molar ratios of both the cationic lipid and the non-cationic lipid resulted in improved nebulization output of the resulting lipid nanoparticles, while maintaining encapsulation of the mRNA. Increasing the amount of TL1-01D-DMA to 47% (molar ratio) and reducing the DOPE content to about 22.5% (molar ratio), resulted in lipid nanoparticles with a total lipid to mRNA ratio (mg:mg) of 17.1 which could be more effectively nebulized at an output rate of greater than 15 ml/h while keeping the change in encapsulation efficiency post-nebulization below 10%. This optimized lipid nanoparticle formulation also displayed improved in vivo potency relative to other cationic lipid formulations that had previously been identified as being highly effective for pulmonary delivery via nebulization (see FIG. 13C).

This example confirms that increasing the molar ratio of the cationic lipid to greater than 40% while reducing the overall lipid:mRNA ratio to 19:1 or less through a reduction of the non-cationic lipid content, results in lipid nanoparticle formulations with improved in vivo potency. It also confirms that the optimal molar ratio of the cationic lipid in lipid nanoparticles with a total lipid:mRNA ratio (mg:mg) of 19:1 or less is around about 50%.

### Example 8. Lipid Nanoparticles with PE Lipids Outperform Those with PC Lipids

This example demonstrates that lipid nanoparticles with PE lipids as their non-cationic lipid component generally have improved nebulization characteristics and in vivo potency compared to lipid nanoparticles with PC lipids as their non-cationic lipid component. This example also confirms that lipid nanoparticles with a reduced non-cationic lipid content and thus an overall lower total lipid content perform better during nebulization and result in improved mRNA expression in vivo.

To evaluate the relative performance of PC lipids and PE lipids, the experiments described in Examples 2-4 were repeated with a lipid nanoparticle with DPPC (a PE lipid) as the cationic lipid component. A standard lipid nanoparticle with 30% DOPE as the non-cationic lipid component was included as comparator. The lipid nanoparticles were prepared as described in Example 1. The test formulations are described in Table 5A.

TABLE 5A

| Lipid mol % | | | | | |
|---|---|---|---|---|---|
| | SY-3-E14- | | DOPE or | | Total lipid |
| DMG-PEG2k | DMAPR | Chol | DPPC | N/P | weight (mg) |
| 5 | 40 | 25 | 30 | 4 | 23.8 |
| 5 | 50 | 30 | 15 | 4 | 18.7 |
| 5 | 60 | 25 | 10 | 4 | 16.1 |

Nebulization was performed as described in Example 2, and the nebulization output rate was measured. As expected, decreasing the amount of DPPC from 30% to 15% (molar ratio) resulted in an increase in the nebulization output rate (see FIG. 14A). The decrease in DPPC from 30% to 15% (molar ratio) was accompanied by increasing the percentage of SY-3-E14-DMAPR from 40% to 50%. FIG. 14B also shows that the nebulization output was improved slightly for lipid nanoparticles with 15% DPPC compared to those with 30% DOPE.

Next, the change in encapsulation efficiency was determined for each of the compositions. The relative change in encapsulation efficiency after nebulization was calculated as described in Example 3. As can be seen from FIG. 14B, decreasing the level of DPPC from 30% to 15% improved the post-nebulization encapsulation efficiency. Lipid nanoparticles comprising DPPC as the non-cationic lipid had improved post-nebulization encapsulation efficiencies compared to the standard DOPE-containing lipid nanoparticle.

To evaluate the in vivo potency of the lipid nanoparticles, the expression of luciferase mRNA in the lungs of mice was measured as described in Example 4. In this experiment, a lipid nanoparticle comprising ML2 as the cationic lipid component was included as a further comparator. The results are shown in FIG. 14C. Decreasing the percentage of DPPC from 30% to 15% or 10% (molar ratio), while increasing the relative molar ratio of the cationic lipid from 40% to 50% or 60%, respectively, resulted in an increase in average radiance detected in the lungs of test animals. This finding was consistent with the observations made in the preceding examples, which demonstrated that improved nebulization characteristics associated with lowering the total lipid content of mRNA-encapsulating lipid nanoparticles typically resulted in higher mRNA expression in vivo.

Surprisingly, the data in FIG. 15 also show that standard lipid nanoparticles comprising 30% DOPE as the non-cationic lipid resulted in a significantly higher mRNA expression than optimized lipid nanoparticles comprising 15% or 10% DPPC. To evaluate whether this finding could be extended to other PC lipids and to determine whether PE lipids generally were more potent in inducing mRNA expression in the lungs of test animals, the in vivo potency of various PE and PC lipids was compared. Specifically, the PC lipids DPPC, DSPC and DOPC, and the PE lipids DLPE, DMPE, DLoPE and DOPE were evaluated.

The lipid nanoparticles were produced according to the method described in Example 1. The composition of the test lipid nanoparticle formulations is shown in Table 5B. For this experiment, a non-optimized lipid nanoparticle formulation was used. The results of the experiment are summarized in FIG. 15.

TABLE 5B

| TL1-01D- | mg of TL1- | Helper | Mg of helper | |
|---|---|---|---|---|
| DMA | 01D-DMA per | lipid | lipid per mg of | Total amount of |
| mol % | mg of mRNA | mol % | mRNA | lipid (mg) |
| 40 | 7.8 | 32 | 7.4 | ~21 |

As can be seen from FIG. 15, with the exception of DOPC, administration of lipid nanoparticles with PE lipids as their non-cationic lipid by nebulization resulted in higher mRNA expression levels in the lungs of mice than lipid nanoparticles with PC lipids as their non-cationic lipid.

Consistent with the observations made in the preceding examples, this example confirms that lipid nanoparticles with a reduced non-cationic lipid content and thus an overall lower total lipid:mRNA ratio (19:1 or less) perform better during nebulization and result in improved mRNA expression in vivo. In addition, this example demonstrates that lipid nanoparticles comprising PE lipids as their non-cationic lipid component generally have greater in vivo potency compared to lipid nanoparticles comprising PC lipids as their non-cationic lipid component.

### Example 9. Synthesis of Lipids for Use in Pulmonary Delivery

Synthesis of cationic lipids for use in lipid nanoparticles of the present invention are described in this example.

### 1. GL-TES-SA-DME-E18-2

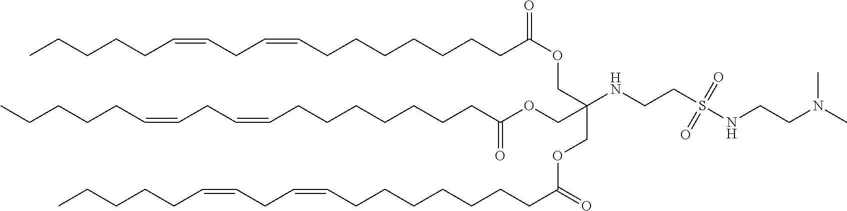 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

### Synthetic Scheme

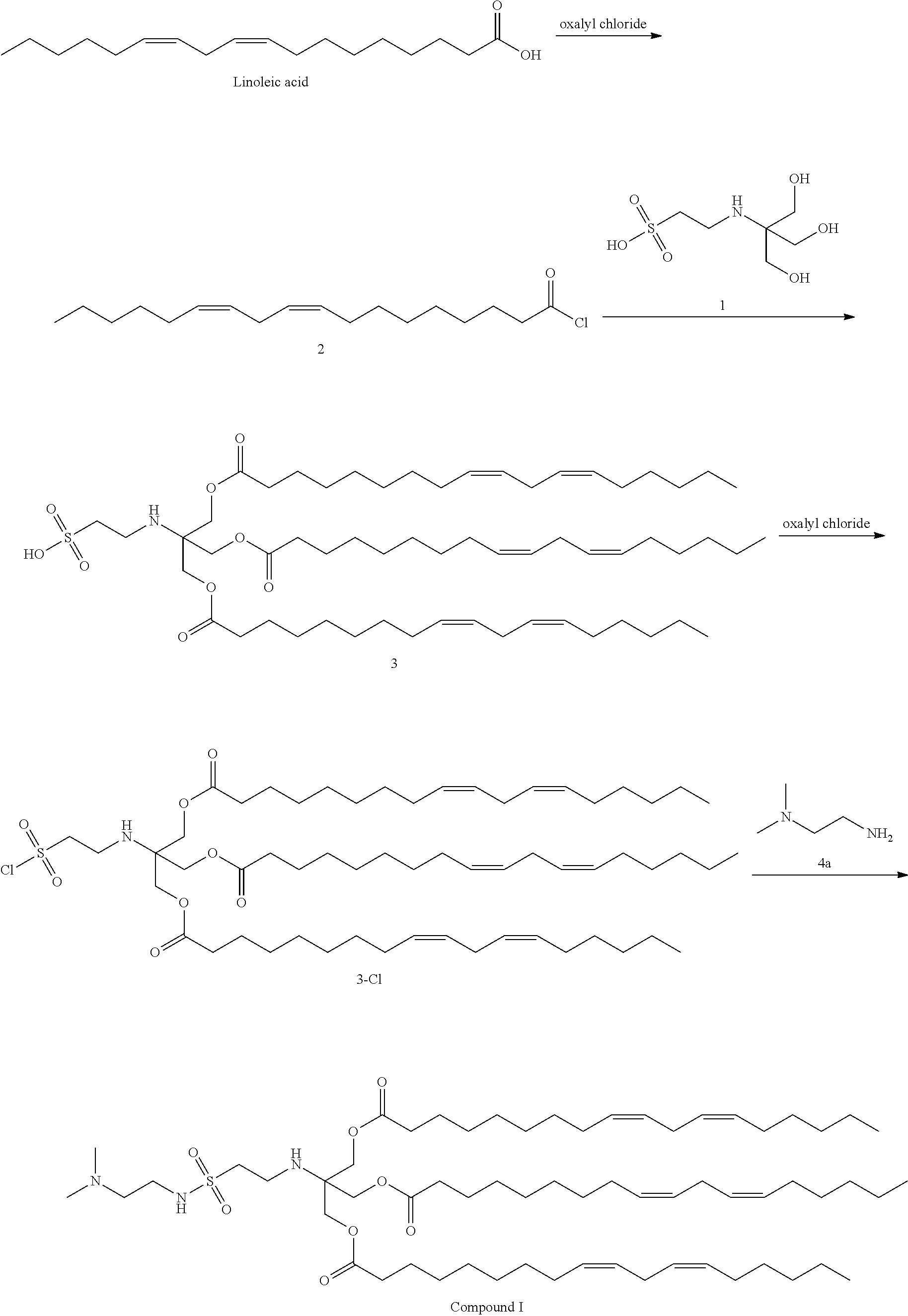

### Synthetic Protocol

### Synthesis of (9Z,12Z)-Octadeca-9,12-dienoyl chloride (2)

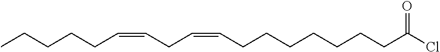 [SMILES: CCCCC/C=C\C/C=C\CCCCCCCC(=O)Cl]

To a solution of Linolenic acid (1.0 g, 3.6 mmol) in 10 mL dichloromethane at 0° C., was added N,N-dimethylformamide (0.1 mL) and oxalyl chloride (1.2 mL, 14.3 mmol). The reaction mixture was warmed to room temperature and stirred for 3 h. The solvent was removed to the under reduced pressure, and the crude was used in next step without further purification.

### Synthesis of 2-((1,3-Bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic acid (3)

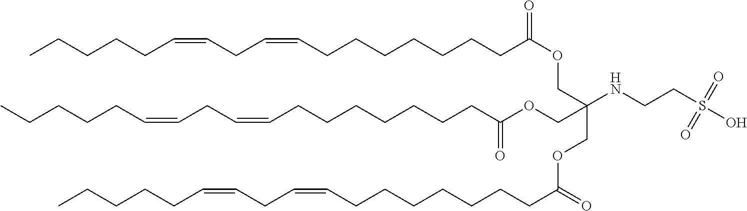 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)O]

To a solution of (9Z,12Z)-octadeca-9,12-dienoyl chloride 2 (1.1 g, 3.6 mmol) in anhydrous N)N-dimethylacetamide (5.0 mL) and N-methyl morpholine (3.0 mL), was added 2-((1,3-dihydroxy-2-(hydroxymethyl)propan-2-yl)amino)ethane-1-sulfonic acid (1, TES) (200 mg, 0.87 mmol). The reaction mixture was heated to 55° C. for 3 h. MS analysis showed the formation of desired product. The reaction mixture was cooled to room temperature, diluted with water (100 mL) and extracted with dichloromethane (2×100 mL). The combined organic layer was washed with saturated brine (100 mL) and dried over anhydrous sodium sulfate. The solvent was removed under vacuum, and the residue was purified by column chromatography (40 g SiO₂: 0 to 10% methanol in dichloromethane gradient) to obtain 2-((1,3-bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic acid as colorless solid (562 mg, 47% yield).

### Synthesis of 2-((2-(Chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) (3-Cl)

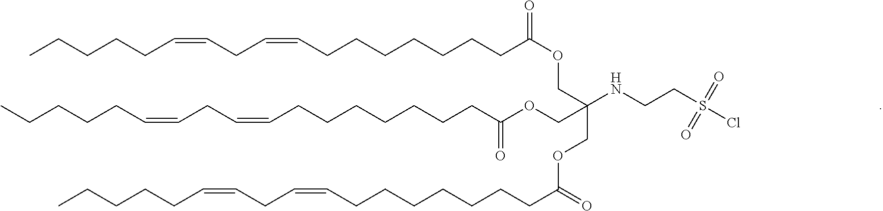 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)Cl]

To a solution of 2-((1,3-bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic acid 3 (210 mg, 0.82 mmol) in anhydrous dichloromethane (5.0 mL) at 0° C. was added N, N-dimethylformamide (0.05 mL) and oxalyl chloride (0.08 mL, 2.1 mmol). The reaction mixture was warmed to room temperature and stirred for 3 h. The solvent was removed to the dryness under reduced pressure to give 2-((2-(chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate), which was used in next step without further purification.

### Synthesis of 2-((2-(N-(2-(dimethylamino)ethyl)sulfamoyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) (Compound I)

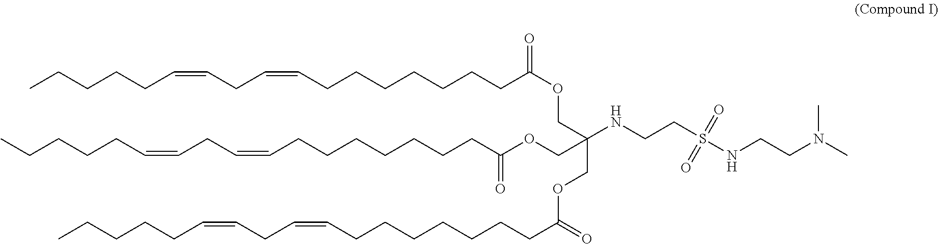 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

To a solution of 2-((2-(chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) 3-Cl (210 mg, 0.82 mmol) in anhydrous dichloromethane (5.0 mL) at 0° C. was added N,N′-dimethylethane-1,2-diamine (182 mg, 2.1 mmol). The reaction mixture was warmed to room temperature and stirred for 3 h. The reaction was quenched by addition of water, and the mixture was extracted with dichloromethane (2×100 mL). The combined organic layer was washed with saturated brine (100 mL) and dried over anhydrous sodium sulfate. The solvent was removed, and the crude was purified by column chromatography (40 g SiO₂: 0 to 15% methanol in dichloromethane gradient) to obtain 2-((2-(N-(2-(dimethylamino)ethyl)sulfamoyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) as yellow oil (139 mg, 62% yield).

1H NMR (300 MHz, Chloroform-d) δ 5.26-5.44 (m, 12H), 4.09 (s, 6H), 3.06-3.18 (m, 6H), 2.75 (t, 6H), 2.47 (t, 2H), 2.32 (t, 6H), 2.24 (s, 6H), 2.00-2.10 (m, 12H), 1.52-1.65 (m, 4H), 1.20-1.40 (m, 44H), 0.88 (t, 9H).

APCI-MS analysis: Calculated C64H115N3O8S, [M+H]=1186.7, observed=1186.8.

### 2. GL-TES-SA-DMP-E18-2

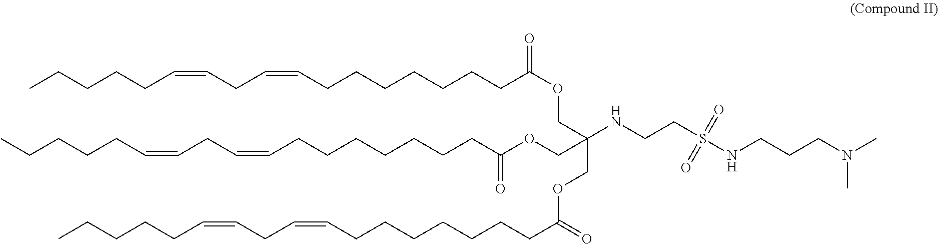 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

### Synthetic Pathway

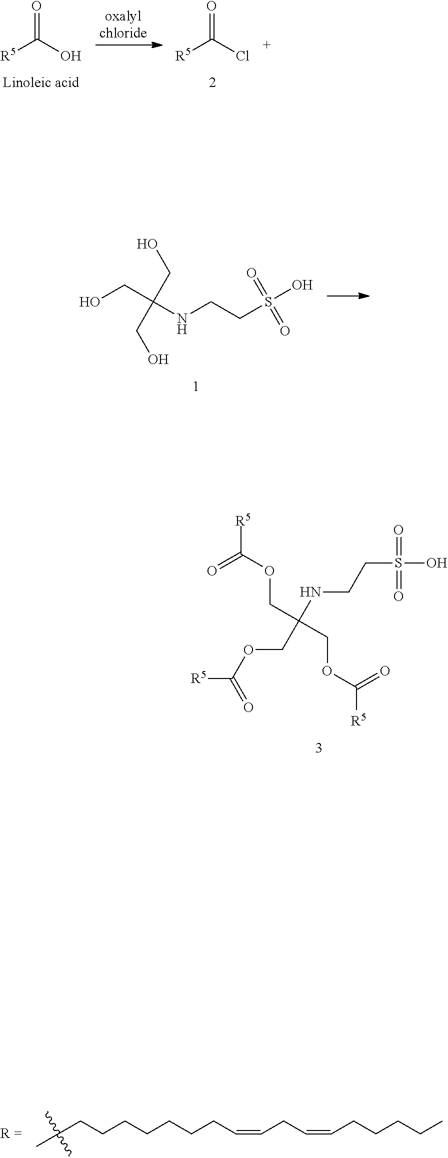

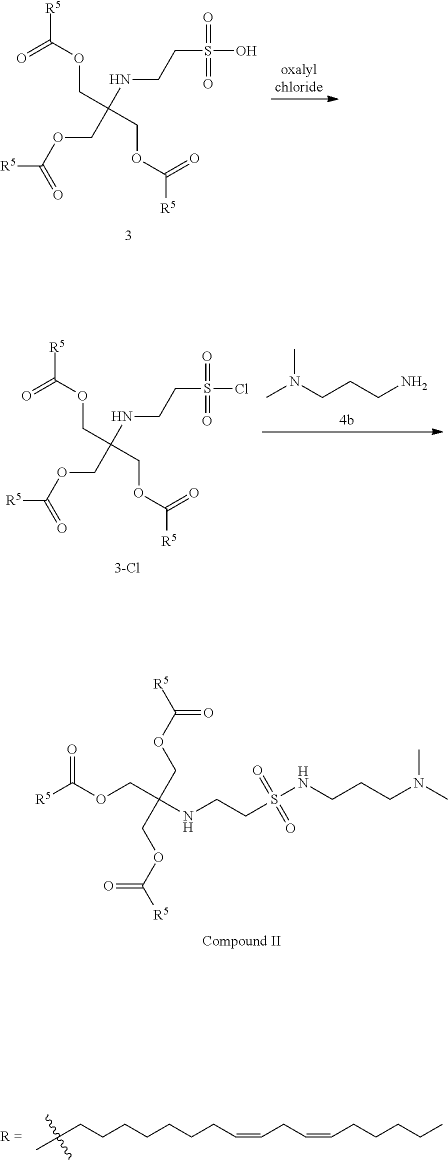

### Synthetic Protocol

Compound II was prepared following the above representative procedure in similar yields to those obtained for Compound I.

Linoleic acid is treated with a chlorinating reagent such as oxalyl chloride to provide the acyl chloride compound 2. Reaction of compound 2 with a nucleophilic compound, such as the buffer compound 1, affords compound 3. Compound 3 is treated with a chlorinating agent such as oxalyl chloride to provide the electrophilic compound 3-Cl. Reaction of 3-Cl with a nucleophile such as compound 4b then affords compound II.

The reaction conditions used were as follows:

| | | Reaction | | |
|---|---|---|---|---|
| SM | Product | Conditions | Scale | Yield |
| Linoleic | 2 | Oxalyl chloride, | 1.0 g of | — |
| acid | | DMF DCM | linoleic acid | |
| 1 & 2 | 3 | Dimethylacetamide + | 200 mg of 1 | 562 mg |
| | | N-Methylmorpholine | | 47% yield |
| 3 | 3-Cl | Oxalyl chloride, | 200 mg of 3 | — |
| | | DMF DCM | | |
| 3-Cl & | Compound | DCM | 200 mg of | 105 mg |
| 4b | II | | 3-Cl | (49% over |
| | | | | 2 steps) |

1H NMR (300 MHz, Chloroform-d) δ 5.24-5.42 (m, 12H), 4.08 (s, 6H), 3.17 (t, 2H), 3.06 (bs, 4H), 2.75 (t, 6H), 2.43 (t, 2H), 2.31 (t, 6H), 2.23 (s, 6H), 1.98-2.08 (m, 12H), 1.70 (quint, 2H), 1.52-1.63 (m, 4H), 1.17-1.45 (m, 44H), 0.87 (t, 9H).

APCI-MS analysis: Calculated C65H117N3O8S, [M+H]=1100.7, observed=1100.8.

### 3. TL1-01D-DMA

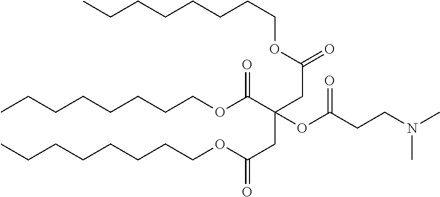 [SMILES: CCCCCCCCOC(=O)CC(CC(=O)OCCCCCCCC)(OC(=O)CCN(C)C)C(=O)OCCCCCCCC]

### Synthetic Scheme

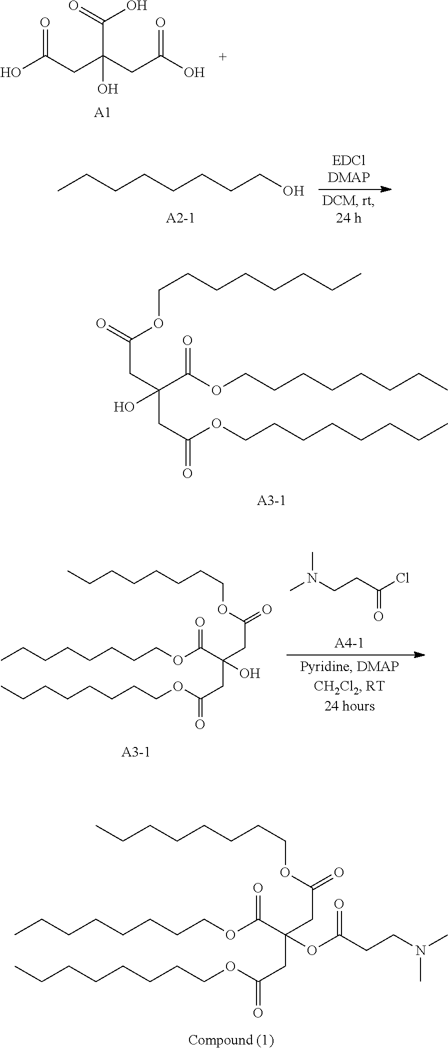

### Synthetic Protocol

### Synthesis of (trioctyl 2-hydroxypropane-1,2,3-tricarboxylate)

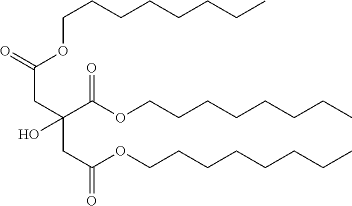 [SMILES: CCCCCCCCOC(=O)CC(O)(CC(=O)OCCCCCCCC)C(=O)OCCCCCCCC]

To a solution of citric acid A1 (2.1 g, 11.0 mmol) and 1-octanol A2-1 (9.4 g, 72.6 mmol) in dichloromethane (40 mL), DMAP (1.34 g, 11.0 mmol) and EDCI (14.3 g, 72.6 mmol) were added, and the resulting mixture was stirred at room temperature 24 h. The reaction mixture was evaporated under vacuum. The residue was dissolved in dichloromethane (200 mL) and washed with brine (100 mL×3). After dried over anhydrous Na₂SO₄, the solvent was evaporated, and the crude was purified by column chromatography (220 g SiO₂: 0 to 20% ethyl acetate in hexane gradient) to obtain (trioctyl 2-hydroxypropane-1,2,3-tricarboxylate) as colorless oil (5.2 g, 90%).

### Synthesis of (trioctyl 2-((3-(dimethylamino)propanoyl)oxy)propane-1,2,3-tricarboxylate)

To a solution of trioctyl 2-hydroxypropane-1,2,3-tricarboxylate A3-1 (0.528 g, 1.0 mmol), DMAP (122 mg, 1.0 mmol) and pyridine (316 mg, 4.0 mmol) in 10 mL dichloromethane, 3-(dimethylamino)propanoyl chloride A4-1 (271 mg, 2.0 mmol) was added at 0° C., and then the resulting mixture was stirred at room temperature for 24 h. The reaction mixture was evaporated under vacuum. The residue was dissolved in dichloromethane (100 mL) and washed with brine (80 mL×3). After dried over anhydrous Na₂SO₄, the solvent was evaporated, and the crude was purified by column chromatography (80 g SiO₂: 0 to 10% methanol in dichloromethane gradient) to obtain trioctyl 2-((3-(dimethylamino)propanoyl)oxy)propane-1,2,3-tricarboxylate as colorless oil (210 mg, 33%).

Alternatively, to a suspension of 3-(dimethylamino)propanoic acid (8.02 g, 68.5 mmol) in 150 mL dichloromethane, was added EDCI (13.1 g, 68.5 mmol) and DMAP (2.09 g, 17.1 mmol) at 0° C., and the resulting mixture was stirred at this temperature for 5 min. A solution of trioctyl 2-hydroxypropane-1,2,3-tricarboxylate A3-1 (9.05 g, 17.1 mmol) in 10 mL dichloromethane was added, and then the resulting mixture was stirred at room temperature for 48 h. The reaction mixture was diluted with dichloromethane, washed with saturated sodium bicarbonate and brine. After dried over sodium sulfate, the organic layer was evaporated under vacuum. The residue was purified by column chromatography (220 g SiO₂: 0 to 10% methanol in dichloromethane gradient) to obtain trioctyl 2-((3-(dimethylamino)propanoyl)oxy)propane-1,2,3-tricarboxylate as colorless oil (4.2 g, 38%).

¹H NMR (300 MHz, CDCl₃) δ 4.56 (s, br., 6H), 4.24 (t, 2H), 4.12 (s, 2H), 2.55 (t, 2H), 2.28-2.17 (m, 14H), 1.63-1.48 (m, 8H), 1.25 (s, br., 32H), 0.86 (t, 12H).

APCI-MS analysis: Calculated C35H65NO8, [M+H]=627.9, Observed=628.5.

### 4. TL1-04D-DMA

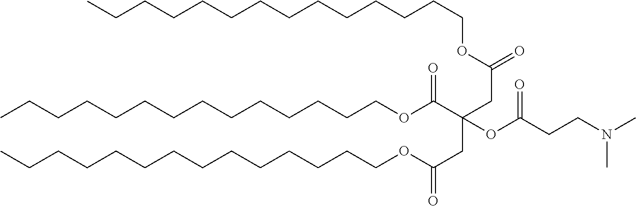 [SMILES: CCCCCCCCCCCCCCOC(=O)CC(CC(=O)OCCCCCCCCCCCCCC)(OC(=O)CCN(C)C)C(=O)OCCCCCCCCCCCCCC]

TD1-04D-DMA can be made in a similar manner as TD-01D-DMA, which is described above.

### 5. SY-3-E14-DMAPr

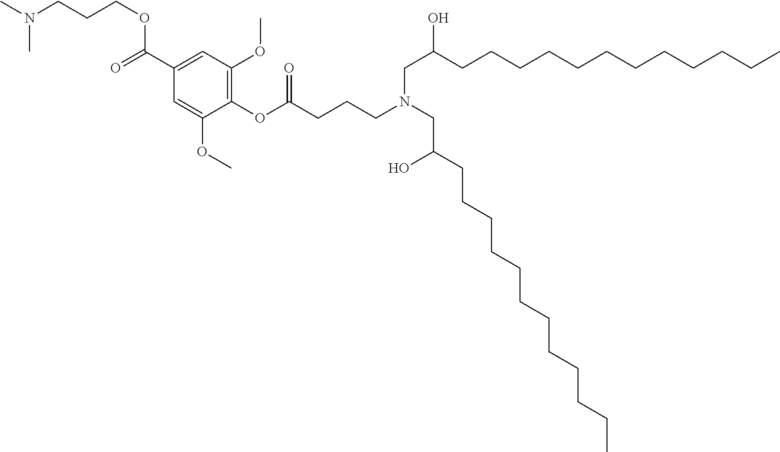 [SMILES: CCCCCCCCCCCCC(O)CN(CCCC(=O)Oc1c(OC)cc(C(=O)OCCCN(C)C)cc1OC)CC(O)CCCCCCCCCCCC]

### Synthetic Scheme

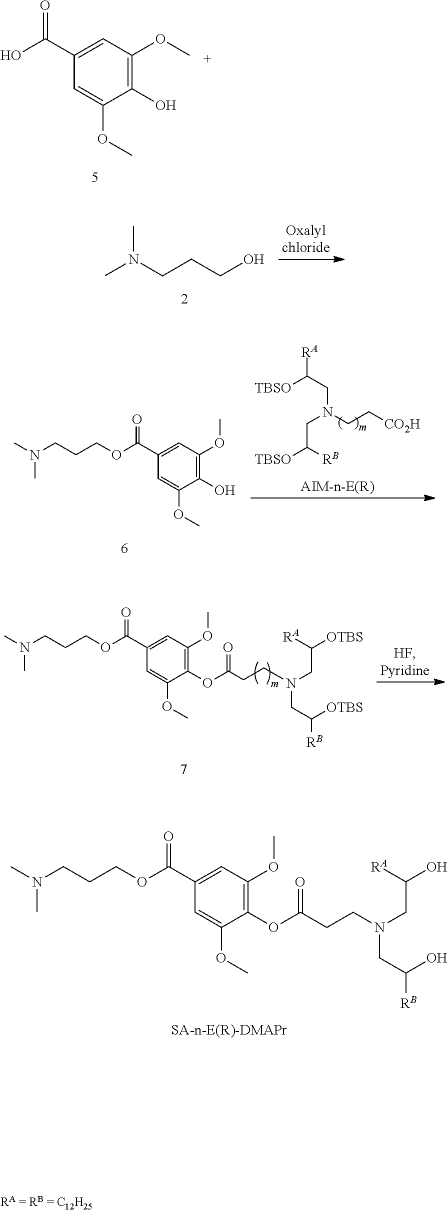

### Synthesis of 3-(Dimethylamino)propyl 4-hydroxy-3,5-dimethoxybenzoate (6)

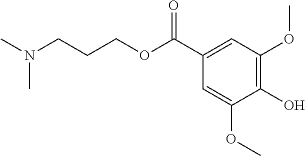 [SMILES: COc1cc(C(=O)OCCCN(C)C)cc(OC)c1O]

To a suspension of syringic acid 5 (7.5 g, 0.04 moles) in 100 mL dichloromethane at 0° C. was added oxalyl chloride (12.8 mL, 0.15 mole) followed by dimethylformamide (5 drops), and the resulting mixture was stirred for 2 h at this temperature. The reaction mixture was evaporated to dryness, and the residue was dissolved in 100 mL dichloromethane. After cooling to 0° C., 3-(dimethylamino)propan-1-ol 2 (4.5 mL, 40 mmol) was added slowly, and the reaction mixture was stirred at room temperature overnight. The precipitate was filtered to give 3-(dimethylamino)propyl 4-hydroxy-3,5-dimethoxybenzoate 6 as white solid (6.2 g, 58%).

### 6. TL1-10D-DMA

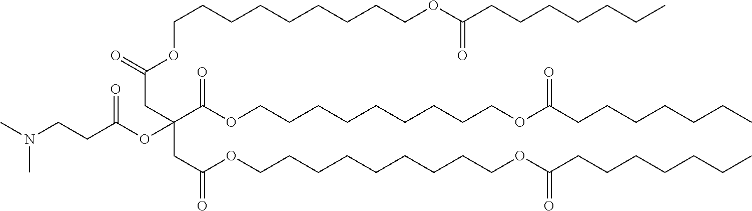 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

TD1-04D-DMA can be made in a similar manner as TD-01D-DMA, which is described above.

### 7. HEP-E3-E10

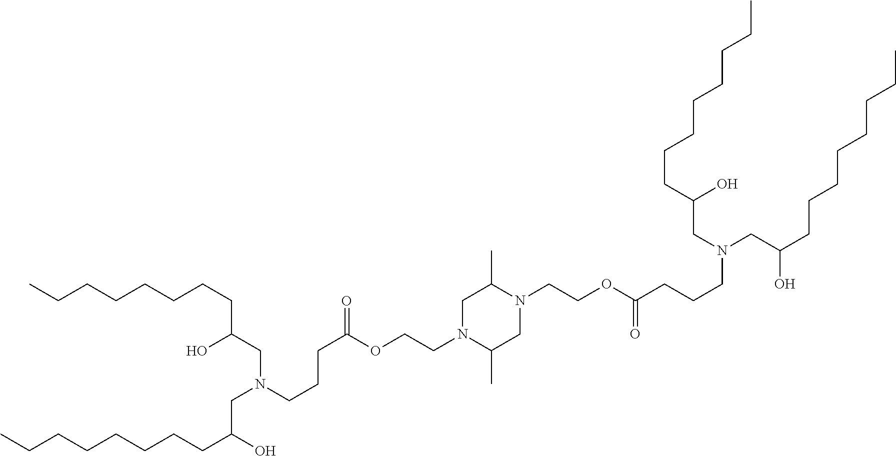 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

### Synthetic Scheme

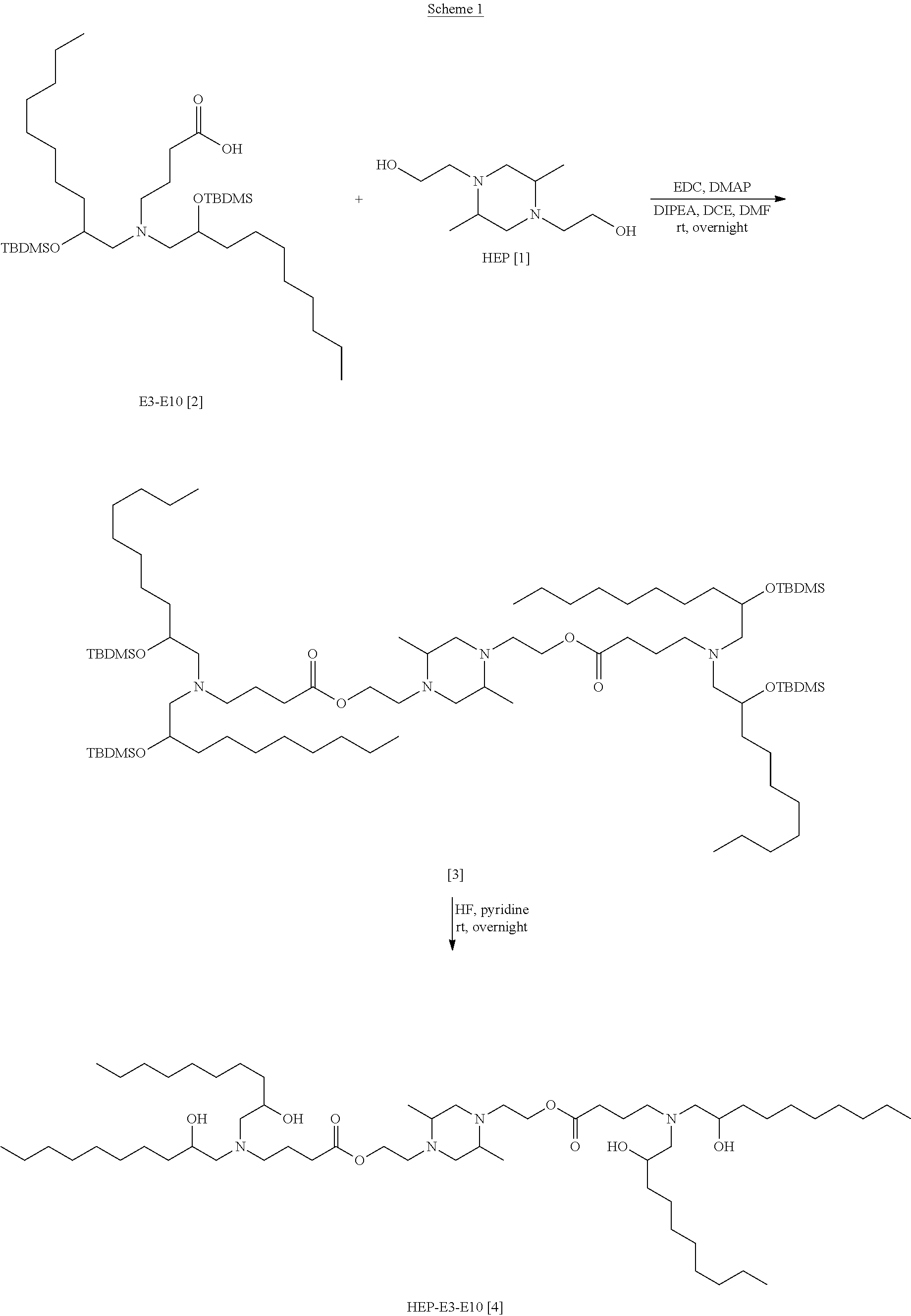 [SMILES: CCCCCCCCC(CN(CCCC(=O)O)CC(CCCCCCCC)O[Si](C)(C)C(C)(C)C)O[Si](C)(C)C(C)(C)C ; CC1CN(CCO)C(C)CN1CCO ; CCCCCCCCC(CN(CCCC(=O)OCCN1CC(C)N(CCOC(=O)CCCN(CC(CCCCCCCC)O[Si](C)(C)C(C)(C)C)CC(CCCCCCCC)O[Si](C)(C)C(C)(C)C)CC1C)CC(CCCCCCCC)O[Si](C)(C)C(C)(C)C)O[Si](C)(C)C(C)(C)C ; CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

### Synthetic Protocol

### Synthesis of [3]

As set out in Scheme 1: To a solution containing HEP [11 (0.100 g, 0.494 mmol, 1.0 eq), E3-E 10 [2](0.668 g, 1.038 mmol, 2.1 eq), 1 ml of dimethylformamide, 3 ml of dichloroethane, diisopropylethylamine (0.344 μL, 1.98 mmol, 4.0 eq), and N,N-Dimethylaminopyridine (0.024 g, 0.198 mmol, 0.4 eq) was added 1-Ethyl-3-(3-dimethylaminopropyl)carbodiimide (0.285 g, 1.48 mmol, 3.0 eq) and allowed to react at room temperature overnight (18 hr). Afterwards, the reaction mixture was concentrated using a rotavapor and purified using a Buchi Combi-flash system on 12 g, 40 μm-sized silica gel columns using hexanes/ethyl acetate as the mobile phase, yielding a colorless oil (70% yield).

### Synthesis of HEP-E3-E10 14]

As set out in Scheme 1: To a 20 ml Polypropylene scintillation vial equipped with a PTFE stir-bar was added 131 (0.500 g, 0.344 mmol, 1.0 eq) along with 4 ml of dry tetrahydrofuran. The vial was cooled to 0-5° C. on an ice bath and HF/pyridine (1.76 ml, 67.86 mmol, 197.3 eq) was added dropwise. After addition, the reaction vial was allowed to warm to room temperature and stirred overnight (18 hr). Afterwards, the reaction mixture was neutralized with saturated sodium bicarbonate at 0° C. Ethyl acetate was used for extraction (3×). The organic layers were combined, washed with saturated sodium chloride (4×), dried with sodium sulfate, filtered, and rotovaped to yield an off-yellow oil. This oil was further purified using a Buchi Combi-flash system on 12 g, 40 μm-sized silica gel columns using dichloromethane/methanol (3% methanol) as the mobile phase, yielding a colorless oil (60% yield).

1H NMR (400 MHz, CDCl3) 4.16 (m, 4H), 3.60 (m, 4H), 2.97 (m, 3H), 2.78 (d, 3H), 2.58 (m, 9H), 2.37 (m, 12H), 2.15 (m, 2H), 1.78 (m, 4H), 1.44 (m, 7H), 1.36 (m, 9H), 1.26 (br, 45H), 1.05 (d, 6H), 0.87 (t, 12H).

Expected M/Z=998.59, Observed=998.0.

### 8. HEP-E4-E10

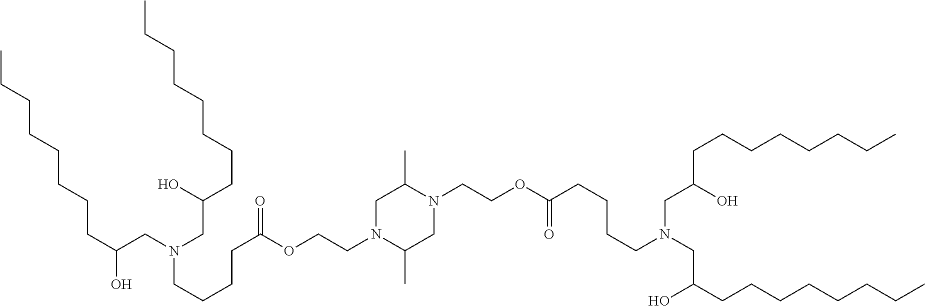 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

### Synthetic Scheme

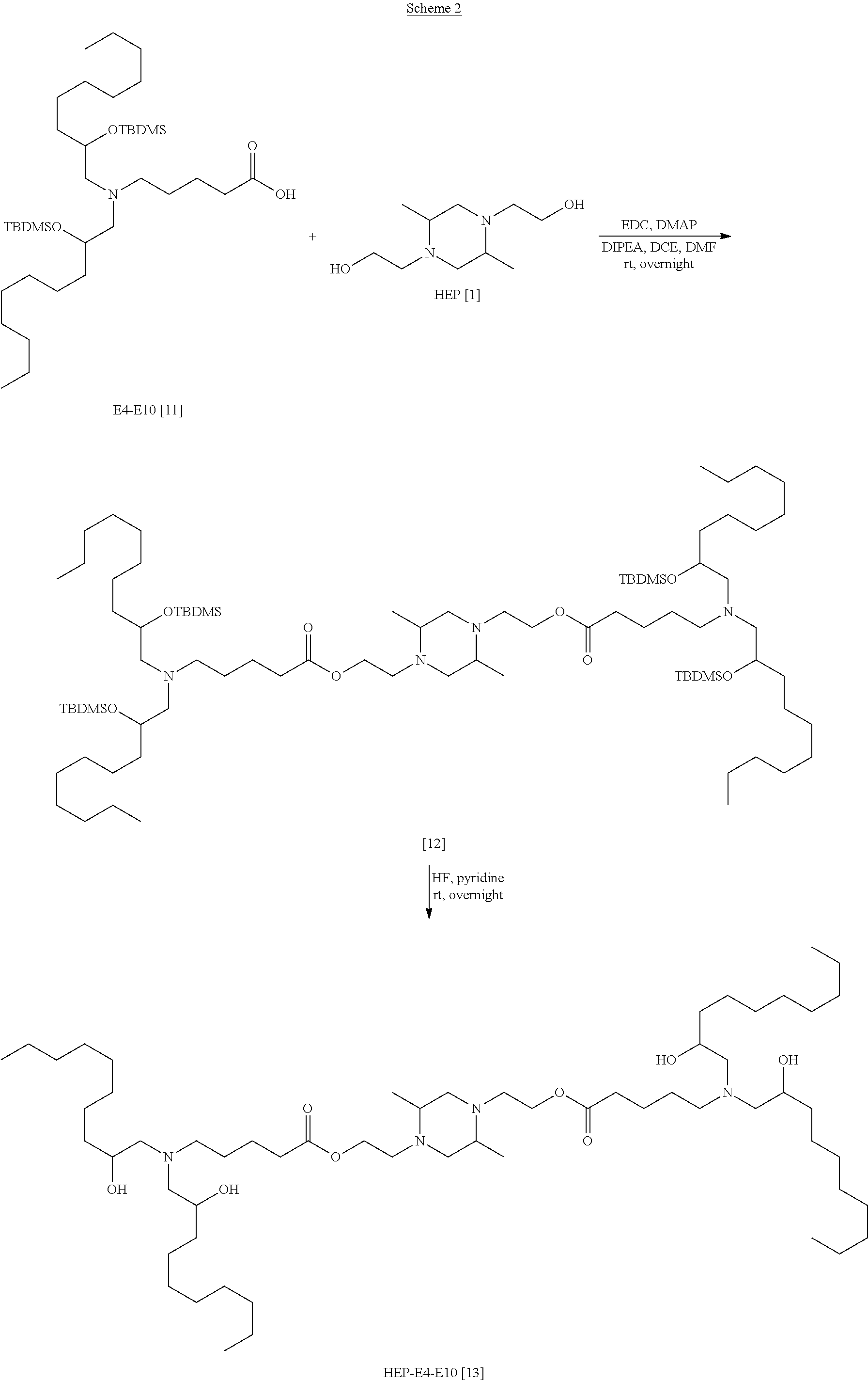 [SMILES: CCCCCCCCC(CN(CCCCC(=O)O)CC(CCCCCCCC)O[Si](C)(C)C(C)(C)C)O[Si](C)(C)C(C)(C)C ; CC1CN(CCO)C(C)CN1CCO ; CCCCCCCCC(CN(CCCCC(=O)OCCN1CC(C)N(CCOC(=O)CCCCN(CC(CCCCCCCC)O[Si](C)(C)C(C)(C)C)CC(CCCCCCCC)O[Si](C)(C)C(C)(C)C)CC1C)CC(CCCCCCCC)O[Si](C)(C)C(C)(C)C)O[Si](C)(C)C(C)(C)C ; CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

### Synthetic Protocol

### Synthesis of [12]

As set out in Scheme 2: To a solution of HEP [1](0.100 g, 0.494 mmol, 1.0 eq), E4-E10 [11](0.683 g, 1.038 mmol, 2.1 eq), 1 ml of dimethylformamide, 3 ml of dichloroethane, diisopropylethylamine (0.344 μL, 1.98 mmol, 4.0 eq), and N,N-Dimethylaminopyridine (0.024 g, 0.198 mmol, 0.4 eq) was added 1-Ethyl-3-(3-dimethylaminopropyl)carbodiimide (0.285 g, 1.48 mmol, 3.0 eq) and allowed to react at room temperature overnight (18 hr). Afterwards, the reaction mixture was concentrated using a rotavapor and purified using a Buchi Combi-flash system on 12 g, 40 μm-sized silica gel columns using hexanes/ethyl acetate as the mobile phase, yielding a colorless oil (63.3% yield).

### Synthesis of HEP-E4-E10 [13]

As set out in Scheme 2: To a 20 ml Polypropylene scintillation vial equipped with a PTFE stir-bar was added 1121 (0.450 g, 0.303 mmol, 1.0 eq) along with 4 ml of dry tetrahydrofuran. The vial was cooled to 0-5° C. on an ice bath and HF/pyridine (1.55 ml, 59.920 mmol, 197.3 eq) was added dropwise. After addition, the reaction vial was allowed to warm to room temperature and stirred overnight (18 hr). Afterwards, the reaction mixture was neutralized with saturated sodium bicarbonate at 0° C. Ethyl acetate was used for extraction (3×). The organic layers were combined, washed with saturated sodium chloride (4×), dried with sodium sulfate, filtered, and rotovaped to yield an off-yellow oil. This oil was further purified using a Buchi Combi-flash system on 12 g, 40 μm-sized silica gel columns using dichloromethane/methanol (3%) as the mobile phase, yielding a colorless oil (48.4% yield).

1H NMR (400 MHz, CDCl3) 4.16 (t, 4H), 3.62 (br, 4H), 2.96 (q, 3H), 2.76 (d. 4H), 2.56 (m, 8H), 2.40 (m, 4H), 2.32 (t, 4H), 2.13 (t, 2H), 1.61 (m, 4H), 1.46 (m, 8H), 1.37 (m, 8H), 1.28 (br, 44H), 1.03 (d, 6H), 0.87 (t, 12H).

13C NMR (400 MHz, CDCl3) 173.65 (2C), 69.65 (2C), 68.04 (2C), 62.84 (2C), 61.82 (2C), 61.44 (2C), 60.89 (2C), 55.57 (4C), 51.55 (2C), 35.35 (4C), 34.20 (2C), 32.09 (7C), 30.00 (5C), 29.77 (6C), 29.47 (6C), 26.93 (2C), 25.84 (5C), 22.84 (9C), 17.77 (2C), 14.30 (7C).

Expected M/Z=1025.64, Observed=1025.8.

### Example 10. Evaluating Cationic Lipids for Pulmonary Delivery

This example demonstrates that a structurally diverse group of cationic lipids are effective in inducing expression of a protein that is encoded by an mRNA encapsulated in lipid nanoparticles prepared with these cationic lipids.

In this example, various cationic lipids were tested for in Wvo efficacy when mRNA encapsulated in lipid nanoparticles (mRNA-LNP) were administered to mice by pulmonary delivery. The cationic lipids were tested for both potency, as determined by levels of protein production, and tolerability, as determined by side effects associated with clearance and metabolism.

About 150 cationic lipids were tested. (FIG. 16). Each cationic lipid was used in preparing lipid nanoparticles encapsulating mRNA encoding firefly luciferase protein (FFL mRNA) according to methods known in the art. For example, suitable methods for mRNA encapsulation include methods described in International Publication Nos. WO2016/004318 and WO 2018/089801, which are hereby incorporated by reference in their entirety. The tested lipid nanoparticles comprised a lipid component consisting of a cationic lipid, a non-cationic lipid (DOPE), a PEG-modified lipid (DMG-PEG2K), and optionally cholesterol.

Lipid nanoparticle formulations comprising FFL mRNA were administered to mice. At approximately 5 hours post-dose, the animals were dosed with luciferin by intraperitoneal injection and all animals were imaged using an IVIS imaging system to measure luciferase production in the lung. FIG. 16 shows that each cationic lipid has various efficacy of in vivo protein expression in the lung. Some cationic lipids remarkably had greater than 50-fold increase in pulmonary protein expression as compared to other cationic lipids.

Based on their performance in this in vivo screen, eight cationic lipids were selected for further investigation in lipid nanoparticles with a lipid component consisting of a cationic lipid, a non-cationic lipid, a PEG-modified lipid, and cholesterol or a cholesterol analogue: GL-TES-SA-DME-E18-2, TL1-01D-DMA, SY-3-E14-DMAPr, TL1-1 OD-DMA, GL-TES-SA-DMP-E18-2, HEP-E4-E10, HEP-E3-E10, and TL1-04D-DMA. Of these, HEP-E4-E10, HEP-E3-E10, GL-TES-SA-DME-E18-2, GL-TES-SA-DMP-E18-2, TL1-01D-DMA and TL1-04D-DMA displayed particularly high potency as determined by the average radiance detected in mouse lungs.

This example shows that various cationic lipids can usefully be employed to prepare lipid nanoparticles with a lipid component consisting of a cationic lipid, a non-cationic lipid, a PEG-modified lipid, and cholesterol or a cholesterol analogue. These lipid nanoparticles can be used effectively to encapsulate mRNA and deliver it to the lungs of subjects to induce expression of the protein encoded by the mRNA.

### Example 11. Excipient Optimization

This example demonstrates that optimization of the lipid nanoparticle formulation can reduce the amount of additional excipients that are required in order to improve nebulization characteristics or maintain the size and encapsulation efficiency of the lipid nanoparticles during lyophilization.

The preceding experiments demonstrate that the nebulization properties and in vivo potency of an mRNA-encapsulating lipid nanoparticle can be improved by adjusting the total lipid:mRNA ratio (mg:mg) to 19:1 or less. This can be achieved by increasing the molar ratio of the cationic lipid to greater than 40% (molar ratio) and reducing the molar ratio of the non-cationic lipid. To determine how the change in total lipid content and the associated change in the sugar to lipid ratio would affect the size of the lipid nanoparticles before and after lyophilization, standard and optimized lipid nanoparticles were suspended in an aqueous solution comprising either trehalose or sucrose at either 8% or 10% (w/v). The composition of the lipid nanoparticles are shown in Table 6A. The sugar to lipid ratios are shown in Table 6B.

TABLE 6A

| DMG- | SY-3-E14- | | | Lipid amount (mg) |
|---|---|---|---|---|
| PEG2K | DMAPr | Chol | DOPE | per 1 mg mRNA |
| 5 | 40 | 25 | 30 | 24 |
| 5 | 50 | 30 | 15 | 19 |
| 5 | 60 | 25 | 10 | 16 |

TABLE 6B

| Sugar % | 30% DOPE | 15% DOPE | 10% DOPE |
|---|---|---|---|
| 10% | 7 | 9 | 10 |
| 8% | 6 | 7 | 8 |

The size and encapsulation efficiency of the lipid nanoparticle formulations was determined before and after lyophilization. The size and encapsulation efficiency after lyophilisation was determined by reconstituting the lyophilized compositions in water. As can be seen from FIG. 17A, a standard lipid formulation comprising 40% cationic lipid and 30% cationic lipid (molar ratios) dramatically changed in size post-lyophilization when suspended in either 10% or 8% trehalose. Moreover, as can be seen in FIG. 17B, the size change was accompanied by a loss of 50% or more in encapsulation efficiency. In contrast, such changes in size were not seen, when sucrose was used.

Surprisingly, the two optimized lipid nanoparticle formulations were able to maintain their size and encapsulation efficiency post-lyophilization, independent of whether trehalose or sucrose was used as an excipient. Reconstituted lipid nanoparticles with a total lipid:mRNA ratio of 19:1 or less maintained a size of less than 150 nm after lyophilization (see FIGS. 17C and 17E). Moreover, the encapsulation efficiency remain above 90% (see FIGS. 17D and 17F).

For the tested lipid nanoparticle formulations, sucrose was more effective than trehalose in maintaining the size of the lipid nanoparticles during lyophilization (cf. FIGS. 17A, 17C and 17E). This was the case even when the sugar-to-lipid ratio was reduced by lowering the disaccharide concentration from 10% to 8% (cf. Table 6B). Therefore, sucrose at a concentration of 8% (w/v) was selected for the optimized lipid nanoparticle formulations.

Next, the nebulization characteristics of optimized lipid nanoparticle formulation was compared to previous lipid nanoparticle formulations. Previous lipid nanoparticle formulations optimized for nebulization contained trehalose as an excipient. Moreover, inclusion of the surfactant D-α-tocopheryl polyethylene glycol 1000 succinate (TPGS) in such trehalose-based formulations was shown to improve nebulization output rates. These optimization steps have been described in U.S. Provisional Patent Application No. 63/217,633, filed on 1 Jul. 2021, which is incorporated herewith by reference) and employed standard lipid formulations comprising DMG-PEG2k:cationic lipid:Chol:DOPE at molar ratios of 5:40:25:30.

Standard lipid nanoparticle formulations comprising SY-3-E14-DMAPr as the cationic lipid component and DOPE as the non-cationic lipid component and encapsulating two different mRNAs were compared to an optimized lipid nanoparticle formulation identified in Example 5. The lipid nanoparticles with the standard formulation included 0.5% TPGS in addition to sucrose to improve the nebulization output rate. The optimized lipid nanoparticle formulation included sucrose as the only excipient.

As can be seen from Table 6C below, the optimized lipid nanoparticle formulation maintained a post-nebulization encapsulation efficiency of close to 90% although the total mRNA:lipid ratio was reduced. Moreover, the nebulization output rate of the optimized formulation was comparable to the nebulization output rate of the standard formations, despite the absence of TPGS.

TABLE 6C

| Test formulation | Standard | | Optimized | |
|---|---|---|---|---|
| Lipid composition | 5:40:25:30 | | 5:50:30:15 | |
| (DMG-PEG2k: | | | | |
| SY-3-E14- | | | | |
| DMAPr:Chol:DOPE) | | | | |
| Encapsulated nucleic | mRNA 1 | mRNA 2 | mRNA1 | mRNA2 |
| acid | | | | |
| Pre-Nebulization | 93 | 94 | 97 | 96 |
| Encapsulation Efficiency | | | | |
| (target: >80%) | | | | |
| Pre-Nebulization Size | 67 | 65 | 53 | 55 |
| (nm) (target: <150 nm) | | | | |
| Pre-Nebulization PDI | 0.194 | 0.21 | 0.177 | 0.185 |
| (target: PDI <0.25) | | | | |
| Nebulization Condition | 0.6 mg/ml, 8% | | 0.6 mg/ml, | |
| | trehalose, 0.5% | | 8% sucrose | |
| | TPGS (w/v) | | | |
| Nebulization rates | 14 | 13 | 14 | 14 |
| (target: 12-15 ml/h) | | | | |
| Post-Nebulization EE % | 75 (19%) | 77 (18%) | 93 (5%) | 88 (10%) |
| (target: change no greater | | | | |
| than 20%) | | | | |

Changes in the salt and buffer concentrations can also affect how effective lipid nanoparticle formulations are nebulized. For example, optimizing the salt and buffer concentrations can reduce the amount of trehalose needed to maintain favourable nebulization characteristics (see U.S. Provisional Patent Application No. 63/217,633). Standard lipid nanoparticle formulations comprising TL1-01D-DMA as the cationic lipid component and DOPE as the non-cationic lipid component and encapsulating two different mRNAs were compared to an optimized lipid nanoparticle formulation identified in Example 7. These standard lipid nanoparticles were formulated with 4% trehalose, 10 mM phosphate buffer (pH 5) and 150 mM NaCl to improve the nebulization output rate.

As can be seen from Table 6D below, the optimized lipid nanoparticle formulation comprising sucrose in place of trehalose achieved nebulization output rates comparable to those achieved with the standard formulations at 75% reduced buffer concentration, while maintaining a post-nebulization encapsulation efficiency of about 90%.

TABLE 6D

| Test formulation | Standard | | Optimized | |
|---|---|---|---|---|
| Lipid composition | 3:40:25:32 | | 3:47:27.5:22.5 | |
| (DMG-PEG2k:TL1- | | | | |
| 01D- | | | | |
| DMA:Chol:DOPE) | | | | |
| Encapsulated nucleic | mRNA 1 | mRNA 2 | mRNA1 | mRNA2 |
| acid | | | | |
| Pre-Nebulization | 96 | 92 | 93 | 91 |
| Encapsulation | | | | |
| Efficiency | | | | |
| (target: >80%) | | | | |
| Pre-Nebulization Size | 64 | 63 | 46 | 52 |
| (nm) (target: <150 nm) | | | | |
| Pre-Nebulization PDI | 0.152 | 0.173 | 0.143 | 0.16 |
| (target: PDI <0.25) | | | | |
| Nebulization Condition | 0.4 mg/ml, 4% | | 0.4 mg/ml, 4% | |
| | trehalose, 10 mM | | sucrose, 2.5 mM | |
| | phosphate buffer | | phosphate buffer | |
| | (pH 5), | | (pH 5.5), | |
| | 150 mM NaCl | | 150 mM NaCl | |
| Nebulization rates | 12 | 14 | 13 | 13 |
| (target: 12-15 ml/h) | | | | |
| Post-Nebulization | 88 (8%) | 90 (2%) | 88 (10%) | 90 (9%) |
| EE % (target: change | | | | |
| no greater than 20%) | | | | |

This example shows that optimization of the lipid composition can reduce the requirements on excipients in a lipid nanoparticle formulation that may otherwise be needed to improve the nebulization characteristics of the formulation or to maintain the size and encapsulation efficiency of the lipid nanoparticles during lyophilization.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A lipid nanoparticle comprising
(i) an mRNA encapsulated within the lipid nanoparticle, and
(ii) a lipid component consisting of the following components:
a. a cationic lipid component,
b. a non-cationic lipid component,
c. a PEG-modified lipid component, and
d. cholesterol component
wherein:
(1) the cationic lipid component is greater than 40% (molar ratio);
(2) the non-cationic lipid component is less than 25% (molar ratio); and
(3) a total lipid:mRNA ratio (mg:mg) is 19:1 or less.

2. The lipid nanoparticle of claim 1, wherein the total lipid:mRNA ratio (mg:mg) is between 11:1 and 19:1.

3. The lipid nanoparticle of claim 1 or claim 2, wherein the cationic lipid component is 45%-60% (molar ratio).

4. The lipid nanoparticle of claim 3, wherein the cationic lipid component is 45%-55% (molar ratio).

5. The lipid nanoparticle of claim 4, wherein the cationic lipid component is about 50% (molar ratio).

6. The lipid nanoparticle of any preceding claim, wherein the non-cationic lipid component is about 22.5% (molar ratio), or less.

7. The lipid nanoparticle of claim 6, wherein the non-cationic lipid component is less than 18% (molar ratio).

8. The lipid nanoparticle of claim 7, wherein the non-cationic lipid component is about 15% (molar ratio), or less.

9. The lipid nanoparticle of claim 8, wherein the non-cationic lipid component is less than 13% (molar ratio).

10. The lipid nanoparticle of any preceding claim, wherein cholesterol component is cholesterol or a cholesterol analogue.

11. The lipid nanoparticle of any preceding claim, wherein the molar ratios of the lipid components are:
a. about 47%-60% cationic lipid,
b. about 10%-22.5% non-cationic lipid,
c. about 3%-5% PEG-modified lipid, and
d. the remainder is cholesterol or a cholesterol analogue.

12. The lipid nanoparticle of claim 11, wherein the molar ratios of the lipid components are:
a. about 50%-55% cationic lipid,
b. about 10-15% non-cationic lipid,
c. about 3-5% PEG-modified lipid, and
d. the remainder is cholesterol or cholesterol analogue.

13. The lipid nanoparticle of claim 11 or 12, wherein the molar ratios of the lipid components are:
a. about 55% cationic lipid,
b. about 10% non-cationic lipid,
c. about 5% PEG-modified lipid, and
d. about 30% cholesterol or cholesterol analogue.

14. The lipid nanoparticle of claim 11 or 12, wherein the molar ratios of the lipid components are:
a. about 50% cationic lipid,
b. about 12.5% non-cationic lipid,
c. about 5% PEG-modified lipid, and
d. about 32.5% cholesterol or cholesterol analogue.

15. The lipid nanoparticle of claim 11 or 12, wherein the molar ratios of the lipid components are:
a. about 50% cationic lipid,
b. about 15% non-cationic lipid,
c. about 5% PEG-modified lipid, and
d. about 30% cholesterol or cholesterol analogue.

16. The lipid nanoparticle of claim 11, wherein the molar ratios of the lipid components are:
a. about 47% cationic lipid,
b. about 22.5% non-cationic lipid,
c. about 3% PEG-modified lipid, and
d. about 27.5% cholesterol or cholesterol analogue.

17. The lipid nanoparticle of any one of the preceding claims, wherein the cationic lipid is SY-3-E14-DMAPr.

18. The lipid nanoparticle of any one of the preceding claims, wherein the cationic lipid is TL1-01D-DMA.

19. The lipid nanoparticle of any preceding claim, wherein the lipid nanoparticle is any one of the lipid nanoparticles in Tables A, B, C, D, E, F, G and H.

20. The lipid nanoparticle of any preceding claim, wherein the total lipid:mRNA ratio (mg:mg) is about 18:1 or less.

21. The lipid nanoparticle of claim 20, wherein the total lipid:mRNA ratio (mg:mg) is about 17:1 or less.

22. The lipid nanoparticle of claim 21, wherein the total lipid:mRNA ratio (mg:mg) is about 15:1 or less.

23. The lipid nanoparticle of any preceding claim, wherein the lipid nanoparticle is capable of being nebulized at a nebulization output rate of greater than about 12 ml/h.

24. The lipid nanoparticle of claim 23, wherein the lipid nanoparticle is capable of being nebulized at a nebulization output rate of greater than about 15 ml/h, or greater than about 20 ml/h.

25. The lipid nanoparticle of any preceding claim, wherein the encapsulation efficiency of the lipid nanoparticle after nebulization is no more than about 10% lower than the encapsulation efficiency of the lipid nanoparticle before nebulization.

26. The lipid nanoparticle of any preceding claim, wherein the lipid nanoparticle has an encapsulation efficiency before and after nebulization of at least about 90%.

27. A lipid nanoparticle comprising
(iii) an mRNA encapsulated within the lipid nanoparticle, and
(iv) a lipid component consisting of the following lipids with molar ratios of;
a. 41%-70/o of a cationic lipid,
b. 9%-18% of a non-cationic lipid,
c. 2%-6% of a PEG-modified lipid, and
d. 9%-48% of cholesterol or a cholesterol analogue.

28. The lipid nanoparticle of claim 27, wherein the lipid nanoparticle is capable of being nebulized.

29. The lipid nanoparticle of claim 27, wherein the lipid nanoparticle is capable of being nebulized at a nebulization output rate of greater than about 12 ml/h, in particular at a nebulization output rate of greater than about 15 ml/h.

30. The lipid nanoparticle of any of claims 27-29, wherein the molar ratio of the cationic lipid is 45%-70%.

31. The lipid nanoparticle of any of claims 27-30, wherein the molar ratio of the cationic lipid is 45%-65%.

32. The lipid nanoparticle of any of claims 27-31, wherein the molar ratio of the cationic lipid is 50%-70%.

33. The lipid nanoparticle of any of claims 27-32, wherein the molar ratio of the cationic lipid is 50%-65%.

34. The lipid nanoparticle of any of claims 27-33, wherein the molar ratio of the cationic lipid is 50%-60%.

35. The lipid nanoparticle of any of claims 27-34, wherein the molar ratio of the cationic lipid is about 50%.

36. The lipid nanoparticle of any of claims 27-35, wherein the molar ratio of the cationic lipid is about 55%.

37. The lipid nanoparticle of any of claims 27-36, wherein the molar ratio of the cationic lipid is about 60%.

38. The lipid nanoparticle of any of claims 27-37, wherein the molar ratio of the non-cationic lipid is 9%-15%.

39. The lipid nanoparticle of any of claims 27-38, wherein the molar ratio of the non-cationic lipid is 10%-15%.

40. The lipid nanoparticle of any of claims 27-39, wherein the molar ratio of the non-cationic lipid is about 15%.

41. The lipid nanoparticle of any of claims 27-40, wherein the molar ratio of the non-cationic lipid is about 12.5%.

42. The lipid nanoparticle of any of claims 27-41, wherein the molar ratio of the non-cationic lipid is about 10%.

43. The lipid nanoparticle of any of claims 27-42, wherein the molar ratio of the PEG-modified lipid is 3%-6%.

44. The lipid nanoparticle of any of claims 27-43, wherein the molar ratio of the PEG-modified lipid is 4%-6%.

45. The lipid nanoparticle of any of claims 27-44, wherein the molar ratio of the PEG-modified lipid is about 5%.

46. The lipid nanoparticle of any of claims 27-45, wherein the molar ratio of the PEG-modified lipid is about 3%.

47. The lipid nanoparticle of any of claims 27-46, wherein the molar ratio of the cholesterol or cholesterol analogue is 10%-45%.

48. The lipid nanoparticle of any of claims 27-47, wherein the molar ratio of the cholesterol or cholesterol analogue is 10%-30%.

49. The lipid nanoparticle of any of claims 27-48, wherein the molar ratio of the cholesterol or cholesterol analogue is 25%-30%.

50. The lipid nanoparticle of any of claims 27-49, wherein the molar ratio of the cholesterol or cholesterol analogue is about 25%.

51. The lipid nanoparticle of any of claims 27-50, wherein the molar ratio of the cholesterol or cholesterol analogue is about 30%.

52. The lipid nanoparticle of any preceding claim, wherein the molar ratios of the lipids/lipid components are:
a. 50%-60% cationic lipid,
b. 9%-18% non-cationic lipid,
c. 4%-6% PEG-modified lipid, and
d. 20-35% cholesterol or cholesterol analogue.

53. The lipid nanoparticle of any preceding claim, wherein the molar ratios of the lipids/lipid components are:
a. 50%-60% cationic lipid,
b. 9%-15% non-cationic lipid,
c. 4%-6% PEG-modified lipid, and
d. 25-30% cholesterol or cholesterol analogue.

54. The lipid nanoparticle of any preceding claim, wherein the molar ratios of the lipids/lipid components are:
a. about 50% cationic lipid,
b. about 15% non-cationic lipid,
c. about 5% PEG-modified lipid, and
d. about 30% cholesterol or cholesterol analogue.

55. The lipid nanoparticle of any preceding claim, wherein the molar ratios of the lipids/lipid components are:
a. about 60% cationic lipid,
b. about 10% non-cationic lipid,
c. about 5% PEG-modified lipid, and
d. about 25% cholesterol or cholesterol analogue.

56. The lipid nanoparticle of any preceding claim, wherein the molar ratios of the lipids/lipid components are:
a. about 50% cationic lipid,
b. about 10% non-cationic lipid,
c. about 5% PEG-modified lipid, and
d. about 35% cholesterol or cholesterol analogue.

57. The lipid nanoparticle of any preceding claim, wherein the molar ratios of the lipids/lipid components are:
a. about 50% cationic lipid,
b. about 12.5% non-cationic lipid,
c. about 5% PEG-modified lipid, and
d. about 32.5% cholesterol or cholesterol analogue.

58. The lipid nanoparticle of any preceding claim, wherein the molar ratios of the lipids/lipid components are:
a. about 50% cationic lipid,
b. about 17.5% non-cationic lipid,
c. about 5% PEG-modified lipid, and
d. about 27.5% cholesterol or cholesterol analogue.

59. The lipid nanoparticle of any preceding claim, wherein the molar ratios of the lipids/lipid components are:
a. about 55% cationic lipid,
b. about 10% non-cationic lipid,
c. about 5% PEG-modified lipid, and
d. about 30% cholesterol or cholesterol analogue.

60. The lipid nanoparticle of any preceding claim, wherein the molar ratios of the lipids/lipid components are:
a. about 55% cationic lipid,
b. about 12.5% non-cationic lipid,
c. about 5% PEG-modified lipid, and
d. about 27.5% cholesterol or cholesterol analogue.

61. The lipid nanoparticle of any preceding claim, wherein the molar ratios of the lipids/lipid components are:
a. about 55% cationic lipid,
b. about 15% non-cationic lipid,
c. about 5% PEG-modified lipid, and
d. about 25% cholesterol or cholesterol analogue.

62. The lipid nanoparticle of any preceding claim, wherein the molar ratios of the lipids/lipid components are:
a. about 55% cationic lipid,
b. about 17.5% non-cationic lipid,
c. about 5% PEG-modified lipid, and
d. about 22.5% cholesterol or cholesterol analogue.

63. The lipid nanoparticle of any preceding claim, wherein the molar ratios of the lipids/lipid components are:
a. about 60% cationic lipid,
b. about 12.5% non-cationic lipid,
c. about 5% PEG-modified lipid, and
d. about 22.5% cholesterol or cholesterol analogue.

64. The lipid nanoparticle of any preceding claim, wherein the molar ratios of the lipids/lipid components are:
a. about 60% cationic lipid,
b. about 15% non-cationic lipid,
c. about 5% PEG-modified lipid, and
d. about 20% cholesterol or cholesterol analogue.

65. The lipid nanoparticle of any preceding claim, wherein the lipid nanoparticle is any one of the lipid nanoparticles in Tables A, B, C, D, E, F, or G.

66. The lipid nanoparticle of any preceding claim, wherein the lipid nanoparticle has an encapsulation efficiency before and after nebulization of at least about 90%.

67. The lipid nanoparticle of any preceding claim, wherein the lipid nanoparticle has an encapsulation efficiency before and after nebulization of at least about 95%.

68. The lipid nanoparticle of any preceding claim, wherein the lipid nanoparticle has an encapsulation efficiency before and after nebulization of at least about 96%.

69. The lipid nanoparticle of any preceding claim, wherein the lipid nanoparticle has an encapsulation efficiency before and after nebulization of at least about 97%.

70. The lipid nanoparticle of any preceding claim, wherein the lipid nanoparticle has an encapsulation efficiency before and after nebulization of at least about 98%.

71. The lipid nanoparticle of any preceding claim, wherein the lipid nanoparticle has an encapsulation efficiency before and after nebulization of at least about 99%.

72. The lipid nanoparticle of any preceding claim, wherein the encapsulation efficiency of the lipid nanoparticle after nebulization is no more than about 20% lower than the encapsulation efficiency of the lipid nanoparticle before nebulization.

73. The lipid nanoparticle of any preceding claim, wherein the encapsulation efficiency of the lipid nanoparticle after nebulization is no more than about 15% lower than the encapsulation efficiency of the lipid nanoparticle before nebulization.

74. The lipid nanoparticle of any preceding claim, wherein the encapsulation efficiency of the lipid nanoparticle after nebulization is no more than about 10% lower than the encapsulation efficiency of the lipid nanoparticle before nebulization.

75. The lipid nanoparticle of any preceding claim, wherein the encapsulation efficiency of the lipid nanoparticle after nebulization is no more than about 5% lower than the encapsulation efficiency of the lipid nanoparticle before nebulization.

76. The lipid nanoparticle of any preceding claim, wherein the encapsulation efficiency of the lipid nanoparticle after nebulization is no more than about 3% lower than the encapsulation efficiency of the lipid nanoparticle before nebulization.

77. The lipid nanoparticle of any preceding claim, wherein the encapsulation efficiency of the lipid nanoparticle after nebulization is about the same as the encapsulation efficiency of the lipid nanoparticle before nebulization.

78. The lipid nanoparticle of any preceding claim, wherein the lipid nanoparticle is for pulmonary delivery by nebulization.

79. The lipid nanoparticle of any one of claims 66-78, wherein the nebulization is performed with a nebulizer comprising vibrating mesh technology (VMT).

80. The lipid nanoparticle of any preceding claim, wherein the cationic lipid has a structure according to Formula (IIA): 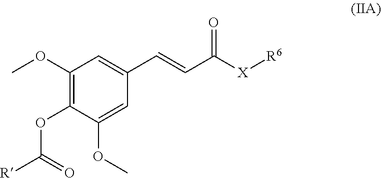
wherein X is O or S;
wherein R′ is 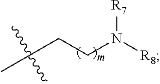
wherein R⁶ is 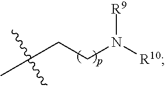
wherein m and p are each independently 0, 1, 2, 3, 4 or 5;
wherein R⁷ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₖR^{A} or —(CH₂)ₖCH(OR¹¹)R^{A};
wherein R⁹ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₙR^{B} or —(CH₂)ₙCH(OR¹²)R^{B};
wherein R⁹ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)_{q}R^{C} or —(CH₂)_{q}CH(OR¹³)R^{C};
wherein R¹⁰ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ᵣR^{D} or —(CH₂)ᵣCH(OR⁴)R^{D};
wherein k, n, q and r are each independently 1, 2, 3, 4, or 5;
or wherein (i) R⁷ and R⁸ or (ii) R⁹ and R¹⁰ together form an optionally substituted 5- or 6-membered heterocycloalkyl or heteroaryl wherein the heterocycloalkyl or heteroaryl comprises 1 to 3 heteroatoms selected from N, O and S;
wherein R¹¹, R¹², R¹³ and R¹⁴ are each independently selected from H, methyl, ethyl or propyl
wherein R^{A}, R^{B}, R^{C} and R^{D} are each independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)alkyl, optionally substituted —OC(O)alkenyl, optionally substituted (C₁-C₆) monoalkylamino, optionally substituted (C₁-C₆) dialkylamino, optionally substituted (C₁-C₆)alkoxy, —OH, —NH—;
wherein at least one of R⁷, R⁸, R⁹, R¹⁰ comprises a R^{A}, R^{B}, R^{C} or R^{D} moiety respectively wherein that R^{A}, R^{B}, R^{C} or R^{D} is independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)C₆-C₂₀)alkyl or optionally substituted —OC(O)(C₆-C₂₀)alkenyl;
or a pharmaceutically acceptable salt thereof.

81. The lipid nanoparticle of any one of claims 1-79, wherein the cationic lipid has a structure according to Formula (IIID): 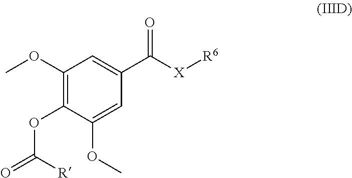
wherein X is O or S,
wherein R′ is 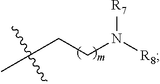
wherein R⁶ is 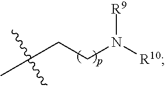
wherein m and p are each independently 0, 1, 2, 3, 4 or 5;
wherein R⁷ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₖR^{A} or —(CH₂)CH(OR¹¹)R^{A};
wherein R⁸ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ₙR^{B} or —(CH₂)ₙCH(OR¹²)R^{B};
wherein R⁹ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)_{q}R^{C} or —(CH₂)_{q}CH(OR¹³)R^{C};
wherein R¹⁰ is selected from H, optionally substituted (C₁-C₆)alkyl, optionally substituted (C₂-C₆)alkenyl, optionally substituted (C₂-C₆)alkynyl, optionally substituted (C₁-C₆)acyl, —(CH₂)ᵣR^{D} or —(CH₂)ᵣCH(OR⁴)R^{D};
wherein k, n, q and r are each independently 1, 2, 3, 4, or 5;
or wherein (i) R⁷ and R⁸ or (ii) R⁹ and R¹⁰ together form an optionally substituted 5- or 6-membered heterocycloalkyl or heteroaryl wherein the heterocycloalkyl or heteroaryl comprises 1 to 3 heteroatoms selected from N, O and S;
wherein R¹¹, R¹², R¹³ and R¹⁴ are each independently selected from H, methyl, ethyl or propyl
wherein R^{A}, R^{B}, R^{C} and R^{D} are each independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)alkyl, optionally substituted —OC(O)alkenyl, optionally substituted (C₁-C₆) monoalkylamino, optionally substituted (C₁-C₆) dialkylamino, optionally substituted (C₁-C₆)alkoxy, —OH, —NH—;
wherein at least one of R⁷, R⁸, R⁹, R¹⁰ comprises a R^{A}, R^{B}, R^{C} or R^{D} moiety respectively wherein that R^{A}, R^{B}, R^{C} or R^{D} is independently selected from optionally substituted (C₆-C₂₀)alkyl, optionally substituted (C₆-C₂₀)alkenyl, optionally substituted (C₆-C₂₀)alkynyl, optionally substituted (C₆-C₂₀)acyl, optionally substituted —OC(O)C₆-C₂₀)alkyl or optionally substituted —OC(O)(C₆-C₂₀)alkenyl;
or a pharmaceutically acceptable salt thereof.

82. The lipid nanoparticle of claim 80 or claim 81, wherein X is O.

83. The lipid nanoparticle of any one of claims 80-82, wherein m is 1, 2 or 3.

84. The lipid nanoparticle of any one of claims 80-83, wherein p is 1, 2 or 3.

85. The lipid nanoparticle of any one of claims 80-84, wherein R′ is;

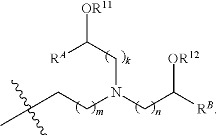

86. The lipid nanoparticle of claim 85 wherein:
i) k, m and n=1; or
ii) k, m and n=1 and R¹¹ and R²═H; or
iii) k and n=1, and m=2; or
iv) k and n=1, m=2 and R¹¹ and R¹²═H; or
v) k and n=1, and m=3; or
vi) k and n=1, m=3 and R¹¹ and R¹²═H.

87. The lipid nanoparticle of any one of claims 80-86, wherein

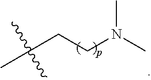

88. The lipid nanoparticle of any one of claims 80-87, wherein R⁶ is

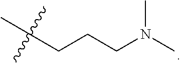 [SMILES: *CCCN(C)C]

89. The lipid nanoparticle of any one of claims 80-86, wherein R⁶ is selected from the group consisting of:

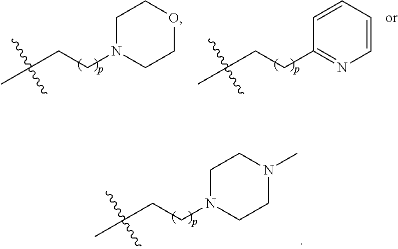

90. The lipid nanoparticle of any one of claims 80-86, wherein R⁶ is selected from the group consisting of:

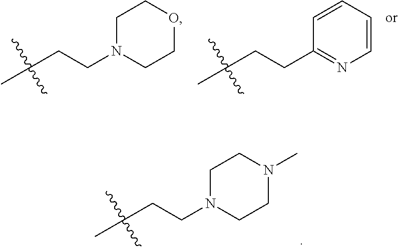

91. The lipid nanoparticle of any one of claims 80-88, wherein R⁶ is 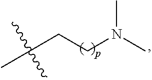 [SMILES: *CCN(C)C]
R′ is 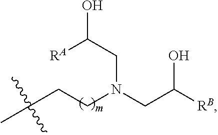
m is 2 and p is 2.

92. The lipid nanoparticle of any one of claims 80-88, wherein R⁶ is 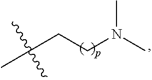 [SMILES: *CCN(C)C]
R′ is 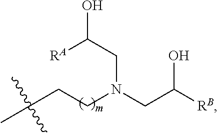
m is 3 and p is 2.

93. The lipid nanoparticle of any one of claims 80-92, wherein R^{A} and R^{B} are each independently selected from optionally substituted (C₆-C₂₀)alkyl, optionally substituted (C₆-C₂₀)alkenyl, optionally substituted (C₆-C₂₀)alkynyl.

94. The lipid nanoparticle of any one of claims 80-92, wherein R^{A} and R^{B} are the same and selected from optionally substituted (C₆-C₂₀)alkyl, optionally substituted (C₆-C₂₀)alkenyl, optionally substituted (C₆-C₂₀)alkynyl.

95. The lipid nanoparticle of any one of claims 80-92, wherein R^{A} and R^{B} are each independently optionally substituted (C₆-C₂₀)alkyl.

96. The lipid nanoparticle of any one of claims 80-92, wherein R^{A} and R^{B} are the same and are optionally substituted (C₆-C₂₀)alkyl.

97. The lipid nanoparticle of any one of claims 80-92, wherein R^{A} and R^{B} are each independently optionally substituted (C₆-C₂₀)alkenyl.

98. The lipid nanoparticle of any one of claims 80-92, wherein R^{A} and R^{B} are the same and are optionally substituted (C₆-C₂₀)alkenyl.

99. The lipid nanoparticle of any one of claims 80-92, wherein R^{A} and R^{B} are each independently optionally substituted (C₆-C₂₀)alkynyl.

100. The lipid nanoparticle of any one of claims 80-92, wherein R^{A} and R^{B} are the same and are optionally substituted (C₁-C₂₀)alkynyl.

101. The lipid nanoparticle of any one of claims 80-92, wherein R^{A} and R^{B} are each independently optionally substituted (C₆-C₂₀)acyl.

102. The lipid nanoparticle of any one of claims 80-92, wherein R^{A} and R^{B} are the same and are optionally substituted (C₆-C₂₀)acyl.

103. The lipid nanoparticle of any one of claims 80-92, wherein R^{A} and R^{B} are each independently optionally substituted —OC(O)C₆-C₂₀)alkyl.

104. The lipid nanoparticle of any one of claims 80-92, wherein R^{A} and R^{B} are the same and are optionally substituted —OC(O)(C₆-C₂₀)alkyl.

105. The lipid nanoparticle of any one of claims 80-92, wherein R^{A} and R^{B} are each independently optionally substituted —OC(O)C₆-C₂₀)alkenyl.

106. The lipid nanoparticle of any one of claims 80-92, wherein R^{A} and R^{B} are the same and are optionally substituted —OC(O)(C₆-C₂₀)alkenyl.

107. The lipid nanoparticle of any preceding claim, wherein the cationic lipid has a structure according to Formula (IIIE): 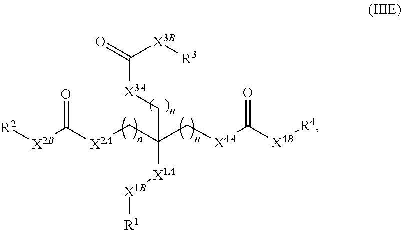
or a pharmaceutically acceptable salt thereof, wherein
each n is independently 0 or 1;
X^{1A} is independently O or NR^{1A};
R^{1A} is H or C₁-C₆ alkyl;
X^{1B} is a covalent bond, C(O), CH₂CO₂, or CH₂C(O);
one of X^{2A} and X^{2B} is O and the other is a covalent bond;
one of X^{3A} and X^{3B} is O and the other is a covalent bond;
one of X^{4A} and X^{4B} is O and the other is a covalent bond;
R¹ is independently L¹-B¹, C₆-C₃₀ alkyl, C₆-C₃₀ alkenyl, or C₆-C₃₀ alkynyl;
R² is independently L²-B², C₆-C₃₀ alkyl, C₆-C₃₀ alkenyl, C₆-C₃₀ alkynyl;
R³ is independently L³-B³, C₆-C₃₀ alkyl, C₆-C₃₀ alkenyl, C₆-C₃₀ alkynyl;
R; is independently L⁴-B⁴, C₆-C₃₀ alkyl, C₆-C₃₀ alkenyl, C₆-C₃₀ alkynyl;
L¹, L², L³, and L⁴ are each independently C₁-C₃₀ alkylene; C₂-C₃v alkenylene; or C₂-C₃₀ alkynylene;
each of B¹, B², B³, and B⁴ is independently an ionizable nitrogen-containing group, and
wherein the cationic lipid comprises at least one ionizable nitrogen-containing group.

108. The lipid nanoparticle of any preceding claim, wherein the cationic lipid has a structure according to Formula (IIIF): 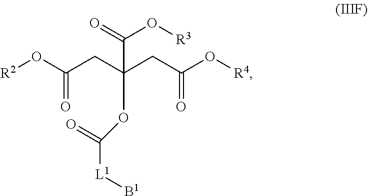
or a pharmaceutically acceptable salt thereof, wherein
B¹ is an ionizable nitrogen-containing group;
L¹ is C₁-C₁₀ alkylene;
each of R², R³, and R⁴ is independently C₆-C₃₀ alkyl, C₆-C₃₀ alkenyl, C₆-C₃₀ alkynyl.

109. The lipid nanoparticle of any preceding claim, wherein the cationic lipid has a structure according to Formula (IIIG): 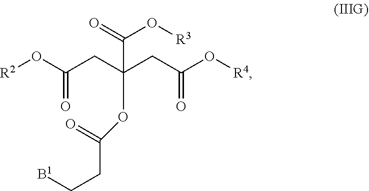
or a pharmaceutically acceptable salt thereof, wherein
B¹ is an ionizable nitrogen-containing group;
each of R², R³, and R is independently C₆-C₃₀ alkyl, C₆-C₃₀ alkenyl, C₆-C₃₀ alkynyl.

110. The lipid nanoparticle of claims 107-109, wherein each of R², R³, and R⁴ is independently C₆-C₁₂ alkyl substituted by —O(CO)R or —C(O)OR, wherein R³ is unsubstituted C₆-C₁₄ alkyl.

111. The lipid nanoparticle of claims 107-109, wherein each of R², R³, and R⁴ is independently:

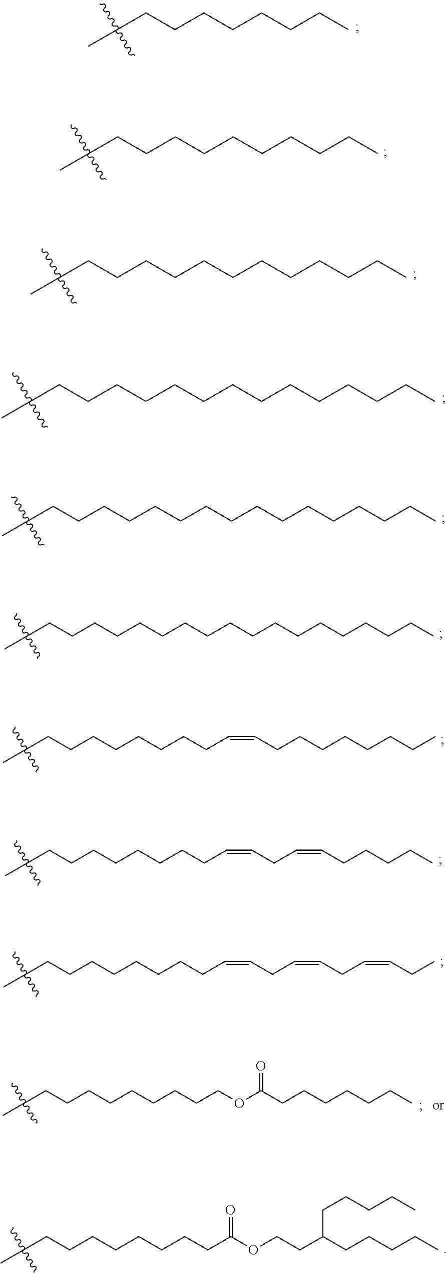

112. The lipid nanoparticle of any of claims 107-111 wherein B¹ is:
g) NH₂, guanidine, amidine, a mono- or dialkylamine, 5- to 6-membered nitrogen-containing heterocycloalkyl, or 5- to 6-membered nitrogen-containing heteroaryl;
h) 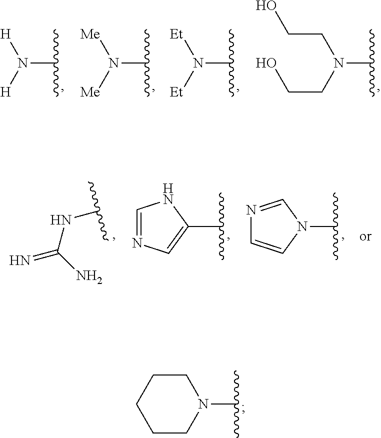
or
i)

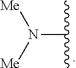 [SMILES: *N(C)C]

113. The lipid nanoparticle of any of claims 107-112, wherein L¹ is C₁-alkylene.

114. The lipid nanoparticle of any preceding claim, wherein the cationic lipid is selected from GL-TES-SA-DMP-E18-2, GL-TES-SA-DME-E18-2, TL1-01D-DMA, TL1-04D-DMA, SY-3-E14-DMAPr, TL1-10D-DMA, HEP-E3-E10, HEP-E4-E10, SI-4-E14-DMAPr, TL1-12D-DMA, SY-010, and SY-011.

115. The lipid nanoparticle of any preceding claim, wherein the cationic lipid is SY-3-E14-DMAPr.

116. The lipid nanoparticle of any preceding claim, wherein the cationic lipid is TL1-01D-DMA.

117. The lipid nanoparticle of any preceding claim, wherein the non-cationic lipid is a PE lipid or a PC lipid.

118. The lipid nanoparticle of any preceding claim, wherein the non-cationic lipid is selected from DOPE, DLoPE, DMPE, DLPE, DOPC, DEPE, DSPC, DPPC, DMPC, DOPC, 16:1PC, and 14:1PC.

119. The lipid nanoparticle of any preceding claim, wherein the non-cationic lipid is a PE lipid.

120. The lipid nanoparticle of claim 119, wherein the non-cationic lipid is DOPE, DLoPE, DMPE, or DLPE.

121. The lipid nanoparticle of any preceding claim, wherein the non-cationic lipid is DOPE.

122. The lipid nanoparticle of any preceding claim, wherein the non-cationic lipid is a PC lipid.

123. The lipid nanoparticle of claim 122, wherein the non-cationic lipid is DOPC, DMPC, DLPC, DPPC or DSPC.

124. The lipid nanoparticle of any preceding claim, wherein the non-cationic lipid is DOPS.

125. The lipid nanoparticle of any preceding claim, wherein the cholesterol or cholesterol analogue is cholesterol.

126. The lipid nanoparticle of any preceding claim, wherein the cholesterol analogue is selected from β-sitosterol, stigmastanol, campesterol, fucosterol, stigmasterol, and dexamethasone.

127. The lipid nanoparticle of any preceding claim, wherein the cholesterol analogue is β-sitosterol.

128. The lipid nanoparticle of any preceding claim, wherein the cholesterol analogue is stigmastanol.

129. The lipid nanoparticle of any preceding claim, wherein the PEG-modified lipid is selected from DMG-PEG2K, 2[(polyethylene glycol)-2000]—N,N-ditetradecylacetamide, and DSPE-PEG2K-COOH.

130. The lipid nanoparticle of any preceding claim, wherein the PEG-modified lipid is DMG-PEG2K.

131. The lipid nanoparticle of any preceding claim, wherein
b. the non-cationic lipid is DOPE,
c. the PEG-modified lipid is DMG-PEG2K, and
d. the cholesterol or cholesterol analogue is cholesterol.

132. The lipid nanoparticle of any preceding claim, wherein
b. the non-cationic lipid is DSPC,
c. the PEG-modified lipid is DMG-PEG2K, and
d. the cholesterol or cholesterol analogue is cholesterol.

133. The lipid nanoparticle of any preceding claim, wherein
a. the cationic lipid is SY-3-E14-DMAPr,
b. the non-cationic lipid is DOPE,
c. the PEG-modified lipid is DMG-PEG2K, and
d. the cholesterol or cholesterol analogue is cholesterol.

134. The lipid nanoparticle of any preceding claim, wherein
a. the cationic lipid is SY-3-E14-DMAPr,
b. the non-cationic lipid is DSPC,
c. the PEG-modified lipid is DMG-PEG2K, and
d. the cholesterol or cholesterol analogue is cholesterol.

135. The lipid nanoparticle of any preceding claim, wherein the lipid nanoparticle has a total lipid:mRNA ratio of less than 19:1 (mg:mg).

136. The lipid nanoparticle of any preceding claim, wherein the lipid nanoparticle is prepared using a total lipid:mRNA ratio of 11:1 to 19:1 (mg:mg).

137. The lipid nanoparticle of any preceding claim, wherein the lipid nanoparticle is prepared using a total lipid:mRNA ratio of about 19:1 (mg:mg).

138. The lipid nanoparticle of any preceding claim, wherein the lipid nanoparticle is prepared using a total lipid:mRNA ratio of about 17:1 (mg:mg).

139. The lipid nanoparticle of any preceding claim, wherein the mRNA encodes a therapeutic protein.

140. The lipid nanoparticle of any preceding claim, wherein the mRNA encodes for cystic fibrosis transmembrane conductance regulator, ATP-binding cassette sub-family A member 3 protein, dynein axonemal intermediate chain 1 (DNAI1) protein, dynein axonemal heavy chain 5 (DNAH5) protein, alpha-1-antitrypsin protein, forkhead box P3 (FOXP3) protein, or one or more surfactant protein.

141. The lipid nanoparticle of any preceding claim, wherein the mRNA is codon-optimized.

142. The lipid nanoparticle of any preceding claim, wherein the mRNA comprises at least one nonstandard nucleobase.

143. The lipid nanoparticle of claim 142, wherein the nonstandard nucleobase is a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N-1-methyl-pseudouridine), 2-thiouridine, and 2-thiocytidine.

144. The lipid nanoparticle of any preceding claim, wherein the lipid nanoparticle has a size less than about 150 nm.

145. The lipid nanoparticle of any preceding claim, wherein the lipid nanoparticle has a size less than about 100 nm.

146. The lipid nanoparticle of any preceding claim, wherein the lipid nanoparticle has a size of 60-150 nm.

147. A composition comprising the lipid nanoparticle of any preceding claim.

148. The composition of claim 147, wherein the composition is formulated for pulmonary delivery by nebulization.

149. The composition of claim 148, wherein the nebulization is performed with a nebulizer comprising vibrating mesh technology (VMT).

150. The composition of any one of claims 147-149, wherein the composition further comprises one or more excipients.

151. The composition of claim 150, wherein the one or more excipients is selected from a buffer, a salt, is a sugar, or combinations thereof.

152. The composition of any one of claims 147-151, wherein the composition further comprises a buffer.

153. The composition of any one of claims 147-152, wherein the composition further comprises a salt.

154. The composition of claim 153, wherein the salt is sodium chloride.

155. The composition of any one of claims 147-154, wherein the excipient is a sugar.

156. The composition of claim 155, wherein the sugar is a disaccharide.

157. The composition of claim 156, wherein the disaccharide is sucrose or trehalose.

158. The composition of claim 156 or claim 157, wherein the disaccharide is at a concentration of about 4% w/v, about 6% w/v, about 8% w/v, or about 10% w/v.

159. The composition of claim 158, wherein the disaccharide is at a concentration of 4%-8% w/v.

160. The composition of claim 159, wherein the disaccharide is sucrose.

161. The composition of claims 157-160, further comprising TPGS at a concentration of about 0.1% w/v to about 1% w/v.

162. The composition of any of claims 147-161, wherein the mRNA is at a concentration of 0.4 to 0.8 mg/ml.

163. The composition of claim 162, wherein the mRNA is at a concentration of about 0.6 mg/mi.

164. The composition of any of claims 147-163, comprising:
a. an mRNA at a concentration of about 0.6 mg/ml encapsulated in the lipid nanoparticle,
b. trehalose at a concentration of about 8% w/v, and
c. TPGS at a concentration of about 0.5% w/v.

165. The composition of any of claims 147-163, comprising:
a. an mRNA at a concentration of about 0.6 mg/ml encapsulated in the lipid nanoparticle, and
b. sucrose at a concentration of about 8% w/v.

166. The composition of any of claims 147-163, comprising:
a. an mRNA encapsulated in the lipid nanoparticle,
b. a disaccharide such as trehalose or sucrose at a concentration of about 3-10% w/v,
c. a buffer, optionally a phosphate buffer, and
d. a salt, optionally sodium chloride.

167. The composition of claim 166, wherein:
a. the mRNA is at a concentration of 0.4 to 0.8 mg/ml,
b. the trehalose or sucrose is at a concentration of about 4% to 6% w/v,
c. the buffer is a phosphate buffer at a concentration of 1 mM to 10 mM (pH 5-5.5), and
d. the salt is sodium chloride at a concentration of at least 75 mM.

168. The composition of claim 167, wherein the sodium chloride is at a concentration of about 75 mm to about 200 mM.

169. The composition of any of claims 166-168, wherein:
a. the mRNA is at a concentration of about 0.4 mg/ml,
b. the disaccharide is sucrose at a concentration of about 4% w/v,
c. the buffer is a phosphate buffer at a concentration of about 2.5 mM (pH 5.5), and
d. the salt is sodium chloride at a concentration of about 150 mM.

170. The composition of any of claims 166-168, wherein:
a. the mRNA is at a concentration of about 0.4 mg/ml,
b. the disaccharide is trehalose at a concentration of about 4% w/v,
c. the buffer is a phosphate buffer at a concentration of about 10 mM (pH 5), and
d. the salt is sodium chloride at a concentration of about 150 mM.

171. The lipid nanoparticle or composition of any preceding claim for use in therapy, wherein the mRNA encodes a therapeutic protein and the therapy comprises administering the lipid nanoparticle or composition by nebulization.

172. The lipid nanoparticle or composition for use according to claim 171, wherein the lipid nanoparticle or composition is administered with a nebulizer comprising vibrating mesh technology (VMT).

173. The lipid nanoparticle or composition for use according to claim 171 or claim 172, wherein the lipid nanoparticle or composition is provided:
(i) in lyophilized form and reconstituted into an aqueous solution prior to nebulization; or
(ii) as a dry powder formulation.

174. The lipid nanoparticle or composition for use according to any one of claims 171-173, wherein the mRNA is delivered to the lungs.

175. The lipid nanoparticle or composition for use according to claim 174, wherein the therapeutic protein encoded by the mRNA is expressed in the lung.

176. The lipid nanoparticle or composition for use according to any one of claims 171-175, wherein the therapeutic protein is a secreted protein.

177. The lipid nanoparticle or composition for use according to any one of claims 171-175, wherein the therapeutic protein is an antibody.

178. The lipid nanoparticle or composition for use according to any one of claims 171-177, wherein the therapy comprises treating or preventing a disease or disorder in a subject.

179. The lipid nanoparticle or composition for use according to claim 178, wherein the disease or disorder is selected from:
(i) a pulmonary disease or disorder, e.g., a chronic respiratory disease,
(ii) a protein deficiency, e.g., a protein deficiency affecting the lungs
(iii) a neoplastic disease, e.g., a tumor, and
(iv) an infectious disease.

180. The lipid nanoparticle or composition for use according to claim 178 or claim 179, wherein the disease or disorder is a protein deficiency.

181. The lipid nanoparticle or composition for use according to claim 178, wherein the mRNA encodes the deficient protein.

182. The lipid nanoparticle or composition for use according to claim 180 or claim 181, wherein the protein deficiency is cystic fibrosis.

183. The lipid nanoparticle or composition for use according to claim 182, wherein the mRNA encodes CFTR.

184. The lipid nanoparticle or composition for use according to claim 180 or claim 181, wherein the protein deficiency is primary ciliary dyskinesia.

185. The lipid nanoparticle or composition for use according to claim 180 or claim 181, wherein the protein deficiency is a surfactant deficiency.

186. The lipid nanoparticle or composition for use according to claim 185, wherein the mRNA encodes a surfactant protein.

187. The lipid nanoparticle or composition for use according to claim 179, wherein the disease or disorder is a chronic respiratory disease.

188. The lipid nanoparticle or composition for use according to claim 187, wherein the chronic respiratory disease is chronic obstructive pulmonary disease (COPD), asthma, pulmonary arterial hypertension or idiopathic pulmonary fibrosis.

189. The lipid nanoparticle or composition for use according to any one of claims 171-188, wherein the mRNA encodes a therapeutic protein for treating a symptom of a pulmonary disease or disorder.

190. The lipid nanoparticle or composition for use according to claim 189, wherein the mRNA encodes an antibody directed against a pro-inflammatory cytokine.

191. The lipid nanoparticle or composition for use according to claim 179, wherein the disease or disorder is a neoplastic disease, e.g., a tumor.

192. The lipid nanoparticle or composition for use according to claim 191, wherein the mRNA encodes an antibody targeting a protein expressed on the surface of neoplastic cells, e.g., the cells making up the tumor.

193. The lipid nanoparticle or composition for use according to claim 179, wherein the disease or disorder is an infectious disease.

194. The lipid nanoparticle or composition for use according to claim 193, wherein the mRNA encodes an antigen derived from a causative agent of the infections disease.

195. The lipid nanoparticle or composition for use according to claim 193, wherein the infectious disease is caused by a virus.

196. The lipid nanoparticle or composition for use according to claim 195, wherein the mRNA encodes
(i) a soluble decoy receptor that binds a surface protein of the virus; or
(ii) an antibody directed to a surface protein of the virus.

197. The lipid nanoparticle or composition for use according to claim 193, wherein the infectious disease is caused by a bacterium.

198. The lipid nanoparticle or composition for use according to claim 197, wherein the mRNA encodes an antibody directed to a surface protein of the bacterium.

199. The lipid nanoparticle or composition for use according to any one of claims 171-198, wherein the subject is human.

200. A method for delivering mRNA which encodes a therapeutic protein in vivo comprising administering the lipid nanoparticle according to any one of claims 1-146 or the composition according to any one of claims 147-170 via pulmonary delivery to a subject, wherein the pulmonary delivery is via inhalation, and the composition is nebulized prior to inhalation.

201. The method according to claim 200, wherein the composition is provided in lyophilized form and reconstituted in an aqueous solution prior to nebulization.

202. The method according to claim 200 or claim 201, wherein the mRNA is delivered to the lungs.

203. The method according to claim 202, wherein the therapeutic protein encoded by the mRNA is expressed in the lung.

204. The method according to any one of claims 200-203, wherein the therapeutic protein is a secreted protein.

205. The method according to any one of claims 200-204, wherein the therapeutic protein is an antibody or an antigen.

206. A method of treating or preventing a disease or disorder in a subject, the method comprising administering the lipid nanoparticle according to any one of claims 1-146 or the composition according to any one of claims 147-170 via nebulization.

207. The method according to claim 206, wherein the disease or disorder is selected from:
(i) a pulmonary disease or disorder, e.g., a chronic respiratory disease,
(ii) a protein deficiency, e.g., a protein deficiency affecting the lung,
(iii) a neoplastic disease, e.g., a tumor, and
(iv) an infectious disease.

208. The method according to claim 207, wherein the pulmonary disease or disorder is a protein deficiency.

209. The method of claim 208, wherein the mRNA encodes the deficient protein.

210. The method according to claim 208 or claim 209, wherein the protein deficiency is cystic fibrosis.

211. The method of claim 210, wherein the mRNA encodes CFTR.

212. The method according to of claim 208 or claim 209, wherein the protein deficiency is primary ciliary dyskinesia.

213. The method of claim 208 or claim 209, wherein the protein deficiency is a surfactant deficiency.

214. The method of claim 213, wherein the mRNA encodes a surfactant protein.

215. The method according to claim 207, wherein the pulmonary disease or disorder is a chronic respiratory disease.

216. The method of claim 215, wherein the chronic respiratory disease is chronic obstructive pulmonary disease (COPD), asthma, pulmonary arterial hypertension or idiopathic pulmonary fibrosis.

217. The method according to any one of claims 207-216, wherein the mRNA encodes a therapeutic protein for treating a symptom of a pulmonary disease or disorder.

218. The method of claim 217, wherein the mRNA encodes an antibody directed against a pro-inflammatory cytokine.

219. The method of claim 207, wherein the disease or disorder is a neoplastic disease, e.g., a tumor.

220. The method of claim 219, wherein the mRNA encodes an antibody targeting a protein expressed on the surface of neoplastic cells, e.g., the cells making up the tumor.

221. The method of claim 207, wherein the disease or disorder is an infectious disease.

222. The method of claim 221, wherein the infectious disease is caused by a virus.

223. The method of claim 222, wherein the mRNA encodes a soluble decoy receptor that binds a surface protein of the virus.

224. The method of claim 222, wherein the mRNA encodes an antibody directed to a surface protein of the virus.

225. The method of claim 221, wherein the infectious disease is caused by a bacterium.

226. The method of claim 225, wherein the mRNA encodes an antibody directed to a surface protein of the bacterium.

227. The method of claim 221, wherein the mRNA encodes an antigen derived from a causative agent of the infections disease.

228. The method according to any one of claims 206-227 wherein the subject is human.
